SEQUENCE LISTING (78 sequences)

SEQ ID NO: 1 - protein, 449 aa, Artificial Sequence
  Description of Artificial Sequence Synthetic polypeptide
EVQLLESGGGLVQPGGSLRLSCAASGFTFSSYAMSWVRQAPGKGLEWVSEISGGGGYTDYADSVKGRFTISRDNSKNTLYLQMNSLRAEDTAVYYCARQMYYYYGMDVWGQVTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Artificial Sequence
  Description of Artificial Sequence Synthetic polypeptide
EIVLTQSPGTLSLSPGERATLSCRASQSVSANYLAWYQQKPGQAPRLLIYGASSRATGIPDRFSGSGSGTDFTLTISRLEPEDFAVYYCQQSYQRPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 243 aa, Artificial Sequence
  Description of Artificial Sequence Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 4 - protein, 712 aa, Artificial Sequence
  Description of Artificial Sequence Synthetic polypeptide
EVQLLESGGGLVQPGGSLRLSCAASGFTFSSYAMSWVRQAPGKGLEWVSEISGGGGYTDYADSVKGRFTISRDNSKNTLYLQMNSLRAEDTAVYYCARQMYYYYGMDVWGQVTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 215 aa, Artificial Sequence; identical to SEQ ID NO: 2
  Description of Artificial Sequence Synthetic polypeptide

SEQ ID NO: 6 - protein, 707 aa, Artificial Sequence
  Description of Artificial Sequence Synthetic polypeptide
EVQLLESGGGLVQPGGSLRLSCAASGFTFSSYAMSWVRQAPGKGLEWVSEISGGGGYTDYADSVKGRFTISRDNSKNTLYLQMNSLRAEDTAVYYCARQMYYYYGMDVWGQVTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 7 - protein, 215 aa, Artificial Sequence; identical to SEQ ID NO: 2
  Description of Artificial Sequence Synthetic polypeptide

SEQ ID NO: 8 - protein, 485 aa, Artificial Sequence
  Description of Artificial Sequence Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 449 aa, Artificial Sequence
  Description of Artificial Sequence Synthetic polypeptide
EVQLLESGGGLVQPGGSLRLSCAASGFTFSSYAMSWVRQAPGKGLEWVSEISGGGGYTDYADSVKGRFTISRDNSKNTLYLQMNSLRAEDTAVYYCARQMYYYYGMDVWGQVTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPCRDELTKNQVSLWCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 215 aa, Artificial Sequence; identical to SEQ ID NO: 2
  Description of Artificial Sequence Synthetic polypeptide

SEQ ID NO: 11 - protein, 712 aa, Artificial Sequence
  Description of Artificial Sequence Synthetic polypeptide
EVQLLESGGGLVQPGGSLRLSCAASGFTFSSYAMSWVRQAPGKGLEWVSEISGGGGYTDYADSVKGRFTISRDNSKNTLYLQMNSLRAEDTAVYYCARQMYYYYGMDVWGQVTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 449 aa, Artificial Sequence; identical to SEQ ID NO: 9
  Description of Artificial Sequence Synthetic polypeptide

SEQ ID NO: 13 - protein, 215 aa, Artificial Sequence; identical to SEQ ID NO: 2
  Description of Artificial Sequence Synthetic polypeptide

SEQ ID NO: 14 - protein, 707 aa, Artificial Sequence
  Description of Artificial Sequence Synthetic polypeptide
EVQLLESGGGLVQPGGSLRLSCAASGFTFSSYAMSWVRQAPGKGLEWVSEISGGGGYTDYADSVKGRFTISRDNSKNTLYLQMNSLRAEDTAVYYCARQMYYYYGMDVWGQVTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 15 - protein, 449 aa, Artificial Sequence; identical to SEQ ID NO: 9
  Description of Artificial Sequence Synthetic polypeptide

SEQ ID NO: 16 - protein, 215 aa, Artificial Sequence; identical to SEQ ID NO: 2
  Description of Artificial Sequence Synthetic polypeptide

SEQ ID NO: 17 - protein, 119 aa, Artificial Sequence
  Description of Artificial Sequence Synthetic polypeptide
EVQLLESGGGLVQPGGSLRLSCAASGFTFSSYAMSWVRQAPGKGLEWVSEISGGGGYTDYADSVKGRFTISRDNSKNTLYLQMNSLRAEDTAVYYCARQMYYYYGMDVWGQVTTVTVSS

SEQ ID NO: 18 - protein, 108 aa, Artificial Sequence
  Description of Artificial Sequence Synthetic polypeptide
EIVLTQSPGTLSLSPGERATLSCRASQSVSANYLAWYQQKPGQAPRLLIYGASSRATGIPDRFSGSGSGTDFTLTISRLEPEDFAVYYCQQSYQRPLTFGQGTKVEIK

SEQ ID NO: 19 - protein, 10 aa, Artificial Sequence
  Description of Artificial Sequence Synthetic peptide
  MOD_RES (4)..(4): Phosphorylated serine
RVASPTSGVK

SEQ ID NO: 20 - protein, 8 aa, Artificial Sequence
  Description of Artificial Sequence Synthetic peptide
GFTFSSYA

SEQ ID NO: 21 - protein, 8 aa, Artificial Sequence
  Description of Artificial Sequence Synthetic peptide
ISGGGGYT

SEQ ID NO: 22 - protein, 12 aa, Artificial Sequence
  Description of Artificial Sequence Synthetic peptide
ARQMYYYYGMDV

SEQ ID NO: 23 - protein, 7 aa, Artificial Sequence
  Description of Artificial Sequence Synthetic peptide
QSVSANY

SEQ ID NO: 24 - protein, 3 aa, Artificial Sequence
  Description of Artificial Sequence Synthetic peptide
GAS

SEQ ID NO: 25 - protein, 9 aa, Artificial Sequence
  Description of Artificial Sequence Synthetic peptide
QQSYQRPLT

SEQ ID NO: 26 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 27 - protein, 330 aa, Homo sapiens [sequence omitted]

SEQ ID NO: 28 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 29 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 30 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 31 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 33 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 34 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 36 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 38 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 39 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 40 - protein, 8 aa, Artificial Sequence
  Description of Artificial Sequence Synthetic peptide
  MOD_RES (4)..(4): Phosphorylated serine
KMDSLDMQ

SEQ ID NO: 41 - protein, 11 aa, Artificial Sequence
  Description of Artificial Sequence Synthetic peptide
  MOD_RES (7)..(7): Phosphorylated serine
KLIDIVSSQKV

SEQ ID NO: 42 - protein, 10 aa, Artificial Sequence
  Description of Artificial Sequence Synthetic peptide
  MOD_RES (4)..(4): Phosphorylated serine
YLDSGIHSGA

SEQ ID NO: 43 - protein, 18 aa, Artificial Sequence
  Description of Artificial Sequence Synthetic peptide
GEGTSTGSGGSGGSGGAD

SEQ ID NO: 44 - protein, 15 aa, Artificial Sequence
  Description of Artificial Sequence Synthetic peptide
GGGGSGGGGSGGGGS

SEQ ID NO: 45 - protein, 10 aa, Artificial Sequence
  Description of Artificial Sequence Synthetic peptide
GGGGSGGGGS

SEQ ID NO: 46 - protein, 6 aa, Artificial Sequence
  Description of Artificial Sequence Synthetic 6xHis tag
HHHHHH

SEQ ID NO: 47 - protein, 9 aa, Artificial Sequence
  Description of Artificial Sequence Synthetic peptide
  MOD_RES (4)..(4): Phosphorylated serine
RVASPTSGV

SEQ ID NO: 48 - protein, 11 aa, Artificial Sequence
  Description of Artificial Sequence Synthetic peptide
ARVASPTSGVK

SEQ ID NO: 49 - protein, 12 aa, Artificial Sequence
  Description of Artificial Sequence Synthetic peptide
ARVASPTSGVKR

SEQ ID NO: 50 - protein, 9 aa, Homo sapiens
RVASPTSGV

SEQ ID NO: 51 - protein, 10 aa, Homo sapiens
RVASPTSGVK

SEQ ID NO: 52 - protein, 9 aa, Artificial Sequence
  Description of Artificial Sequence Synthetic peptide
  MOD_RES (5)..(5): Phosphorylated serine
GLLGSPVRA

SEQ ID NO: 53 - protein, 9 aa, Homo sapiens
RMFPNAPYL

SEQ ID NO: 54 - protein, 9 aa, Alphapapillomavirus sp.
YMLDLQPET

SEQ ID NO: 55 - protein, 9 aa, Homo sapiens
QLQNPSYDK

SEQ ID NO: 56 - protein, 9 aa, Artificial Sequence
  Description of Artificial Sequence Synthetic peptide
  MOD_RES (4)..(4): Phosphorylated serine
AVASPTSGV

SEQ ID NO: 57 - protein, 9 aa, Artificial Sequence
  Description of Artificial Sequence Synthetic peptide
  MOD_RES (4)..(4): Phosphorylated serine
RAASPTSGV

SEQ ID NO: 58 - protein, 9 aa, Artificial Sequence
  Description of Artificial Sequence Synthetic peptide
  MOD_RES (4)..(4): Phosphorylated serine
RVGSPTSGV

SEQ ID NO: 59 - protein, 9 aa, Artificial Sequence
  Description of Artificial Sequence Synthetic peptide
RVAAPTSGV

SEQ ID NO: 60 - protein, 9 aa, Artificial Sequence
  Description of Artificial Sequence Synthetic peptide
  MOD_RES (4)..(4): Phosphorylated serine
RVASATSGV

SEQ ID NO: 61 - protein, 9 aa, Artificial Sequence
  Description of Artificial Sequence Synthetic peptide
  MOD_RES (4)..(4): Phosphorylated serine
RVASPASGV

SEQ ID NO: 62 - protein, 9 aa, Artificial Sequence
  Description of Artificial Sequence Synthetic peptide
  MOD_RES (4)..(4): Phosphorylated serine
RVASPTAGV

SEQ ID NO: 63 - protein, 9 aa, Artificial Sequence
  Description of Artificial Sequence Synthetic peptide
  MOD_RES (4)..(4): Phosphorylated serine
RVASPTSAV

SEQ ID NO: 64 - protein, 9 aa, Artificial Sequence
  Description of Artificial Sequence Synthetic peptide
  MOD_RES (4)..(4): Phosphorylated serine
RVASPTSGA

SEQ ID NO: 65 - protein, 9 aa, Artificial Sequence
  Description of Artificial Sequence Synthetic peptide
  MOD_RES (4)..(4): Phosphorylated threonine
RVATPTSGV

SEQ ID NO: 66 - protein, 9 aa, Homo sapiens
RVATPTSGV

SEQ ID NO: 67 - protein, 9 aa, Artificial Sequence
  Description of Artificial Sequence Synthetic peptide
  MOD_RES (4)..(4): Phosphorylated serine
RLDSYVRSL

SEQ ID NO: 68 - protein, 9 aa, Artificial Sequence
  Description of Artificial Sequence Synthetic peptide
  MOD_RES (4)..(4): Phosphorylated serine
KMDSFLDMQ

SEQ ID NO: 69 - protein, 10 aa, Artificial Sequence
  Description of Artificial Sequence Synthetic peptide
  MOD_RES (4)..(4): Phosphorylated serine
RTYSGPMNKV

SEQ ID NO: 70 - protein, 10 aa, Artificial Sequence
  Description of Artificial Sequence Synthetic peptide
  MOD_RES (4)..(4): Phosphorylated serine
RQASIELPSM

SEQ ID NO: 71 - protein, 9 aa, Artificial Sequence
  Description of Artificial Sequence Synthetic peptide
  MOD_RES (4)..(4): Phosphorylated serine
RQASLSISV

SEQ ID NO: 72 - protein, 9 aa, Artificial Sequence
  Description of Artificial Sequence Synthetic peptide
  MOD_RES (4)..(4): Phosphorylated serine
RTFSPTYGL

SEQ ID NO: 73 - protein, 10 aa, Artificial Sequence
  Description of Artificial Sequence Synthetic peptide
  MOD_RES (4)..(4): Phosphorylated serine
RQLSSGVSEI

SEQ ID NO: 74 - protein, 9 aa, Artificial Sequence
  Description of Artificial Sequence Synthetic peptide
  MOD_RES (4)..(4): Phosphorylated serine
RLQSTSERL

SEQ ID NO: 75 - protein, 9 aa, Artificial Sequence
  Description of Artificial Sequence Synthetic peptide
  MOD_RES (4)..(4): Phosphorylated serine
RQISQDVKL

SEQ ID NO: 76 - DNA, 22 bp, Artificial Sequence
  Description of Artificial Sequence Synthetic oligonucleotide
tcttgcacttgtcacgaattcg

SEQ ID NO: 77 - DNA, 40 bp, Artificial Sequence
  Description of Artificial Sequence Synthetic oligonucleotide
taatagggttcgatccctaccggttagtaatgagtttgat

SEQ ID NO: 78 - protein, 8 aa, Artificial Sequence
  Description of Artificial Sequence Synthetic peptide
  MOD_RES (3)..(3): Phosphorylated serine
VASPTSGV

(19) United States
(12) Patent Application Publication
(10) Pub. No.: US 2024/0239899 A1
(43) Pub. Date: Jul. 18, 2024

(54) TCR MIMIC MONOCLONAL ANTIBODIES REACTIVE WITH THE PHOSPHO-NEOANTIGEN PIRS2/HLA-A*02:01 COMPLEX AND USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US); Tri-Institutional Therapeutics Discovery Institute, Inc., New York, NY (US)

(72) Inventors: Tao DAO, New York, NY (US); David A. SCHEINBERG, New York, NY (US); Paul BALDERES, New York, NY (US); Mary Ann POHL, New York, NY (US)

(21) Appl. No.: 18/561,699

(22) PCT Filed: May 19, 2022
(86) PCT No.: PCT/US22/29974

§ 371 Date: Nov. 16, 2023

Related U.S. Application Data
(60) Provisional application No. 63/191,093, filed on May 20, 2021.
(60) Provisional application No. 63/308,650, filed on Feb. 10, 2022.

(51) Int. Cl. C07K 16/28; A61K 38/21; A61K 39/00; A61P 35/02; C07K 14/705

(52) U.S. Cl. CPC C07K 16/2833; A61K 38/217; A61P 35/02; C07K 14/70503; A61K 2039/505; C07K 2317/31; C07K 2317/32; C07K 2317/622; C07K 2317/732

(57) ABSTRACT

The present technology relates generally to compositions that specifically recognize and bind to a serine-phosphorylated IRS2 (pIRS2) peptide RVA[pS]PTSGVK (SEQ ID NO: 19) complexed with a major histocompatibility antigen (e.g., HLA-A*02). The compositions of the present technology are useful in methods for treating pIRS2-associated diseases (e.g., cancers) in a subject in need thereof.

### CROSS-REFERENCE TO RELATED APPLICATIONS

This application claims the benefit of and priority to U.S. Provisional Patent Application No. 63/191,093, filed May 20, 2021, and U.S. Provisional Patent Application No. 63/308,650, filed Feb. 10, 2022, the entire contents of which are incorporated herein by reference.

### STATEMENT OF GOVERNMENT INTEREST

This invention was made with government support under CA55349, CA23766 and CA241894-01A1, awarded by the National Institutes of Health. The government has certain rights in the invention.

### TECHNICAL FIELD

The present technology relates generally to compositions that specifically bind to a serine-phosphorylated IRS2 (pIRS2) peptide RVA[pS]PTSGVK (SEQ ID NO: 19) complexed with MHC, including antibodies such as human, humanized, or chimeric antibodies, antibody fragments, chimeric antibody-T cell receptors (caTCRs), chimeric antigen receptors (CARs), fusion proteins, and conjugates thereof. The compositions of the present technology bind to HLA-A*02-restricted pIRS2 peptides and are useful for the treatment of pIRS2-associated diseases, including but not limited to cancers.

### BACKGROUND

The following description of the background of the present technology is provided simply as an aid in understanding the present technology and is not admitted to describe or constitute prior art to the present technology.

The long-term clinical responses in patients with a variety of cancers after checkpoint blockade therapy has demonstrated that T cells directed against cancer neoantigens (neoAgs) arising from tumor-specific gene mutations play a crucial role in the anti-cancer immunity (Schumacher T, Schreiber R D (2015) Science 348:69-74; Luksza M el al (2017) Nature 551:517-520; McGranahan N et a/(2016). Science 351(6280):1463-1469). These neoAgs are potentially immunogenic because they are not expressed in normal tissues and therefore not subject to central T cell tolerance. Although neoAgs have long been envisioned as ideal targets for immunotherapy, a small number of such neoAgs has proven to be clinically useful in unique individuals by use of adoptive T cell therapy including melanoma and epithelial cancers and several patient-specific vaccines are being tested (Tran E et al (2015) Science 350:1387-1390; Carreno, B M et a/(2015). Science 348:803-808). However, predicted neoepitopes that trigger bona fide antitumor immune responses in patients are still rare and difficult and expensive to identify (Finn O J, Rammensee H G (2018). Cold Spring Harb Perspect Biol 10. pii: a028829; The problem with neoantigen prediction. Nat Biotech. 35, 97 (2017)). Importantly, the mutations are nonsynonymous and patient-specific (“private”) and can be used in only a single patient typically. These features of neoAgs make them less suitable for wide clinical translation.

Accordingly, there is a need for therapeutics agents that target that are tumor specific “public” neoAgs that are widely expressed and not patient-specific.

### SUMMARY OF THE PRESENT TECHNOLOGY

The present disclosure identifies and characterizes immunoglobulin-related compositions (e.g., anti bodies including human, humanized, or chimeric antibodies, antibody fragments, chimeric antibody-T cell receptors (caTCRs), chimeric antigen receptors (CARs), fusion proteins, and conjugates thereof) that are able to target cytosolic/intracellular proteins, for example, pIRS2. The disclosed immunoglobulin-related compositions target a peptide/MHC complex as it would typically appear on the surface of a cell following antigen processing of pIRS2 protein and presentation by the cell. In that regard, the immunoglobulin-related compositions mimic T-cell receptors in that the immunoglobulin-related compositions have the ability to specifically recognize and bind to a peptide in an MHC-restricted fashion, that is, when the peptide is bound to an MHC antigen. The peptide/MHC complex recapitulates the antigen as it would typically appear on the surface of a cell following antigen processing of the pIRS2 protein, which in turn is presented to a T-cell. The immunoglobulin-related compositions disclosed herein specifically recognize and bind to epitopes of a peptide/HLA-A*02 complex, particularly a pIRS2/HLA-A*02 complex. Examples of peptides that are recognized by the immunoglobulin-related compositions of the present disclosure as part of an HLA-peptide complex include, for example, a peptide with the amino acid sequence RVA[pS]PTSGVK (SEQ ID NO: 19).

In one aspect, the present disclosure provides compositions such as antigen binding proteins or immunoglobulin-related compositions comprising antibody moieties that specifically bind to pIRS2 peptide/MHC complexes (also called “anti-pIRS2 peptide/MHC” or “anti-pIRS2/MHC” herein). Such compositions can comprise, consist essentially of, or consist of, e.g., anti-pIRS2 peptide/MHC antibodies or antigen binding fragments thereof, chimeric antigen receptors (CARs), fusion proteins, and conjugates. The antibody moieties can comprise: a heavy chain immunoglobulin variable domain (V_{H}) comprising a V_{H}-CDR1 sequence, a V_{H}-CDR2 sequence, and a V_{H}-CDR3 sequence of the V_{H} sequence of SEQ ID NO: 17, and a light chain immunoglobulin variable domain (V_{L}) comprising a V_{L}-CDR1 sequence, a V_{L}-CDR2 sequence, and a V_{L}-CDR3 sequence of the V_{L} sequence SEQ ID NO: 18. The antibody moiety may be a full-length antibody, a Fab, a F(ab′)₂, a Fab′, a Fᵥ, or a single chain Fv (scFv).

In some embodiments of the antigen binding proteins or immunoglobulin-related compositions disclosed herein, the V_{H}-CDR1 sequence comprises the sequence of SEQ ID NO: 20, the V_{H}-CDR2 sequence comprises the sequence of SEQ ID NO: 21, the V_{H}-CDR3 sequence comprises the sequence of SEQ ID NO: 22, the V_{L}-CDR1 sequence comprises the sequence of SEQ ID NO: 23, the V_{L}-CDR2 sequence comprises the sequence of SEQ ID NO: 24, and the V_{L}-CDR3 sequence comprises the sequence of SEQ ID NO: 25.

Additionally or alternatively, in some embodiments of the antigen binding proteins or immunoglobulin-related compositions disclosed herein, (a) the V_{H} comprises an amino acid sequence having at least 90% identity to SEQ ID NO: 17, and/or (b) the V_{L} comprises an amino acid sequence having at least 90% identity to SEQ ID NO: 18. In certain embodiments, (a) the V_{H} comprises an amino acid sequence of SEQ ID NO: 17 or a variant thereof having one or more conservative amino acid substitutions; and/or (b) the V_{L} comprises an amino acid sequence of SEQ ID NO: 18 or a variant thereof having one or more conservative amino acid substitutions.

Additionally or alternatively, in some embodiments, the antigen binding proteins or immunoglobulin-related compositions disclosed herein comprise an amino acid sequence having at least 90% identity to a sequence selected from the group consisting of: SEQ ID NOs: 1-2, or 4-16. In certain embodiments, the antigen binding proteins or immunoglobulin-related compositions of the present technology comprise an amino acid sequence selected from the group consisting of: SEQ ID NOs: 1-2, or 4-16.

In any of the preceding embodiments, the antigen binding proteins or immunoglobulin-related compositions of the present technology further comprise a Fc domain of an isotype selected from the group consisting of IgG1, IgG2, IgG3, IgG4, IgA1, IgA2, IgM, IgD, and IgE.

Additionally or alternatively, in some embodiments, the antigen binding proteins or immunoglobulin-related compositions of the present technology are chimeric antibody-T cell receptors (caTCR) and/or comprise at least a fragment of a T cell receptor (TCR) chain. In some embodiments, the fragment of TCR chain comprises the transmembrane domain of the TCR chain. In certain embodiments, the fragment of TCR chain does not comprise any CDR sequence of the TCR chain. Additionally or alternatively, in some embodiments, the antigen binding proteins or immunoglobulin-related compositions of the present technology are chimeric antigen receptors (CAR).

In any and all of the preceding embodiments, the antigen binding proteins or immunoglobulin-related compositions of the present technology may be monospecific, multispecific, or bispecific. In some embodiments, the antigen binding proteins or immunoglobulin-related compositions of the present technology comprise a tandem scFv, a diabody (Db), a single chain diabody scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody. Additionally or alternatively, in some embodiments, the antigen binding proteins or immunoglobulin-related compositions of the present technology comprise a tandem scFv with at least one peptide linker between two scFvs. In certain embodiments, the antigen binding proteins or immunoglobulin-related compositions of the present technology comprise a second antibody moiety that specifically binds to a second antigen. The second antigen may be a disease-specific antigen that is not pIRS2/MHC, or an antigen on the surface of a T cell, a natural killer cell, a neutrophil, a. monocyte, a macrophage, or a dendritic cell.

In any and all of the preceding embodiments, the antigen binding proteins or immunoglobulin-related compositions of the present technology may be a monoclonal antibody, a chimeric antibody, a humanized antibody, or a human antibody. In some embodiments, the antigen binding protein or immunoglobulin-related composition of the present technology is a fully human antibody. Additionally or alternatively, in some embodiments, the antigen binding proteins or immunoglobulin-related compositions of the present technology may be an immunoglobulin polypeptide, or an immunoglobulin-like polypeptide.

Additionally or alternatively, in some embodiments, the antigen binding proteins or immunoglobulin-related compositions of the present technology specifically bind to a pIRS2 peptide complexed with HLA-A*02. The HLA-A*02 may be HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50. In certain embodiments, the pIRS2 peptide comprises the amino acid sequence RVA[pS]PTSGVK (SEQ ID NO: 19).

In one aspect, the present disclosure provides an anti-pIRS2/MHC composition comprising an antibody moiety that competes with the any of the antigen binding proteins or immunoglobulin-related compositions of the present technology for specific binding to a pIRS2/MHC complex.

In one aspect, the present disclosure provides recombinant nucleic acids or a set of recombinant nucleic acids encoding any and all embodiments of the antigen binding proteins or immunoglobulin-related compositions described herein, with all components of the composition encoded by one nucleic acid or by the set of nucleic acids. In another aspect, the present disclosure provides a vector comprising said recombinant nucleic acids, as well as a set of vectors comprising said set of recombinant nucleic acids. Also disclosed herein are cells comprising any of the recombinant nucleic acids, set of recombinant nucleic acids, vectors, or set of vectors disclosed herein, as well as cells that display on its surface or secrete any of the antigen binding proteins or immunoglobulin-related compositions of the present technology. The cells may be a T cell, a NK cell, a B cell, or a monocyte/macrophage.

In one aspect, the present disclosure provides a pharmaceutical compositions comprising the antigen binding proteins or immunoglobulin-related compositions of the present technology, as well as any of the recombinant nucleic acids, set of recombinant nucleic acids, vectors, set of vectors, or cells disclosed herein, together with a pharmaceutically acceptable carrier.

Additionally or alternatively, in some embodiments, the antigen binding proteins or immunoglobulin-related compositions of the present technology are conjugated to an agent selected from the group consisting of detectable label, isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

In one aspect, the present disclosure provides a method for detecting pIRS2 expression levels in a biological sample comprising (a) contacting the biological sample with any of the antigen binding proteins or immunoglobulin-related compositions of the present technology; and (b) detecting binding to a pIRS2 peptide-HLA-A*02 complex in the biological sample. In some embodiments, the pIRS2 peptide comprises the amino acid sequence RVA[pS]PTSGVK (SEQ ID NO: 19).

In another aspect, the present disclosure provides methods for treating a pIRS2-associated disease in a subject in need thereof, comprising administering to the subject an effective amount of any of the antigen binding proteins or immunoglobulin-related compositions disclosed herein, or any of the recombinant nucleic acids, set of recombinant nucleic acids, vectors, set of vectors, or cells disclosed herein, or any of the pharmaceutical compositions disclosed herein. In certain embodiments, the methods disclosed herein further comprise administering to the subject an effective amount of interferon-γ.

Additionally or alternatively, in some embodiments of the methods disclosed herein, the pIRS2-associated disease is a cancer. Examples of cancer include, but are not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, ovarian cancer, breast cancer, cervical cancer, prostate cancer, melanoma, mesothelioma, pancreatic cancer, thyroid cancer, liver cancer, hepatocellular carcinoma, or a cancer presenting the peptide of RVA[pS]PTSGVK (SEQ ID NO: 19) in complex with HLA-A*02.

Additionally or alternatively, in some embodiments, the methods of the present technology further comprise separately, sequentially or simultaneously administering at least one additional therapeutic agent to the subject. Examples of additional therapeutic agents include, but are not limited to alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, immune checkpoint inhibitors, monoclonal antibodies that specifically target tumor antigens, T-cell therapy, immune activating agents, oncolytic virus therapy and cancer vaccines.

Also disclosed herein are kits comprising any of the antigen binding proteins or immunoglobulin-related compositions of the present technology and instructions for use. In some embodiments of the kits of the present technology, the antigen binding proteins or immunoglobulin-related compositions are coupled to at least one detectable label selected from the group consisting of a radioactive label, a fluorescent label, and a chromogenic label. Additionally or alternatively, in some embodiments, the kits further comprise a secondary antibody that specifically binds to the antigen binding proteins or immunoglobulin-related compositions of the present technology.

### BRIEF DESCRIPTION OF THE DRAWINGS

FIGS. 1A-1D demonstrate binding of the 6B1 mAb of the present technology. FIG. 1A shows binding of 6B1 to T2 cells pulsed with or without peptides. pIRS2, WT-IRS2, pCDC25b or, WT1-RMF peptide at a concentration of 20 μg/ml was pulsed onto T2 cells overnight. Cells were washed and stained with 6B1 mAb at the concentration of 3 μg/ml, followed by 2ndary mAb staining. The staining included: Secondary mAb, or isotype control human IgG1. In parallel, HLA-A2 expression was determined by staining the cells with anti-HLA-A2 mAb BB7 clone (FIG. 1B). “Uns”=unstained. 6B1 titration was performed on T2 cells pulsed with indicated peptides and stained with indirect staining with 6B1 at concentrations ranging from 10 μg/ml to 0.1 μg/ml) (FIG. 1C). Binding kinetics of 6B1 was measured by Biolayer interferometry (BLI) as indicated in the Materials and methods (FIG. 1D).

FIGS. 2A-2J show epitope specificity and recognition of tumor cells of the 6B1 mAb of the present technology. The pIRS2 peptide sequence was substituted with alanine at positions 1, 2, 4, 5, 6, 7, 8, 9 or with glycine (G3) indicated as A1 to A9 and G3 and the binding of 6B1 (3 μg/ml) was determined by indirect staining and flow cytometric analysis. T2 cells alone, or pulsed with HPV peptide were the controls (FIG. 2A). The same cells were simultaneously stained with anti-HLA-A2 mAb, clone BB7.2, to measure the relative binding of the peptides to HLA-A2 molecule (FIG. 2B). Similarly, threonine substituted peptide with (p-T4) or without phosphate (WT-T4) at the position 4 was pulsed onto T2 cells and the binding of 6B1 mAb was determined by flow cytometry (FIG. 2C). The same cells were simultaneously stained with anti-HLA-A2 mAb, clone BB7.2, to measure the relative binding of the peptides to HLA-A2 molecule (FIG. 2D). Recognition of the naturally presented pIRS2/A2 complex on the tumor cell surface by 6B1 in a pIRS2/HLA-A2-restricted manner was determined by flow cytometric analysis. Human leukemia cell lines BV173 (FIG. 2E), SET2 (FIG. 2F), ovarian cancer cell line SKOV-3 (FIG. 2H) and Burkitt's lymphoma cell line Jeko (FIG. 2I), and HLA-A2 negative T leukemia cell line Jurkat (FIG. 2J) were stained with 6B1 conjugated to APC at 10 μg/ml followed by flow cytometric analysis. T2 cells pulsed with pIRS2 was used as a positive control (FIG. 2G). Unstained cells and isotype hIgG1 were used as negative controls. Data are representative of three experiments

FIGS. 3A-3C show that 6B1 mediates ADCC with human PBMC effectors at indicated concentrations. T2 cells alone, or pulsed with pIRS2 (FIG. 3A), WT-IRS2 (FIG. 3B) or irrelevant Ewing's sarcoma (EW) (FIG. 3C) peptides at 50 μg/ml, incubated with fresh human PBMC effectors at E:T ratio of 30:1 with cytotoxicity measured by 5 hr-⁵¹Cr-release assay. Each data point was the average of triplicate cultures ±SD and are representative of three similar experiments.

FIGS. 4A-4E show specificity of 6B1 bispecific antibodies (BisAbs) of the present technology. FIG. 4A shows a schematic of 6B1 BisAb panel. Five different BisAb formats engaging anti-CD3 mAb 2LK are shown as indicated. FIG. 4B-4E show binding of 6B1 BisAbs to T2 cells. T2 cells alone (FIG. 4B) or T2 cells pulsed with pIRS2 (FIG. 4D), WT-IRS2 (FIG. 4E) or, HPV (FIG. 4C) peptide at a concentration of 20 μg/ml, were stained with 6B1 BisAbs at the concentration of 1 or 0.1p g/ml, followed by secondary anti-His-tag mAb staining. The staining included: Secondary mAb, or isotype control human IgG1.

FIGS. 5A-5I demonstrate cytotoxicity by 6B1 BisAbs of the present technology. T2 cells alone (FIG. 5A), or pulsed with pIRS2 (FIG. 5B), WT-IRS2 (FIG. 5C) or irrelevant EW (FIG. 5D) peptides at 50 μg/ml, were incubated with PBMCs at an E:T of 30:1 and mAbs at indicated concentrations and the cytotoxicity was measured by 5 hr-⁵¹Cr-release assay. Each data point was the average of triplicate cultures ±SD and representative of two similar experiments. Similarly, T cell-mediated cytotoxicity against tumor cell lines MDA-MB-231 (FIG. 5E), SKOV-3 (FIG. 5F), TPC-1 (FIG. 5G) or A375 (FIG. 511) was measure by Lactate dehydrogenase (LDH)-release assay at an E:T ratio of 20:1, with BisAbs at the indicated concentrations. Each data point was the average of triplicate cultures ±SD and representative of three similar experiments. FIG. 5I: AML PDXs were stained with mAbs to CD33 and HLA-A2. The cells were labeled with CFSE and were incubated with activated T cells at an E:T ratio of 6:1, in the presence of 6B1 (1+1) or isotype control at 10 μg/ml. After overnight culture, cells were harvested, washed and stained with mAb to CD33. Percentage reduction of total CFSE positive cells was determined as killing of the cells. The percentage lysis of 6B1 (1+1) group was plotted over isotype control.

FIGS. 6A-6D show structural modeling of 6B1 in complex with phosphopeptide/HLA-A2 complexes. FIG. 6A shows linear regression of binding interface energy in Rosetta energy units (REU) with in vitro binding of 6B1 to the indicated phosphopeptide-complexes. Regression line passes through mean of top 10 scoring models for each phosphopeptide, shaded regions represent 95% CI, R-value represents Pearson correlation coefficient and respective two-tailed p-value. FIG. 6B shows representative model of 6B1 in complex with pIRS2/HLA-A2. 6B1 VH, VL, HLA-A2, and phosphopeptide shown in dark green, light green, mauve, and orange, respectively. Phosphoserine residue is colored red. FIG. 6C shows 6B1-pIRS2/HLA-A2 complex at different angles showing contacts of CDRH3 tyrosines (green) in contact with phosphopeptide (orange), with phosphoserine (SEP) distinguished in red. Phosphopeptide residues are labeled at their approximate locations. FIG. 6D shows 6B1-pKMD/HLA-A2 complex viewed at the same angles as in (FIG. 6C).

FIGS. 7A-7C show target validation. FIG. 7A shows that pIRS2 stimulated T cell responses do not have cross-reactivity to its nonphosphorylated peptide (WT-IRS2). Fresh CD3+ T cells from a healthy HLA-A*02:01+ donor were stimulated with pIRS2 peptide for 6 times. The peptide-specific response was measured by IFN-gamma ELISPOT assay. pIRS2 is the phosphorylated target peptide and IRS2 is the native cognate peptide. CD14 is normal negative control (autologous CD14+ antigen presenting cells) and EW is an irrelevant HLA-A2-binding peptide. Each point represents average +/−SD from triplicates cultures and three similar experiments. Phytohemagglutinin (PHA) activation with “too numerous to count” spots is used as an internal validation positive control in each experiment. Each point represents the average from triplicates cultures (p value ranges were <0.01 and representative of three similar experiments from three different donors. FIG. 7B shows Western blot assays used to examine expressions of both phosphorylated as well as the native IRS-2 protein in 20-30 μg of extracts from indicated cell lines. Blots were derived from replicate samples run on parallel gels. Each blot was probed with either anti-pSer1100-IRS2. FIG. 7C shows MS/MS data from immunoprecipitation with HLA-A, B, C specific antibody in five different cell lines detecting pIRS peptides in an A02 and A03 HLA-context.

FIG. 8 shows T cell-mediated cytotoxicity by 6B1 (1+1) against MDA-MB-231, SKOV-3, BV173, Jeko, NCEB1 and SET-2 cell lines was measured by LDH assay, using EBV-T cells at an E:T ratio of 10:1. Each data point was the average of triplicate cultures plus/minus SD and P value ranges were: BV173: 0.013 to 0.025; SKOV3/A2: 0.002 to 0.02; MDA-MB-231: 0.002 to 0.004; Jeko: 0.006 to 0.04; NCEB1: 0.008 to 0.03; SET-2: 0.015 to 0.034. The data are representative of three similar experiments from 3 different donors.

FIG. 9 shows cell lines used in the study. Protein was detected by western blot. Epitope was detected by MS. HLA-A*02:01 and 6B1 binding was detected by flow cytometry. Pos=positive. Neg=negative. NT=not tested.

FIG. 10 shows peptides used for initial characterization of mAbs specific for the pIRS2/HLA*-A02:01 complex.

FIG. 11 shows that 6B1 recognizes arginine at position 1 and phosphate at Ser-4 in a panel of phosphopeptides. Binding scores were indicated by strong (+++), Intermediate (++), weak (+). All peptides were pulsed onto T2 cells at 20 μg/ml and the binding of the mAb was determined by flow cytometric analysis. The sequences of the phosphopeptides were derived from Phopho Plus database.

FIG. 12 shows inter-residue contact energies between CDRH3 contacts and each phosphopeptide/HLA-A2 complex as determined by Rosetta3 scoring residue contacts of the lowest energy 6B1-phosphopeptide-HLA-A2 models for pIRS2 and pKMD.

FIG. 13 shows the amino acid sequences of the 6B1 light and heavy chains and L2K-scFv in different antibody formats (represented as SEQ ID NOs: 1-16). VH and VL domains of 6B1 and L2K are italicized, and linkers are underlined. Mutations to the Fc region are indicated in boldface.

FIGS. 14A-14F demonstrate that interferon-gamma (IFN-γ) enhances the binding of 6B1 mAb to tumor cells. To test if IFN-γ would enhance HLA-A2 expression and epitope processing, the tumor cell lines MDA-MB-231 (FIG. 14A), SET-2 (FIG. 14B), TPC-1 (FIG. 14C), BV173 (FIG. 14D), and Jurkat (FIG. 14E) were treated with human IFN-γ (100 ng/ml) for three days and the binding of the cells to 6B1 mAb and BB7 mAb (anti-HLA-A02) was measured by flow cytometric analysis. IFN-γ treatment significantly enhanced the binding of 6B1 to MDA-MB-231, SET-2, TPC-1 and in a lesser degree to BV173 cells, which was correlated to HLA-A2 upregulation. Jurkat cells are non-HA-A2 and no changes were seen after IFN-γ treatment. T2 cells pulsed with pIRS2 peptide (20 μg/ml) or irrelevant HLA-A2-binding peptide EW were used as positive and negative controls and experimental groups were indicated in red letters (FIG. 14F). Experimental groups in all other cell lines were indicated in upper and middle right, next to the SET-2 and BV173 cell lines.

FIGS. 15A-15G demonstrate epitope specificity of the 6B1 mAb of the present technology in non-cancerous HLA-A2 positive human control cells. FIGS. 15B-15D show normal human cardiomyocytes, cardiac fibroblasts and thymic fibroblasts and AML cell line AML-14 (FIG. 15A) were stained with 6B1 or isotype control (3 μg/ml) and followed by goat anti-human IgG Fab2 conjugated to FITC. HLA-A2 expression (FIG. 15E) was simultaneously measured by anti HLA-A2 mAb (clone BB7.2) conjugated to APC. Whole blood from HLA-A2 positive (FIG. 15F) or negative (FIG. 15G) healthy donor was stained with the 6B1 or isotype control (3 μg/ml), and mAbs to CD15, CD33 and CD45RA, lysed red blood cells, washed and ran on flow cytometry. The data represents staining from five separated experiments with multiple donors.

FIGS. 16A-16D demonstrate the effects of the 6B1 mAb of the present technology on IFN-gamma and TNF-alpha secretion in different cell lines.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, immunology, microbiology and recombinant DNA are used. See, e.g., Sambrook and Russell eds. (2001)Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure provides the first TCR mimic (TCRm) antibody directed to a cancer associated public phosphopeptide neoAg pIRS2 in complex with HLA-A2. Phosphopeptides are emerging as a new class of cancer-specific neoantigens, which appear with dysregulated kinase activity in cancer cells. Two key features that set phosphopeptides apart from other tumor associated antigens are: 1) TCR reactive with the phosphopeptides can discriminate them from their unphosphorylated wild type counterparts, thus, providing an increased level of cancer specificity and 2) phosphopeptides are public neoAgs expressed in a large number of different cancers, making them distinct from patient- and tumor-specific, mutation-derived neoAgs.

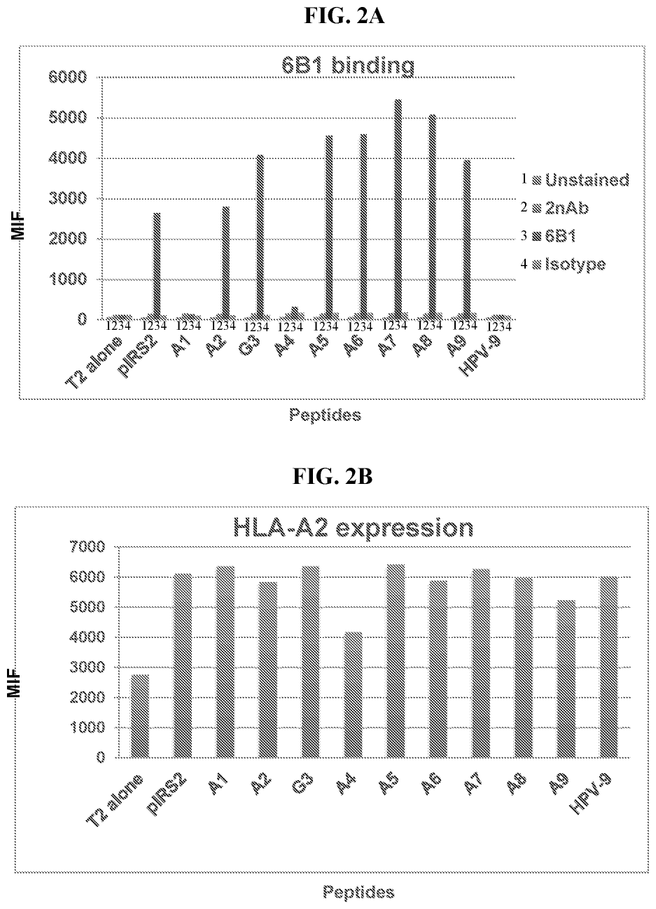

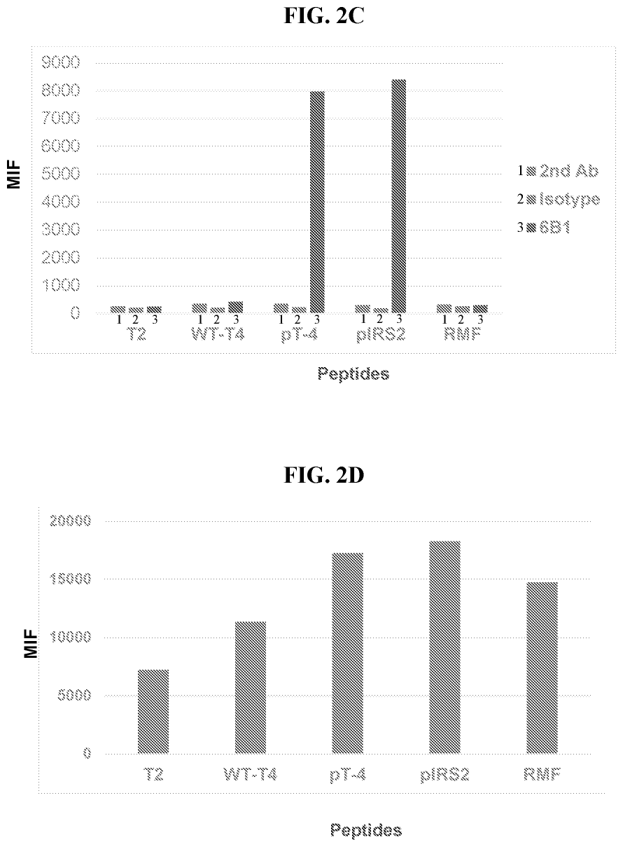

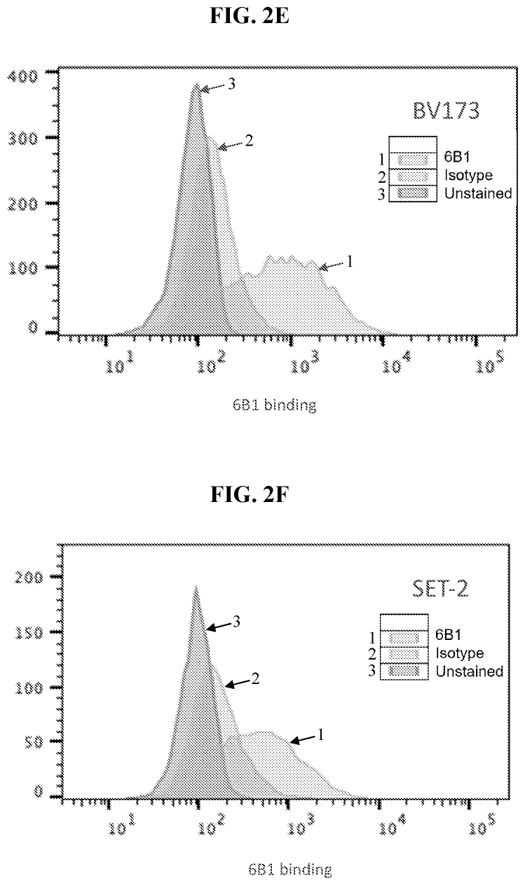

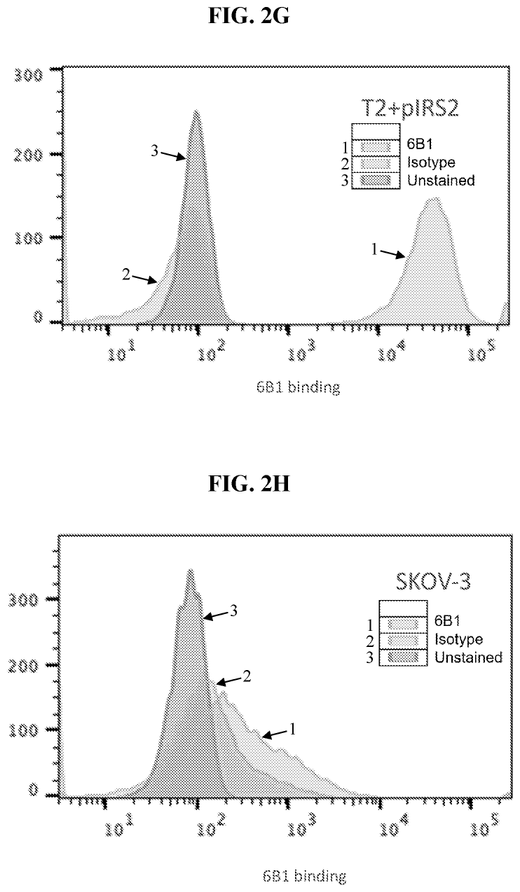

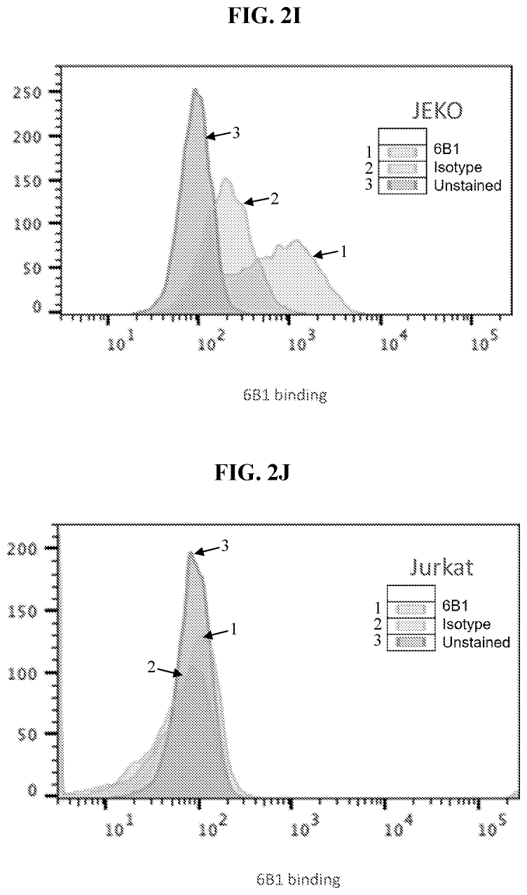

Since antigenic density of peptide/MHC complex on cell surface is typically 100 to 1000-fold lower than protein targets for conventional mAbs (Dao T et al., Sci Transl. Med. 2013; 5 (176): 176ra33; Chang A Y et al, J Clin Invest. 2017; 127(9): 3557), low antigen binding is expected. However, the present disclosure demonstrates that the TCRm monoclonal antibodies (mAbs) (e.g., 6B1) described herein were able to detect naturally presented pIRS2 epitope on tumor cells, and recognized the position 4 phosphoserine moiety, and the arginine residue at position 1 in the pIRS2 peptide RVA[pS]PTSGVK (SEQ ID NO: 19). Since arginine at pos1 and p-Ser at pos 4 are two crucial residues for epitope recognition, it is anticipated that 6B1 may also recognize other phosphopeptide/HLA-A2 complexes homologous at these two residues at pos 1 and pos 4, which were documented with analog peptides (see FIG. 2).

Phosphopeptide-induced T cell responses do not recognize the unphosphorylated epitopes. Similarly, the TCRm mAbs disclosed herein did not recognize wild type IRS2, thus discriminating against non-phosphorylated form of IRS2 and other putative non-phosphorylated peptides with similar compositions. Furthermore, the TCRm mAbs disclosed herein did not recognize several normal human primary cells. Moreover, bispecific full length mAb formats (e.g., 1+1 and H2+2) including the TCRm mAb sequences can effectively enhance T cell cytotoxicity against these low-density tumor targets.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another.

An “antigen-binding protein” is a protein or polypeptide that comprises an antigen-binding region or antigen-binding portion, that has a strong affinity to another molecule to which it binds. Antigen-binding proteins encompass antibodies, antibody fragments, chimeric antibody-T cell receptors (caTCRs), chimeric antigen receptors (CARs) and fusion proteins, and conjugates thereof.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

More particularly, an antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds pIRS2/MHC complex will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). An antibody or antigen binding fragment thereof specifically binds to an antigen.

As used herein, the term “antibody moiety” encompasses full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as 1gG1 (γ1 heavy chain), 1gG2 (γ2 heavy chain), 1gG3 (γ3 heavy chain), 1gG4 (γ4 heavy chain), 1gA1 (α1 heavy chain), or 1gA2 (α2 heavy chain).

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multispecific antibodies formed from antibody fragments.

“Bispecific antibody” or “BsAb”, as used herein, refers to an antibody that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including V_{H} and/or V_{L} regions from different monoclonal antibodies. In some embodiments, the bispecific antibody contains two antigen binding moieties, wherein one of the two antigen binding moieties includes an immunoglobulin molecule having V_{H} and/or V_{L} regions that contain CDRs from a first monoclonal antibody, and the other antigen binding moiety includes an antibody fragment (e.g., Fab, F(ab′), F(ab′)₂, Fd, Fv, dAB, scFv, etc.) having V_{H} and/or V_{L} regions that contain CDRs from a second monoclonal antibody.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fᵥ (scFv)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an immunoglobulin-related composition (e.g., antibody or antigen binding fragment thereof) can selectively bind. The target antigen may be a protein, carbohydrate, nucleic acid, lipid, hapten, or other naturally occurring or synthetic compound. In some embodiments, the target antigen may be a peptide/MHC complex (e.g., a pIRS2 peptide/MHC complex, also called “pIRS2/MHC complex”, “pIRS2 peptide-MHC complex”, “pIRS2-MHC complex”, or “pIRS2 peptide complexed with MHC” as used herein). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

By “binding affinity” is meant the strength of the total noncovalent interactions between a single binding site of a molecule (e.g., an immunoglobulin-related composition) and its binding partner (e.g., an antigen). The affinity of a molecule X for its partner Y can generally be represented by the dissociation constant (K_{D}). Affinity can be measured by standard methods known in the art, including those described herein. A low-affinity complex contains an immunoglobulin-related composition that generally tends to dissociate readily from the antigen, whereas a high-affinity complex contains an immunoglobulin-related composition that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “chimeric antibody-T cell receptors (caTCRs)” refers to a functional polypeptide complex typically comprising at least two separate polypeptide chains. The caTCR complex comprises an antigen-binding module that specifically binds to a target antigen and a T cell receptor module (TCRM). The TCRM typically comprises a first TCR domain (TCRD) on one of the two polypeptide chains, comprising a first TCR transmembrane domain (TCR-TM), and a second TCRD on the other polypeptide chain, comprising a second TCR-TM, wherein the TCRM facilitates recruitment of at least one TCR-associated signaling molecule, e.g., CD3ζ, upon specific binding of the antigen-binding module to its target antigen. The antigen-binding module of a caTCR complex is derived from an antibody, and caTCR complexes typically do not comprise an antigen-binding TCR variable domain or TCR CDR sequences. The caTCR complex itself does not comprise a functional primary immune cell signaling sequence, such as a functional signaling sequence comprising an ITAM, e.g., the intracellular domain of CD3ζ, CD3γ, CD3δ, CD3ε, FcRγ, FcRβ, CD5, CD22, CD79a, CD79b, or CD66d. In some embodiments, the caTCR complex itself does not comprise any primary immune cell signaling sequence,

In some embodiments, the antigen-binding module in caTCR comprises the anti-pIRS2/MHC antibody moiety described herein. In some embodiments, the antigen-binding module comprises a Fab, a Fab′, a F(ab′)₂, an Fv, or a single chain Fv (scFv). In some embodiments, the antigen-binding module comprises at least one scFv on one of the two polypeptide chains. In some embodiments, the antigen-binding module comprises at least one scFv on each of the two polypeptide chains. In some embodiments, the antigen-binding module comprises at least one Fab composed of an antibody heavy chain variable region (V_{H}) on one of the two polypeptide chains and an antibody light chain variable region (V_{L}) on the other polypeptide chain.

One of the TCR-TMs or TCRDs can be derived from or is a fragment of a TCR chain selected from TCRα, TCRβ, TCRγ, and TCRδ, while the other TCR-TM or TCRD can be derived from or is a fragment of another TCR chain selected from TCRα, TCRβ, TCRγ, and TCRδ which pairs with the first TCR fragment, e.g., TCRα-TCRβ pairing or TCRγ-TCRδ pairing.

In some embodiments, one of the two polypeptide chains comprises a V_{H} fused directly or indirectly to one of the TCRD (which can be called “the first TCRD”), while the other polypeptide chain comprises a V_{L} fused directly or indirectly to the other TCRD (which can be called “the second TCRD”). In some embodiments, there is a linker between V_{H} and the first TCRD and/or between V_{L} and the second TCRD. In some embodiments, the linker between V_{H} and the first TCRD can be CH1, CH2, CH3, CH4, or a TCR constant domain (which can be called “the first TCR constant domain), while the linker between V_{L} and the second TCRD can be CL (which pairs with CH1), CH2 (which pairs with CH2), CH3 (which pairs with CH3), CH4 (which pairs with CH4), or a TCR constant region that pairs with the first TCR constant domain, respectively. In some embodiments, the two polypeptide chains in a caTCR complex are linked by at least one disulfide bond.

In some embodiments, the caTCR does not include a functional co-stimulatory signaling sequences (e.g., the intracellular domain of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, or the like). In some embodiments, the caTCR does not include any co-stimulatory signaling sequences.

The terms “caTCR” and “antibody-TCR chimeric molecule or construct (abTCR)” are used interchangeably. Further descriptions and examples of caTCR and abTCR may be found in, e.g., WO2017/070608, filed Oct. 21, 2016 and WO2018/200582, filed Apr. 24, 2018, which are incorporated by reference herein in its entirety.

“Chimeric antigen receptor (CAR)” as used herein refers to an artificially constructed hybrid single-chain protein or single-chain polypeptide containing a single-chain variable fragment (scFv) as a part of the extracellular antigen-binding domain, linked to a transmembrane domain (e.g., the transmembrane domain of a co-stimulatory molecule such as CD28 or CD8), which is in turn linked to an intracellular immune cell (e.g., T cell or NK cell) signaling domain which comprises at least a functional primary immune cell signaling sequence. Primary immune cell signaling sequences act in a stimulatory manner and contain signaling motifs which are known as immunoreceptor tyrosine-based activation motifs or ITAMs. Examples of ITAM-containing primary immune cell signaling sequences include those derived from CD3ζ (a.k.a. TCRζ), FcRγ, FcRβ, CD3γ, CD3δ, CD3ε, CD5, CD22, CD79a, CD79b, and CD66d. A “functional” primary immune cell signaling sequence is a sequence that is capable of transducing an immune cell activation signal when operably coupled to an appropriate receptor. “Non-functional” primary immune cell signaling sequences, which may comprise fragments or variants of primary immune cell signaling sequences, are unable to transduce an immune cell activation signal. Compositions of the present technology, including single chain variable fragments (scFv), may be used for the preparation of chimeric antigen receptors, the preparation and use of which is generally known in the art.

There are currently three generations of CARs. The “first generation” CARs are typically single-chain polypeptides composed of a scFv as the antigen-binding domain fused to a transmembrane domain fused to cytoplasmic/intracellular domain of the T cell receptor (TCR) chain. The “first generation” CARs typically have the intracellular domain from the CD3ζ chain, which is the primary transmitter of signals from endogenous TCRs through TCR complexes which comprise TCRs and CD3 molecules. The “first generation” CARs can provide de novo antigen recognition and cause activation of both CD4+ and CD8+ T cells through their CD3ζ chain signaling domain in a single fusion molecule, independent of HLA-mediated antigen presentation.

The “second generation” CARs add intracellular domains from various co-stimulatory molecules (e.g., CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, or the like) to the cytoplasmic tail of the CAR to provide additional signals to the T cell. “Second generation” CARs comprise those that provide both co-stimulation (e.g., CD28 or 4-IBB) and activation (e.g., CD3ζ). Preclinical studies have indicated that the “second generation” CARs can improve the antitumor activity of T cells. For example, robust efficacy of the “second generation” CAR modified T cells was demonstrated in clinical trials targeting the CD19 molecule in patients with chronic lymphoblastic leukemia (CLL) and acute lymphoblastic leukemia (ALL).

The “third generation” CARs have multiple intracellular domains from various co-stimulatory molecules (e.g., from both CD28 and 4-1BB).

Benefits of caTCRs and CARs include their abilities to redirect immune cell (e.g., T cell or NK cell) specificity and reactivity toward a selected target in either MHC-restricted (in case of TCR-mimic antibodies) or non-MHC-restricted (in case of antibodies against cell surface proteins) manners, exploiting the antigen-binding properties of monoclonal antibodies.

As used herein, the term “consensus FR” means a framework (FR) antibody region in a consensus immunoglobulin sequence. The FR regions of an antibody do not contact the antigen.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens.

As used herein, the term “epitope” means an antigenic determinant capable of specific binding to an immunoglobulin-related composition such as an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents. In some embodiments, an “epitope” is a region of the target antigen to which the anti-pIRS2/MHC immunoglobulin-related compositions of the present technology specifically bind. In some embodiments, the epitope is a conformational epitope or a non-conformational epitope. To screen for anti-pIRS2/MHC immunoglobulin-related compositions which bind to an epitope, a routine cross-blocking assay such as that described in Antibodies, A Laboratory Manual, Cold Spring Harbor Laboratory, Ed Harlow and David Lane (1988), can be performed. This assay can be used to determine if an anti-pIRS2/MHC antibody binds the same site or epitope as an anti-pIRS2/MHC antibody of the present technology. Alternatively, or additionally, epitope mapping can be performed by methods known in the art. For example, the antibody sequence can be mutagenized such as by alanine scanning, to identify contact residues. In a different method, MHC complexes including peptides corresponding to different regions of pIRS2 protein can be used in competition assays with the test antibodies or with a test antibody and an antibody with a characterized or known epitope.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

The term “HLA-A2”, as used herein, representatively refers to the subtypes, examples of which include, but are not limited to, HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28 and HLA-A*02:50.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the term “hypervariable region” refers to the amino acid residues of an antibody which are responsible for antigen-binding. The hypervariable region generally comprises amino acid residues from a “complementarity determining region” or “CDR.” As used herein, the term “CDR” or “complementarity determining region” of an antibody (or immunoglobulin) is intended to mean the non-contiguous antigen combining sites found within the variable region of both heavy and light chain polypeptides. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

TABLE 1

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an immunoglobulin-related composition described herein or amino acid sequence of an immunoglobulin-related composition described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an immunoglobulin-related composition) which recognizes and binds another molecule (e.g., an antigen), but that does not substantially recognize and bind other molecules. The terms “specific binding,” “specifically binds to,” or is “specific for” a particular molecule, as used herein, can be exhibited, for example, by a molecule having a K_{D} for the molecule to which it binds to of about 10⁻⁴M, 10⁻⁵M, 10⁻⁶M, 10⁻⁷M, 10⁻⁸M, 10⁻⁹M, 10⁻¹⁰ M, 10⁻¹¹M, or 10⁻¹² M. The term “specifically binds” may also refer to binding where a molecule (e.g., an immunoglobulin-related composition) binds to a particular target molecule or complex (e.g., a pIRS2 peptide/MHC complex), without substantially binding to any other molecule or complex.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

The term “T cell receptor,” or “TCR,” refers to a heterodimeric receptor composed of αβ or γδ chains that pair on the surface of a T cell. Each α, β, γ, and δ chain is composed of two Ig-like domains: a variable domain (V) that confers antigen recognition through the complementarity determining regions (CDR), followed by a TCR constant domain (C) that is anchored to cell membrane by a connecting peptide and a transmembrane (TM) region. The TM region associates with the invariant subunits of the CD3 signaling apparatus. Each of the V domains has three TCR CDRs. These TCR CDRs interact with a complex between an antigenic peptide bound to a protein encoded by the major histocompatibility complex (pMHC) (Davis and Bjorkman (1988) Nature, 334, 395-402; Davis et al. (1998) Annu Rev Immunol, 16, 523-544; Murphy (2012), xix, 868 p.).

The term “TCR-associated signaling molecule” refers to a molecule having a cytoplasmic immunoreceptor tyrosine-based activation motif (ITAM) that is part of the TCR-CD3 complex. TCR-associated signaling molecules include CD3γε, CD3δε, and CD3ζ (also known as ζζ, CD3ζζ, or TCRζ).

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-pIRS2/MHC immunoglobulin-related compositions described herein are contemplated. For example, it may be desirable to improve the binding affinity and/or other biological properties of an immunoglobulin-related composition disclosed herein. Amino acid sequence variants of an anti-pIRS2/MHC immunoglobulin-related composition are prepared by introducing appropriate nucleotide changes into its nucleic acid, or by peptide synthesis. Such modifications include, for example, deletions from, and/or insertions into and/or substitutions of, residues within the amino acid sequences of the immunoglobulin-related composition. Any combination of deletion, insertion, and substitution is made to obtain the immunoglobulin-related composition of interest, as long as the obtained immunoglobulin-related composition possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table 2 below.

TABLE 2
Amino Acid Substitutions

| | | Conservative |
|---|---|---|
| Original Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

One type of substitutional variant involves substituting one or more hypervariable region residues of a parent antibody. A convenient way for generating such substitutional variants involves affinity maturation using phage display. Specificity, several hypervariable region sites (e.g., 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen. Such contact residues and neighboring residues are candidates for substitution according to the techniques elaborated herein. Once such variants are generated, the pane of variants is subjected to screening as described herein and antibodies with similar or superior properties in one or more relevant assays may be selected for further development.

### Immunoglobulin-related Compositions of the Present Technology

“Immunoglobulin-related compositions” as used herein, refers to antibodies (including monoclonal antibodies, polyclonal antibodies, humanized antibodies, chimeric antibodies, human antibodies, recombinant antibodies, multispecific antibodies, bispecific antibodies, etc.), antibody fragments, chimeric antibody-T cell receptors (caTCRs), chimeric antigen receptors (CARs), fusion proteins, and conjugates thereof.

The present disclosure relates to immunoglobulin-related compositions that specifically recognize epitopes of a complex of a peptide/protein fragment derived from an intracellular pIRS2 protein, and an MHC class I molecule, for example, as the complex might be displayed at the cell surface during antigen presentation.

Traditionally, the MHC-peptide complex could only be recognized by a T-cell receptor (TCR), limiting the ability to detect an epitope of interest using T cell-based readout assays. The present technology describes methods and compositions for the generation and use of anti-pIRS2/MHC immunoglobulin-related compositions. In the present disclosure, immunoglobulin-related compositions, including antibodies, having an antigen-binding region based on scFvs that are selected from human scFv phage display libraries using recombinant HLA-peptide complexes are described. These molecules demonstrated specificity, for example as shown with anti-pIRS2/MHC antibodies that recognize only HLA-A*02-RVA[pS]PTSGVK (SEQ ID NO: 19) complexes. Accordingly, the immunoglobulin-related compositions of the present disclosure operate as “TCR mimic” antigen binding proteins. In addition, the molecules were also unable to bind HLA-complexes containing other peptides, further demonstrating their TCR-like specificity.

The anti-pIRS2/MHC immunoglobulin-related compositions of the present disclosure may be useful in the diagnosis, or treatment of pIRS2-associated diseases (e.g., cancers). Anti-pIRS2/MHC immunoglobulin-related compositions within the scope of the present technology include, e.g., but are not limited to, monoclonal antibodies, polyclonal antibodies, humanized antibodies, human antibodies (e.g., fully human antibodies), chimeric antibodies, recombinant antibodies, multispecific antibodies, bispecific antibodies, diabodies, antibody fragments (e.g., Fab, F(ab)′2, Fab′, scFv, and Fᵥ), chimeric antibody-T cell receptors (caTCRs), chimeric antigen receptors (CARs), fusion proteins, and conjugates that specifically bind the target antigen, a homolog, derivative or a fragment thereof. Additionally or alternatively, in some embodiments, the immunoglobulin-related compositions of the present technology may be an immunoglobulin polypeptide, or an immunoglobulin-like polypeptide.

Without wishing to be bound by theory, it is anticipated that the TCR mimic immunoglobulin-related compositions of the present disclosure simulate the function of a TCR in a T cell because like a TCR, the immunoglobulin-related compositions only recognize the target peptide RVA[pS]PTSGVK (SEQ ID NO: 19) when complexed with HLA-A*02.

Table 3 below provides V_{H} CDR sequences of the immunoglobulin-related compositions of present technology:

TABLE 3

| V_{H} CDR1 | V_{H} CDR2 | V_{H} CDR3 |
|---|---|---|
| GFTFSSYA | ISGGGGYT | ARQMYYYYGMDV |
| (SEQ ID NO: 20) | (SEQ ID NO: 21) | (SEQ ID NO: 22) |

Table 4 below provides V_{L} CDR sequences of the immunoglobulin-related compositions of present technology:

TABLE 4

| V_{L} CDR1 | V_{L} CDR2 | V_{L} CDR3 |
|---|---|---|
| QSVSANY | GAS | QQSYQRPLT |
| (SEQ ID NO: 23) | SEQ ID NO: 24) | (SEQ ID NO: 25) |

The V_{H} amino acid sequences of the pIRS2-specific clones of the present technology (SEQ ID NO: 17) are provided below. The V_{H} CDR 1-3 sequences are underlined.

(SEQ ID NO: 17)
EVQLLESGGGLVQPGGSLRLSCAASGFTFSSYAMSWVRQAPGKGLEWVS
EISGGGGYTDYADSVKGRFTISRDNSKNTLYLQMNSLRAEDTAVYYCAR
QMYYYYGMDVWGQVTTVTVSS

The V_{L} amino acid sequences of the pIRS2-specific clones of the present technology (SEQ ID NO: 18) are provided below. The V_{L} CDR 1-3 sequences are underlined.

(SEQ ID NO: 18)
EIVLTQSPGTLSLSPGERATLSCRASQSVSANYLAWYQQKPGQAPRLLI
YGASSRATGIPDRFSGSGSGTDFTLTISRLEPEDFAVYYCQQSYQRPLT
FGQGTKVEIK

In some embodiments, the present disclosure includes anti-pIRS2/MHC immunoglobulin-related compositions that have a scFv sequence fused to one or more constant domains of a heavy chain to form an antibody with an Fc region of a human immunoglobulin to yield a bivalent protein, increasing the overall avidity and stability of the antibody. In certain embodiments, the present disclosure includes anti-pIRS2/MHC immunoglobulin-related compositions that have a scFv sequence fused to one or more hinge domains of a heavy chain to form an antibody with an Fc region of a human immunoglobulin to yield a bivalent protein, increasing the overall avidity and stability of the antibody. The results presented here highlight the specificity, sensitivity and utility of the immunoglobulin-related compositions of the present disclosure in targeting MHC-peptide complexes.

The molecules of the present disclosure are based on the identification and selection of single chain variable fragments (scFv) using phage display, the amino acid sequence of which confers the molecules' specificity for the MHC restricted peptide of interest and forms the basis of all immunoglobulin-related compositions of the disclosure. The scFv, therefore, can be used to design a diverse array of “antibody” molecules, including, for example, full length antibodies, fragments thereof, such as Fab and F(ab′)₂, minibodies, fusion proteins, including scFv-Fc fusions, multivalent antibodies, that is, antibodies that have more than one specificity for the same antigen or different antigens, for example, bispecific T-cell engaging antibodies (BiTe), tribodies, etc. (see Cuesta et al., Multivalent antibodies: when design surpasses evolution. Trends in Biotechnology 28:355-362 2010).

In one aspect, the present disclosure provides immunoglobulin-related compositions comprising antibody moieties that specifically bind to pIRS2 peptide/MHC complexes (also called “anti-pIRS2 peptide/MHC” or “anti-pIRS2/MHC” herein). Such compositions can comprise, consist essentially of, or consist of, e.g., anti-pIRS2 peptide/MHC antibodies or antigen binding fragments thereof, chimeric antibody-T cell receptors (caTCRs), chimeric antigen receptors (CARs), fusion proteins, and conjugates. The antibody moieties can comprise: a heavy chain immunoglobulin variable domain (V_{H}) comprising a V_{H}-CDR1 sequence, a V_{H}-CDR2 sequence, and a V_{H}-CDR3 sequence of the V_{H} sequence of SEQ ID NO: 17, and a light chain immunoglobulin variable domain (V_{L}) comprising a V_{L}-CDR1 sequence, a V_{L}-CDR2 sequence, and a V_{L}-CDR3 sequence of the V_{L} sequence SEQ ID NO: 18.

Additionally or alternatively, in some embodiments of the immunoglobulin-related compositions (e.g., antibodies or antigen binding fragments thereof, chimeric antibody-T cell receptors (caTCRs), chimeric antigen receptors (CARs), fusion proteins, and conjugates) described herein, the V_{H}-CDR1 sequence comprises the sequence of SEQ ID NO: 20, the V_{H}-CDR2 sequence comprises the sequence of SEQ ID NO: 21, the V_{H}-CDR3 sequence comprises the sequence of SEQ ID NO: 22, the V_{L}-CDR1 sequence comprises the sequence of SEQ ID NO: 23, the V_{L}-CDR2 sequence comprises the sequence of SEQ ID NO: 24, and the V_{L}-CDR3 sequence comprises the sequence of SEQ ID NO: 25.

In one aspect, the present technology provides immunoglobulin-related compositions (e.g., antibodies or antigen binding fragments thereof, chimeric antibody-T cell receptors (caTCRs), chimeric antigen receptors (CARs), fusion proteins, and conjugates) comprising a heavy chain immunoglobulin variable domain (V_{H}) and a light chain immunoglobulin variable domain (V_{L}), wherein (a) the V_{H} comprises an amino acid sequence of SEQ ID NO: 17, or a variant thereof having one or more conservative amino acid substitutions; and/or (b) the V_{L} comprises an amino acid sequence of: SEQ ID NO: 18, or a variant thereof having one or more conservative amino acid substitutions.

In certain embodiments, the immunoglobulin-related composition includes one or more of the following characteristics: (a) a light chain immunoglobulin variable domain sequence that is at least 80%, at least 85%, at least 90%, at least 95%, or at least 99% identical to the light chain immunoglobulin variable domain sequence present in any one of SEQ ID NO: 18; and/or (b) a heavy chain immunoglobulin variable domain sequence that is at least 80%, at least 85%, at least 90%, at least 95%, or at least 99% identical to the heavy chain immunoglobulin variable domain sequence present in any one of SEQ ID NOs: 17. In another aspect, one or more amino acid residues in the immunoglobulin-related compositions provided herein are substituted with another amino acid. The substitution may be a “conservative substitution” as defined herein.

In any of the above embodiments, the immunoglobulin-related composition further comprises a Fc domain of any isotype, e.g., but are not limited to, IgG (including IgG1, IgG2, IgG3, and IgG4), IgA (including IgA1 and IgA2), IgD, IgE, or IgM, and IgY. Non-limiting examples of constant region sequences include:

Human IgD constant region, Uniprot: P01880
(SEQ ID NO: 26)
APTKAPDVFPIISGCRHPKDNSPVVLACLITGYHPTSVTVTWYMGTQSQPQRTFPEIQ
RRDSYYMTSSQLSTPLQQWRQGEYKCVVQHTASKSKKEIFRWPESPKAQASSVPTA
QPQAEGSLAKATTAPATTRNTGRGGEEKKKEKEKEEQEERETKTPECPSHTQPLGVY
LLTPAVQDLWLRDKATFTCFVVGSDLKDAHLTWEVAGKVPTGGVEEGLLERHSNG
SQSQHSRLTLPRSLWNAGTSVTCTLNHPSLPPQRLMALREPAAQAPVKLSLNLLASS
DPPEAASWLLCEVSGFSPPNILLMWLEDQREVNTSGFAPARPPPQPGSTTFWAWSVL
RVPAPPSPQPATYTCVVSHEDSRTLLNASRSLEVSYVTDHGPMK
Human IgG1 constant region, Uniprot: P01857
(SEQ ID NO: 27)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPR
EPQVYTLPPSRDELTKNQVSLTCLVKGFYPSDIA VEWESNGQPENNYKTTPPVLDSD
GSFFLYSKLTVDKSRWQQGNVFSCSVMHEALHNHYTQKSLSLSPGK
Human IgG2 constant region, Uniprot: P01859
(SEQ ID NO: 28)
ASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVY
TLPPSREEMTKNQVSLTCLVKGFYPSDISVEWESNGQPENNYKTTPPMLDSDGSFFL
YSKLTVDKSRWQQGNVFSCSVMHEALHNHYTQKSLSLSPGK
Human IgG3 constant region, Uniprot: P01860
(SEQ ID NO: 29)
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTPLGDTTHTCPRC
PEPKSCDTPPPCPRCPEPKSCDTPPPCPRCPEPKSCDTPPPCPRCPAPELLGGPSVFLFPP
KPKDTLMISRTPEVTCVVVDVSHEDPEVQFKWYVDGVEVHNAKTKPREEQYNSTFR
VVSVLTVLHQDWLNGKEYKCKVSNKALPAPIEKTISKTKGQPREPQVYTLPPSREEM
TKNQVSLTCLVKGFYPSDIA VEWESSGQPENNYNTTPPMLDSDGSFFLYSKLTVDKS
RWQQGNIFSCSVMHEALHNRFTQKSLSLSPGK
Human IgM constant region, Uniprot: P01871
(SEQ ID NO: 30)
GSASAPTLFPLVSCENSPSDTSSVAVGCLAQDFLPDSITLSWKYKNNSDISSTRGFPSV
LRGGKYAATSQVLLPSKDVMQGTDEHVVCKVQHPNGNKEKNVPLPVIAELPPKVSV
FVPPRDGFFGNPRKSKLICQATGFSPRQIQVSWLREGKQVGSGVTTDQVQAEAKESG
PTTYKVTSTLTIKESDWLGQSMFTCRVDHRGLTFQQNASSMCVPDQDTAIRVFAIPPS
FASIFLTKSTKLTCLVTDLTTYDSVTISWTRQNGEAVKTHTNISESHPNATFSAVGEAS
ICEDDWNSGERFTCTVTHTDLPSPLKQTISRPKGVALHRPDVYLLPPAREQLNLRESA
TITCLVTGFSPADVFVQWMQRGQPLSPEKYVTSAPMPEPQAPGRYFAHSILTVSEEE
WNTGETYTCVAHEALPNRVTERTVDKSTGKPTLYNVSLVMSDTAGTCY
Human IgG4 constant region, Uniprot: P01861
(SEQ ID NO: 31)
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
Human IgA1 constant region, Uniprot: P01876
(SEQ ID NO: 32)
ASPTSPKVFPLSLCSTQPDGNVVIACLVQGFFPQEPLSVTWSESGQGVTARNFPPSQD
ASGDLYTTSSQLTLPATQCLAGKSVTCHVKHYTNPSQDVTVPCPVPSTPPTPSPSTPP
TPSPSCCHPRLSLHRPALEDLLLGSEANLTCTLTGLRDASGVTFTWTPSSGKSAVQGP
PERDLCGCYSVSSVLPGCAEPWNHGKTFTCTAAYPESKTPLTATLSKSGNTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKPTHVNVSVV
MAEVDGTCY
Human IgA2 constant region, Uniprot: P01877
(SEQ ID NO: 33)
ASPTSPKVFPLSLDSTPQDGNVVVACLVQGFFPQEPLSVTWSESGQNVTARNFPPSQD
ASGDLYTTSSQLTLPATQCPDGKSVTCHVKHYTNPSQDVTVPCPVPPPPPCCHPRLSL
HRPALEDLLLGSEANLTCTLTGLRDASGATFTWTPSSGKSAVQGPPERDLCGCYSVS
SVLPGCAQPWNHGETFTCTAAHPELKTPLTANITKSGNTFRPEVHLLPPPSEELALNE
LVTLTCLARGFSPKDVLVRWLQGSQELPREKYLTWASRQEPSQGTTTFAVTSILRVA
AEDWKKGDTFSCMVGHEALPLAFTQKTIDRMAGKPTHVNVSVVMAEVDGTCY
Human Ig kappa constant region, Uniprot: P01834
(SEQ ID NO: 34)
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
Human Ig lambda constant 3, Uniprot: P0DOY3
(SEQ ID NO: 35)
GQPKAAPSVTLFPPSSEELQANKATLVCLISDFYPGAVTVAWKADSSPVKAGVETTT
PSKQSNNKYAASSYLSLTPEQWKSHKSYSCQVTHEGSTVEKTVAPTECS
Human Ig lambda constant 2, Uniprot: P0DOY2
(SEQ ID NO: 36)
GQPKAAPSVTLFPPSSEELQANKATLVCLISDFYPGAVTVAWKADSSPVKAGVETTT
PSKQSNNKYAASSYLSLTPEQWKSHRSYSCQVTHEGSTVEKTVAPTECS
Human Ig lambda constant 7, Uniprot: A0M8Q6
(SEQ ID NO: 37)
GQPKAAPSVTLFPPSSEELQANKATLVCLVSDFNPGAVTVAWKADGSPVKVGVETT
KPSKQSNNKYAASSYLSLTPEQWKSHRSYSCRVTHEGSTVEKTVAPAECS
Human Ig lambda constant 6, Uniprot: P0CF74
(SEQ ID NO: 38)
GQPKAAPSVTLFPPSSEELQANKATLVCLISDFYPGAVKVAWKADGSPVNTGVETTT
PSKQSNNKYAASSYLSLTPEQWKSHRSYSCQVTHEGSTVEKTVAPAECS
Human Ig lambda constant 1, Uniprot: P0CG04
(SEQ ID NO: 39)
GQPKANPTVTLFPPSSEELQANKATLVCLISDFYPGAVTVAWKADGSPVKAGVETTK
PSKQSNNKYAASSYLSLTPEQWKSHRSYSCQVTHEGSTVEKTVAPTECS

In some embodiments, the immunoglobulin-related compositions of the present technology comprise a heavy chain constant region that is at least 80%, at least 85%, at least 90%, at least 95%, at least 99%, or is 100% identical to SEQ ID NOs: 26-33. Additionally or alternatively, in some embodiments, the immunoglobulin-related compositions of the present technology comprise a light chain constant region that is at least 80%, at least 85%, at least 90%, at least 95%, at least 99%, or is 100% identical to SEQ ID NOs: 34-39. In some embodiments, the epitope is a conformational epitope or non-conformational epitope. Additionally or alternatively, in some embodiments, the immunoglobulin-related compositions of the present technology bind to an epitope comprising the amino acid sequence of SEQ ID NO: 19.

Additionally or alternatively, in some embodiments, the immunoglobulin-related compositions of the present technology comprise a Heavy Chain (HC) amino acid sequence and a Light chain (LC) amino acid sequence selected from the group consisting of: SEQ ID NO: 1 and SEQ ID NO: 2; SEQ ID NO: 4 and SEQ ID NO: 5; and SEQ ID NO: 6 and SEQ ID NO: 7.

Additionally or alternatively, in some embodiments, the immunoglobulin-related compositions of the present technology comprise a first Heavy Chain (HC), a second Heavy Chain (HC), and two identical Light Chains (LC), wherein the amino acid sequence of the first HC, the second HC and the LC are selected from the group consisting of: SEQ ID NO: 8, SEQ ID NO: 9, and SEQ ID NO: 10; SEQ ID NO: 11, SEQ ID NO: 12, and SEQ ID NO: 13; and SEQ ID NO: 14, SEQ ID NO: 15, and SEQ ID NO: 16.

In some embodiments in which the immunoglobulin-related composition is a full length antibody, the heavy and light chains of an antibody of the present disclosure may be full-length (e.g., an antibody can include at least one, or two, complete heavy chains, and at least one, or two, complete light chains) or may include an antigen-binding portion (a Fab, F(ab′)₂, Fv or a single chain Fv fragment (“scFv”)). In other embodiments, the antibody heavy chain constant region is chosen from, e.g., IgG1, IgG2, IgG3, IgG4, IgM, IgA1, IgA2, IgD, and IgE. In some embodiments, the immunoglobulin isotype is selected from IgG1, IgG2, IgG3, and IgG4, more particularly, IgG1 (e.g., human IgG1). In another embodiment, the antibody light chain constant region is chosen from, e.g., kappa or lambda, particularly human kappa or human lambda. The choice of antibody type will depend on the immune effector function that the antibody is designed to elicit. In constructing a recombinant immunoglobulin, appropriate amino acid sequences for constant regions of various immunoglobulin isotypes and methods for the production of a wide array of antibodies are known to those of skill in the art.

In any of the above embodiments of the immunoglobulin-related compositions, the V_{H} and V_{L} amino acid sequences form an antigen binding site that binds to a peptide:MHC complex (e.g., a pIRS2 peptide/MHC complex). In some embodiments, the epitope is a conformational epitope or a non-conformational epitope. In some embodiments, the V_{H} and V_{L} sequences are components of the same polypeptide chain. In other embodiments, the V_{H} and V_{L} sequences are components of different polypeptide chains. In certain embodiments, the immunoglobulin-related composition is a full-length antibody.

In some embodiments, the immunoglobulin-related compositions of the present technology bind specifically to at least one peptide/MHC complex (e.g., a pIRS2 peptide/MHC complex). In some embodiments, the immunoglobulin-related compositions of the present technology bind at least one peptide/MHC complex (e.g., a pIRS2 peptide/MHC complex) with a dissociation constant (K_{D}) of about 10⁻³M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In certain embodiments, the immunoglobulin-related compositions are monoclonal antibodies, chimeric antibodies, humanized antibodies, or bispecific antibodies. In some embodiments, the immunoglobulin-related compositions comprise a human antibody framework region.

In one aspect, the present disclosure provides an anti-pIRS2/MHC composition comprising an antibody moiety that competes with the any of the antigen binding proteins or immunoglobulin-related compositions of the present technology for specific binding to a pIRS2/MHC complex.

The immunoglobulin-related compositions of the present technology are intended to encompass bispecific antibodies, including bispecific T-cell engaging antibodies, that is, antibodies comprising two antibody variable domains on a single polypeptide chain that are able to bind two separate antigens. Where a first portion of a bispecific antibody binds an antigen on a tumor cell for example and a second portion of a bispecific antibody recognizes an antigen on the surface of a human immune effector cell, the antibody is capable of recruiting the activity of that effector cell by specifically binding to the effector antigen on the human immune effector cell. In some instances, bispecific antibodies, therefore, are able to form a link between effector cells, for example, T cells and tumor cells, thereby enhancing effector function. In one embodiment, the constant region/framework region is altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site). Furthermore, conjugation of the antibody to a drug, toxin, radioisotope, cytokine, inflammatory peptide or cytotoxic agent is also contemplated.

In some aspects, the anti-pIRS2/MHC immunoglobulin-related compositions described herein contain structural modifications to facilitate rapid binding and cell uptake and/or slow release. In some aspects, the anti-pIRS2/MHC immunoglobulin-related composition of the present technology (e.g., an antibody) may contain a deletion in the CH2 constant heavy chain region to facilitate rapid binding and cell uptake and/or slow release. In some aspects, a Fab fragment is used to facilitate rapid binding and cell uptake and/or slow release. In some aspects, a F(ab)′₂ fragment is used to facilitate rapid binding and cell uptake and/or slow release.

Additionally or alternatively, in some embodiments, the antigen binding proteins or immunoglobulin-related compositions of the present technology are chimeric antibody-T cell receptors (caTCR) and/or comprise at least a fragment of a T cell receptor (TCR) chain. In some embodiments, the fragment of TCR chain comprises the transmembrane domain of the TCR chain. In certain embodiments, the fragment of TCR chain does not comprise any CDR sequence of the TCR chain. Additionally or alternatively, in some embodiments, the antigen binding proteins or immunoglobulin-related compositions of the present technology are chimeric antigen receptors (CAR).

In any and all of the preceding embodiments, the antigen binding proteins or immunoglobulin-related compositions of the present technology may be monospecific, multispecific, or bispecific. In some embodiments, the antigen binding proteins or immunoglobulin-related compositions of the present technology comprise a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody. Additionally or alternatively, in some embodiments, the antigen binding proteins or immunoglobulin-related compositions of the present technology comprise a tandem scFv with at least one peptide linker between two scFvs. In certain embodiments, the antigen binding proteins or immunoglobulin-related compositions of the present technology comprise a second antibody moiety that specifically binds to a second antigen. The second antigen may be a disease-specific antigen that is not pIRS2/MHC, or an antigen on the surface of a T cell, a natural killer cell, a neutrophil, a monocyte, a macrophage, or a dendritic cell.

Additionally or alternatively, in some embodiments, the immunoglobulin-related compositions (e.g., antibodies, antigen binding fragments, chimeric antibody-T cell receptors (caTCRs), chimeric antigen receptors (CARs), fusion proteins, and conjugates thereof) specifically bind to a pIRS2 peptide complexed with HLA-A*02. The HLA-A*02 may be HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50. In certain embodiments, the pIRS2 peptide comprises the amino acid sequence RVA[pS]PTSGVK (SEQ ID NO: 19).

In one aspect, the present disclosure provides recombinant nucleic acids or a set of recombinant nucleic acids encoding any and all embodiments of the antigen binding proteins or immunoglobulin-related compositions described herein, with all components of the composition encoded by one nucleic acid or by the set of nucleic acids. In another aspect, the present disclosure provides a vector comprising said recombinant nucleic acids, as well as a set of vectors comprising said set of recombinant nucleic acids. Vectors comprising the nucleic acids of the present technology for antibody-based treatment by vectored immunotherapy are also contemplated by the present technology. Vectors include expression vectors which enable the expression and secretion of immunoglobulin-related compositions, such as antibodies, as well as vectors which are directed to cell surface expression of the immunoglobulin-related compositions, such as chimeric antigen receptors.

Also disclosed herein are cells comprising any of the recombinant nucleic acids, set of recombinant nucleic acids, vectors, or set of vectors disclosed herein, as well as cells that display on its surface or secrete any of the antigen binding proteins or immunoglobulin-related compositions of the present technology. The cells may be immune effector cells, such as a T cell, a NK cell, a B cell, or a monocyte/macrophage.

The immunoglobulin-related compositions of the present technology (e.g., an anti-pIRS2/MHC antibody) can be monospecific, bispecific, trispecific or of greater multispecificity. Multispecific antibodies can be specific for different epitopes of one or more peptide/MHC complexes (e.g., a pIRS2 peptide/MHC complex) or can be specific for both the pIRS2 peptide/MHC complexes as well as for heterologous compositions, such as a heterologous polypeptide or solid support material. See, e.g., WO 93/17715; WO 92/08802; WO 91/00360; WO 92/05793; Tutt et al., J. Immunol. 147: 60-69 (1991); U.S. Pat. Nos. 5,573,920, 4,474,893, 5,601,819, 4,714,681, 4,925,648; 6,106,835; Kostelny et al., J. Immunol. 148: 1547-1553 (1992). In some embodiments, the immunoglobulin-related compositions are chimeric. In certain embodiments, the immunoglobulin-related compositions are humanized.

The immunoglobulin-related compositions of the present technology can further be recombinantly fused to a heterologous polypeptide at the N- or C-terminus or chemically conjugated (including covalently and non-covalently conjugations) to polypeptides or other compositions. For example, the immunoglobulin-related compositions of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In certain embodiments, the Fc portion allows the direct conjugation of other molecules, including but not limited to fluorescent dyes, cytotoxins, radioisotopes etc. to the immunoglobulin-related composition for example, for use in antigen quantitation studies, to immobilize the immunoglobulin-related composition for affinity measurements, for targeted delivery of a therapeutic agent, to test for Fc-mediated cytotoxicity using immune effector cells and many other applications.

In any of the above embodiments of the immunoglobulin-related compositions of the present technology, the immunoglobulin-related compositions may be optionally conjugated to an agent selected from the group consisting of detectable labels, isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof. Conjugates of the immunoglobulin-related compositions of the present technology with therapeutic agents, include without limitation, drugs (such as calecheamicin, aureastatin, doxorubicin), or toxins (such as ricin, diphtheria, gelonin) or radioisotopes emitting alpha or beta particles (such as ⁹⁰Y, ¹³¹I, ²²⁵Ac, ²¹³Bi, ²²³Ra and ²²⁷Th), inflammatory peptides (such as IL2, TNF, IFN-γ) are encompassed by the present technology.

For a chemical bond or physical bond, a functional group on the immunoglobulin-related composition typically associates with a functional group on the agent. Alternatively, a functional group on the agent associates with a functional group on the immunoglobulin-related composition.

The functional groups on the agent and immunoglobulin-related composition can associate directly. For example, a functional group (e.g., a sulfhydryl group) on an agent can associate with a functional group (e.g., sulfhydryl group) on an immunoglobulin-related composition to form a disulfide. Alternatively, the functional groups can associate through a cross-linking agent (i.e., linker). Some examples of cross-linking agents are described below. The cross-linker can be attached to either the agent or the immunoglobulin-related composition. The number of agents or immunoglobulin-related compositions in a conjugate is also limited by the number of functional groups present on the other. For example, the maximum number of agents associated with a conjugate depends on the number of functional groups present on the immunoglobulin-related composition. Alternatively, the maximum number of immunoglobulin-related compositions associated with an agent depends on the number of functional groups present on the agent.

In yet another embodiment, the conjugate comprises one immunoglobulin-related composition associated to one agent. In one embodiment, a conjugate comprises at least one agent chemically bonded (e.g., conjugated) to at least one immunoglobulin-related composition. The agent can be chemically bonded to an immunoglobulin-related composition by any method known to those in the art. For example, a functional group on the agent may be directly attached to a functional group on the immunoglobulin-related composition. Some examples of suitable functional groups include, for example, amino, carboxyl, sulfhydryl, maleimide, isocyanate, isothiocyanate and hydroxyl.

The agent may also be chemically bonded to the immunoglobulin-related composition by means of cross-linking agents, such as dialdehydes, carbodiimides, dimaleimides, and the like. Cross-linking agents can, for example, be obtained from Pierce Biotechnology, Inc., Rockford, Ill. The Pierce Biotechnology, Inc. web-site can provide assistance. Additional cross-linking agents include the platinum cross-linking agents described in U.S. Pat. Nos. 5,580,990; 5,985,566; and 6,133,038 of Kreatech Biotechnology, B.V., Amsterdam, The Netherlands.

Alternatively, the functional group on the agent and immunoglobulin-related composition can be the same. Homobifunctional cross-linkers are typically used to cross-link identical functional groups. Examples of homobifunctional cross-linkers include EGS (i.e., ethylene glycol bis[succinimidylsuccinate]), DSS (i.e., disuccinimidyl suberate), DMA (i.e., dimethyl adipimidate.2HCl), DTSSP (i.e., 3,3′-dithiobis[sulfosuccinimidylpropionate])), DPDPB (i.e., 1,4-di-[3′-(2′-pyridyldithio)-propionamido]butane), and BMH (i.e., bis-maleimidohexane). Such homobifunctional cross-linkers are also available from Pierce Biotechnology, Inc.

In other instances, it may be beneficial to cleave the agent from the immunoglobulin-related composition. The web-site of Pierce Biotechnology, Inc. described above can also provide assistance to one skilled in the art in choosing suitable cross-linkers which can be cleaved by, for example, enzymes in the cell. Thus the agent can be separated from the immunoglobulin-related composition. Examples of cleavable linkers include SMPT (i.e., 4-succinimidyloxycarbonyl-methyl-a-[2-pyridyldithio]toluene), Sulfo-LC-SPDP (i.e., sulfosuccinimidyl 6-(3-[2-pyridyldithio]-propionamido)hexanoate), LC-SPDP (i.e., succinimidyl 6-(3-[2-pyridyldithio]-propionamido)hexanoate), Sulfo-LC-SPDP (i.e., sulfosuccinimidyl 6-(3-[2-pyridyldithio]-propionamido)hexanoate), SPDP (i.e., N-succinimidyl 3-[2-pyridyldithio]-propionamidohexanoate), and AEDP (i.e., 3-[(2-aminoethyl)dithio]propionic acid HCl).

In another embodiment, a conjugate comprises at least one agent physically bonded with at least one immunoglobulin-related composition. Any method known to those in the art can be employed to physically bond the agents with the immunoglobulin-related compositions. For example, the immunoglobulin-related compositions and agents can be mixed together by any method known to those in the art. The order of mixing is not important. For instance, agents can be physically mixed with immunoglobulin-related compositions by any method known to those in the art. For example, the immunoglobulin-related compositions and agents can be placed in a container and agitated, by for example, shaking the container, to mix the immunoglobulin-related compositions and agents.

The immunoglobulin-related compositions can be modified by any method known to those in the art. For instance, the immunoglobulin-related composition may be modified by means of cross-linking agents or functional groups, as described above.

The present technology is based on the identification of antigen-specific binding sequences from which a variety of immunoglobulin-related compositions can be produced. In addition to immunoglobulin-related compositions specific for an antigen that represents a protein fragment (peptide)/MHC complex similar to that typically recognized by a T-cell receptor following antigen processing and presentation of the protein to the T-cell, identification of amino acid and nucleic sequences as disclosed herein for the preparation of antibodies can also be used to generate other antigen-binding molecules including chimeric antigen receptors (CARs), with specificity for the protein fragment (peptide)/MHC complex. These can be incorporated into cells to make them specifically cytotoxic to the antigen expressing cell.

The present technology employs an approach to obtaining therapeutic antibodies to proteins that are inaccessible because they are not expressed on the cell surface. Nearly any intracytoplasmic or intranuclear protein (in addition to cell surface proteins) is a potential target for the approach described herein. This approach is to generate recombinant mAbs that recognize the peptide/MHC complex expressed on the cell surface, with the same specificity as a T-cell receptor (TCR). Such mAbs share functional homology with TCRs regarding target recognition, but confer higher affinity and capabilities of arming with potent cytotoxic agents that antibodies feature. TCR-like mAbs may be generated by conventional hybridoma techniques known to those of skill in the art, to produce human, humanized or chimeric antibodies. Recombinant antibodies with TCR-like specificity represent a valuable tool for research and therapeutic applications in tumor immunology and immunotherapy.

Further, fully-human mAbs may be used for therapeutic applications in humans because murine antibodies cause an immunogenicity reaction, known as the HAMA (human anti-mouse antibodies) response, when administered to humans, causing serious side effects, including anaphylaxis and hypersensitivity reactions. This immunogenicity reaction is triggered by the human immune system recognizing the murine antibodies as foreign because of slightly different amino acid sequences from natural human antibodies. Humanization methods known in the art can be employed to reduce the immunogenicity of murine-derived antibodies.

Recently, the use of phage display libraries has made it possible to select large numbers of Ab repertoires for unique and rare Abs against very defined epitopes (for more details on phage display see McCafferty et al., Phage antibodies: filamentous phage displaying antibody variable domains. Nature, 348: 552-554). The rapid identification of human Fab or single chain Fv (scFv) fragments highly specific for tumor antigen-derived peptide-MHC complex molecules has thus become possible. More recently, immuno-toxins, generated by fusing TCR-like Fab specific for melanoma Ag MART-1 26-35/A2 or gp100 280-288/A2 to a truncated form of Pseudomonas endotoxin, have been shown to inhibit human melanoma growth both in vitro and in vivo. In addition, by engineering full-length mAb using the Fab fragments, it is possible to directly generate a therapeutic human mAb, bypassing months of time-consuming work normally needed for developing therapeutic mAbs. The present technology involves the development of a TCR-like, human mAb that recognizes, for example, the pIRS2 peptide/HLA-A*02 complex (RVA[pS]PTSGVK (SEQ ID NO: 19)) for cancer therapy.

Identification of Peptides with High Predictive Binding to HLA Molecules. In some embodiments, the present technology relates to a method for the generation of antibodies that specifically bind to HLA-restricted peptides, which, when presented as part of a peptide/MHC complex are able to elicit a specific cytotoxic T-cell response. HLA class I molecules present endogenous derived peptides of about 8-12 amino acids in length to CD8⁺ cytotoxic T lymphocytes. Peptides to be used in the method of the present technology are generally about 6-22 amino acids in length, and in some embodiments, between about 9 and 20 amino acids and comprise an amino acid sequence derived from a protein of interest, for example, human IRS2 protein (Uniprot Accession No. Q9Y4H2) or an analog thereof.

Peptides suitable for use in generating antibodies in accordance with the method of the present technology can be determined based on the presence of HLA-A*02-binding motifs and the cleavage sites for proteasomes and immune-proteasomes using computer prediction models known to those of skill in the art. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), SYFPEITHI database (see Schuler et al. SYFPEITHI, Database for Searching and T-Cell Epitope Prediction. in Immunoinformatics Methods in Molecular Biology, vol 409(1): 75-93 2007), and netMHCpan 4.1 (see Birkir Reynisson et al., NetMHCpan-4.1 and NetMHCIIpan-4.0: Improved predictions of MHC antigen presentation by concurrent motif deconvolution and integration of MS MHC eluted ligand data. Nucleic Acids Research, 48(W1):W449-W454 (2020)). HLA-A*02:01 is expressed in 39-46% of all caucasians and therefore, represents a suitable choice of MHC antigen for use in the present method.

Once appropriate peptides have been identified, peptide synthesis may be done in accordance with protocols well known to those of skill in the art. Because of their relatively small size, the peptides of the present technology may be directly synthesized in solution or on a solid support in accordance with conventional peptide synthesis techniques. Various automatic synthesizers are commercially available and can be used in accordance with known protocols. The synthesis of peptides in solution phase has become a well-established procedure for large-scale production of synthetic peptides and as such is a suitable alternative method for preparing the peptides of the present technology. See for example, Solid Phase Peptide Synthesis by John Morrow Stewart and Martin et al. Application of Almez-mediated Amidation Reactions to Solution Phase Peptide Synthesis, Tetrahedron Letters Vol. 39, pages 1517-1520 1998).

Each of the peptides used in the protocols described herein may be synthesized using fluorenylmethoxycarbonyl chemistry and solid-phase synthesis and purified by high-pressure liquid chromatography. The quality of the peptides can be assessed by high-performance liquid chromatography analysis, and the expected molecular weight can be observed using matrix-assisted laser desorption mass spectrometry. Peptides are preferably sterile and 70% to 90% pure. The peptides may be dissolved in DMSO and diluted in PBS (pH 7.4) or saline at 5 mg/mL and stored at −80° C.

Subsequent to peptide selection, binding activity of selected peptides is tested using the antigen-processing-deficient T2 cell line, which increases expression of HLA-A when stabilized by a peptide in the antigen-presenting groove. Briefly, T2 cells are pulsed with peptide for a time sufficient to induce HLA-A expression. HLA-A expression of T2 cells is then measured by immunostaining with a fluorescently labeled monoclonal antibody specific for HLA-A (for example, BB7.2) and flow cytometry. Fluorescence index (FI) is calculated as the mean fluorescence intensity (MFI) of HLA-A*02 on T2 cells as determined by fluorescence-activated cell-sorting analysis, using the formula FI=(MFI [T2 cells with peptide]/MFI [T2 cells without peptide]−1.

Fully human T-cell receptor (TCR)-like antibodies to pIRS2 are produced using the method disclosed herein. TCR-like anti-pIRS2/MHC antibodies generated by phage display technology are specific for a pIRS2 peptide/HLA complex similar to that which induces HLA-restricted cytotoxic CD8 T-cells. The pIRS2 protein sequence may be screened using the netMHCpan 4.1 algorithm and pIRS2 peptides may be identified that had predicted high-affinity binding to multiple HLA molecules that are highly expressed in the Caucasian population.

Heteroclitic peptides can also be designed by conservative amino acid substitutions of MHC-binding residues expected to enhance the affinity toward the MHC class 1 allele, as predicted by the prediction algorithm. Peptides used for alanine mutagenesis of pIRS2 are named based on the position where the substitution was made. Examples of pIRS2 peptides which may be used include SEQ ID NO: 19. Once a suitable peptide has been identified, the target antigen to be used for phage display library screening, that is, a peptide/HLA complex (for example, pIRS2 peptide/HLA-A*02) is prepared by bringing the peptide and the histocompatibility antigen together in solution to form the complex.

Selecting a High Affinity ScFv Against a pIRS2 Peptide. The next step is the selection of phage that bind to the target antigen of interest with high affinity, from phage in a human phage display library that either do not bind or that bind with lower affinity. This is accomplished by iterative binding of phage to the antigen, which is bound to a solid support, for example, beads or mammalian cells followed by removal of non-bound phage and by elution of specifically bound phage. In certain embodiments, antigens are first biotinylated for immobilization to, for example, streptavidin-conjugated Dynabeads M-280. The phage library is incubated with the cells, beads or other solid support and non binding phage is removed by washing. Clones that bind are selected and tested.

Once selected, positive scFv clones are tested for their binding to HLA-A*02/peptide complexes on live T2 cell surfaces by indirect flow cytometry. Briefly, phage clones are incubated with T2 cells that have been pulsed with a pIRS2 peptide, or an irrelevant peptide (control). The cells are washed and then with a mouse anti-M13 coat protein mAb. Cells are washed again and labeled with a FITC-goat (Fab)₂ anti-mouse Ig prior to flow cytometry.

In other embodiments, the anti-pIRS2/MHC antibodies may comprise one or more framework region amino acid substitutions designed to improve protein stability, antibody binding, expression levels or to introduce a site for conjugation of therapeutic agents. These scFvs are then used to produce recombinant human monoclonal Igs in accordance with methods known to those of skill in the art.

### Uses of the Immunoglobulin-Related Compositions of the Present Technology

Methods for reducing the proliferation of leukemia cells and/or progression of the tumor or pathologic condition are also included, comprising contacting leukemia cells with an immunoglobulin-related composition of the present technology. Progression includes, e.g, the growth, invasiveness, metastases and/or recurrence of the tumor or pathologic condition. In a related aspect, the immunoglobulin-related compositions of the present technology can be used for the prevention or treatment of pIRS2-associated diseases (e.g., cancers). Such treatment can be used in patients identified as having pathologically high levels of pIRS2 (e.g., those diagnosed by the methods described herein) or in patients diagnosed with a disease known to be associated with such pathological levels.

In one aspect, the present disclosure provides a method for treating a pIRS2-associated disease in a subject in need thereof, comprising administering to the subject an effective amount of a composition comprising an antibody moiety that specifically binds to a pIRS2 peptide/HLA-A*02 complex.

In another aspect, the present disclosure provides a method for treating a pIRS2-associated disease in a subject in need thereof, comprising administering to the subject an effective amount of a recombinant nucleic acid, a set of recombinant nucleic acids, a vector, or a set of vectors that encode a composition comprising an antibody moiety that specifically binds to a pIRS2 peptide/HLA-A*02 complex.

In yet another aspect, the present disclosure provides a method for treating a pIRS2-associated disease in a subject in need thereof, comprising administering to the subject an effective amount of a pharmaceutical composition comprising a pharmaceutically-acceptable carrier and (a) a composition comprising an antibody moiety that specifically binds to a pIRS2 peptide/HLA-A*02 complex; or (b) a recombinant nucleic acid, a set of recombinant nucleic acids, a vector, or a set of vectors encoding the composition of (a); or (c) a cell comprising the recombinant nucleic acid, the set of recombinant nucleic acids, the vector, or the set of vectors of (b); or (d) a cell that displays on its surface or secretes the composition of (a). In certain embodiments, the cell of (c) or (d) is a T cell, a NK cell, a B cell, or a monocyte/macrophage.

In one aspect, the present disclosure provides methods for treating a pIRS2-associated disease in a subject in need thereof, comprising administering to the subject an effective amount of any immunoglobulin-related composition of the present technology.

In another aspect, the present disclosure provides methods for treating a pIRS2-associated disease in a subject in need thereof, comprising administering to the subject an effective amount of any of the recombinant nucleic acids, set of recombinant nucleic acids, vectors, set of vectors, or cells of the present technology.

In yet another aspect, the present disclosure provides methods for treating a pIRS2-associated disease in a subject in need thereof, comprising administering to the subject an effective amount of any of the pharmaceutical compositions disclosed herein.

Additionally or alternatively, in some embodiments of the methods disclosed herein, the pIRS2 peptide comprises the amino acid sequence RVA[pS]PTSGVK (SEQ ID NO: 19). In any and all embodiments of the methods disclosed herein, said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

In any and all embodiments of the methods disclosed herein, the pIRS2-associated disease is a cancer. Examples of cancers that can be treated by the immunoglobulin-related compositions of the present technology include any cancer presenting the RVA[pS]PTSGVK (SEQ ID NO: 19) peptide in complex with HLA-A*02, including but not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, ovarian cancer, breast cancer, cervical cancer, prostate cancer, melanoma, mesothelioma, pancreatic cancer, thyroid cancer, liver cancer, and hepatocellular carcinoma.

The compositions of the present technology may be employed in conjunction with other therapeutic agents useful in the treatment of pIRS2-associated diseases (e.g., cancers). For example, the immunoglobulin-related compositions of the present technology may be separately, sequentially or simultaneously administered with at least one additional therapeutic agent. Examples of additional therapeutic agents include, but are not limited to, antiangiogenic agents, alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

Other examples of additional therapeutic agents include, but are not limited to, immune checkpoint inhibitors, monoclonal antibodies that specifically target tumor antigens, cell-mediated immunotherapy (e.g., T cell therapy), immune activating agents (e.g., interferons, interleukins, cytokines), oncolytic virus therapy and cancer vaccines. Examples of immune checkpoint inhibitors include immuno-modulating/stimulating antibodies such as an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, and an anti-LAG-3 antibody. Specific immuno-modulating/stimulating antibodies include ipilimumab, Nivolumab, Pembrolizumab, Atezolizumab, Avelumab, and Durvalumab. Additionally or alternatively, in some embodiments, the monoclonal antibodies that specifically target tumor antigens bind to one or more targets selected from among CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, P1GF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Le^{y}) antigen, E-cadherin, V-cadherin, GPC3, EpCAM, DLL3, PD-1, PD-L1, CD28, CD137, CD99, GloboH, CD24, STEAP1, B7H3, Polysialic Acid, OX40, OX40-ligand, or other peptide MHC complexes (e.g., with peptides derived from TP53, KRAS, MYC, EBNA1-6, PRAME, MART, tyronsinase, MAGEA1-A6, pmel17, LMP2, or WT1). Examples of immune activating agents include, but are not limited to, interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

The compositions of the present technology may optionally be administered as a single bolus to a subject in need thereof. Alternatively, the dosing regimen may comprise multiple administrations performed at various times after the appearance of tumors.

Administration can be carried out by any suitable route, including orally, intranasally, parenterally (intravenously, intramuscularly, intraperitoneally, or subcutaneously), rectally, intracranially, intratumorally, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved. A clinician skilled in the art can readily determine, for example, by the use of clinical tests, physical examination and medical/family history, if an individual is a candidate for such treatment.

In accordance with the methods of the present disclosure, at least one pIRS2 immunoglobulin-related composition described herein is used to promote a positive therapeutic response to a pIRS2-associated disease (e.g., cancer). By “positive therapeutic response” is intended any improvement in the disease conditions associated with the activity of the immunoglobulin-related compositions of the present technology, and/or an improvement in the symptoms associated with the disease. Thus, for example, an improvement in the disease can be characterized as a complete response. By “complete response” is intended an absence of clinically detectable disease with normalization of any previously test results. Such a response can in some cases persist, e.g., for at least one month following treatment according to the methods of the present technology. Alternatively, an improvement in the disease can be categorized as being a partial response.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, ELISPOT, RIA, chromatography, and the like. In addition to these positive therapeutic responses, the subject undergoing therapy with the immunoglobulin-related compositions described herein can experience the beneficial effect of an improvement in the symptoms associated with the disease.

In some embodiments, the immunoglobulin-related compositions of the present technology comprise pharmaceutical formulations which may be administered to subjects in need thereof in one or more doses. Dosage regimens can be adjusted to provide the desired response (e.g., a therapeutic response).

Typically, an effective amount of the immunoglobulin-related compositions of the present technology, sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of anti-pIRS2/MHC immunoglobulin-related compositions, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks. In one embodiment, a single dosage of immunoglobulin-related composition ranges from 0.1-10,000 micrograms per kg body weight. In one embodiment, immunoglobulin-related composition concentrations in a carrier range from 0.2 to 2000 micrograms per delivered milliliter. An exemplary treatment regime entails administration once per every two weeks or once a month or once every 3 to 6 months. Anti-pIRS2/MHC immunoglobulin-related compositions may be administered on multiple occasions. Intervals between single dosages can be hourly, daily, weekly, monthly or yearly. Intervals can also be irregular as indicated by measuring blood levels of the immunoglobulin-related composition in the subject. In some methods, dosage of the immunoglobulin-related composition is adjusted to achieve a serum concentration in the subject of from about 75 μg/mL to about 125 μg/mL, 100 μg/mL to about 150 μg/mL, from about 125 μg/mL to about 175 μg/mL, or from about 150 μg/mL to about 200 μg/mL. Alternatively, anti-pIRS2/MHC immunoglobulin-related compositions can be administered as a sustained release formulation, in which case less frequent administration is required. Dosage and frequency vary depending on the half-life of the immunoglobulin-related composition in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In one aspect, the present disclosure provides a method for detecting expression levels of pIRS2 in a sample includes (a) contacting said sample with any of the immunoglobulin-related compositions described herein (e.g., antibodies such as human, humanized, or chimeric antibodies, antibody fragments, chimeric antibody-T cell receptors (caTCRs), chimeric antigen receptors (CARs), fusion proteins, and conjugates thereof) and (b) detecting binding to a pIRS2 peptide-HLA-A*02 complex in the biological sample. In some embodiments, the pIRS2 peptide comprises the amino acid sequence RVA[pS]PTSGVK (SEQ ID NO: 19). In another aspect, the present disclosure provides a method for detecting pIRS2 peptides on the surface of cells or tissues using any of the immunoglobulin-related compositions of the present disclosure (e.g., antibodies such as humanized, chimeric or human antibodies, antibody fragments, chimeric antibody-T cell receptors (caTCRs), chimeric antigen receptors (CARs), fusion proteins, and conjugates thereof). Methods for detecting peptide or protein expression are well known in the art and include, but are not limited to, PCR techniques, immunohistochemistry, flow cytometry, Western blot, ELISA, and the like.

Also provided is the use of pIRS2/MHC binding molecules, e.g., humanized, chimeric or fully human antibodies against pIRS2, antibody fragments, chimeric antibody-T cell receptors (caTCRs), chimeric antigen receptors (CARs), fusion proteins, and conjugates thereof, for diagnostic monitoring of protein levels (e.g., pIRS2 levels) in blood or tissue as part of a clinical testing procedure, e.g., to determine the efficacy of a given treatment regimen. For example, detection can be facilitated by coupling the immunoglobulin-related composition to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ³¹¹I, ³⁵S, or ³H.

This disclosure further provides a diagnostic method useful during diagnosis of pIRS2-mediated diseases such as cancers that present the RVA[pS]PTSGVK (SEQ ID NO: 19) peptide in complex with HLA-A*02. Examples of such cancers include, but are not limited to, acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, ovarian cancer, breast cancer, cervical cancer, prostate cancer, melanoma, mesothelioma, pancreatic cancer, thyroid cancer, liver cancer, hepatocellular carcinoma, or a cancer presenting the peptide of RVA[pS]PTSGVK (SEQ ID NO: 19) in complex with HLA-A*02, which involves measuring the expression level of pIRS2 in tissue or body fluid from an individual and comparing the measured expression level with a standard pIRS2 expression level in normal tissue or body fluid, whereby an increase in the expression level compared to the standard is indicative of a disorder treatable by an immunoglobulin-related composition as provided herein.

The anti-pIRS2/MHC immunoglobulin-related compositions provided herein can be used to assay pIRS2/MHC levels in a biological sample using classical immunohistological methods known to those of skill in the art (e.g., see Jalkanen, et al, J. Cell. Biol. 101:916-985 (1985); Jalkanen et al, J. Cell Biol. 105:3087-3096 (1987)). Other antibody-based methods useful for detecting pIRS2 protein or peptide expression include immunoassays, such as the enzyme linked immunosorbent assay (ELISA), immunoprecipitation, or Western blotting.

By “assaying the expression level of pIRS2/MHC” is intended qualitatively or quantitatively measuring or estimating the level of pIRS2/MHC complexes in a first biological sample either directly (e.g., by determining or estimating absolute levels) or relatively (e.g., by comparing to the disease associated levels in a second biological sample). The pIRS2/MHC complex levels in the first biological sample can be measured or estimated and compared to a standard pIRS2/MHC complex level, the standard being taken from a second biological sample obtained from an individual not having the disorder or being determined by averaging levels from a population of individuals not having the disorder. As will be appreciated in the art, once the “standard” pIRS2/MHC complex level is known, it can be used repeatedly as a standard for comparison.

Formulations of Pharmaceutical Compositions. According to the methods of the present technology, the immunoglobulin-related compositions of the present technology (e.g., antibodies such as humanized, chimeric or fully human antibodies, antibody fragments, chimeric antibody-T cell receptors (caTCRs), chimeric antigen receptors (CARs), fusion proteins, and conjugates thereof) can be incorporated into pharmaceutical compositions suitable for administration. The pharmaceutical compositions generally comprise a recombinant or substantially purified immunoglobulin-related composition and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the immunoglobulin-related compositions (See, e.g., Remington's Pharmaceutical Sciences, Mack Publishing Co., Easton, PA 18^{th} ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the composition are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the anti-pIRS2/MHC immunoglobulin-related composition, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. An anti-pIRS2/MHC immunoglobulin-related composition named in this technology can be present in unsalified or unesterified form, or in salified and/or esterified form, and the naming of such anti-pIRS2/MHC immunoglobulin-related composition is intended to include both the original (unsalified and unesterified) compound and its pharmaceutically-acceptable salts and esters. Also, certain embodiments of the present technology can be present in more than one stereoisomeric form, and the naming of such anti-pIRS2/MHC immunoglobulin-related composition is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the immunoglobulin-related compositions disclosed herein, use thereof in the compositions is contemplated. Supplementary active compounds can also be incorporated into the compositions. Other suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

A pharmaceutical composition of the present technology is formulated to be compatible with its intended route of administration. The anti-pIRS2/MHC immunoglobulin-related compositions of the present technology can be administered by parenteral, topical, intravenous, oral, subcutaneous, intraarterial, intradermal, transdermal, rectal, intracranial, intrathecal, intraperitoneal, intranasal; or intramuscular routes, or as inhalants. The anti-pIRS2/MHC immunoglobulin-related composition can optionally be administered in combination with other agents that are at least partly effective in treating various pIRS2-associated diseases (e.g., cancers).

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be desirable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating an anti-pIRS2/MHC immunoglobulin-related composition of the present technology in the required amount in an appropriate solvent with one or a combination of ingredients enumerated above, as required, followed by filtered sterilization. Generally, dispersions are prepared by incorporating the anti-pIRS2/MHC immunoglobulin-related composition into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The immunoglobulin-related compositions of the present technology can be administered in the form of a depot injection or implant preparation which can be formulated in such a manner as to permit a sustained or pulsatile release of the active ingredient.

Oral compositions generally include an inert diluent or an edible carrier. The immunoglobulin-related compositions of the present disclosure may be stabilized in some form, or protected from digestion, including but not limited to the use of D-amino acids. Oral compositions can be enclosed in gelatin capsules or compressed into tablets. For the purpose of oral therapeutic administration, the anti-pIRS2/MHC immunoglobulin-related composition can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the anti-pIRS2/MHC immunoglobulin-related composition is delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the anti-pIRS2/MHC immunoglobulin-related composition is formulated into ointments, salves, gels, or creams as generally known in the art.

The anti-pIRS2/MHC immunoglobulin-related composition can also be prepared as pharmaceutical compositions in the form of suppositories (e.g., with conventional suppository bases such as cocoa butter and other glycerides) or retention enemas for rectal delivery.

In one embodiment, the anti-pIRS2/MHC immunoglobulin-related composition is prepared with carriers that will protect the anti-pIRS2/MHC immunoglobulin-related composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

### Kits of the Present Technology

The present technology provides kits for the detection and/or treatment of pIRS2-associated diseases (e.g., cancers), comprising at least one immunoglobulin-related composition of the present technology (e.g., any antibodies (including monoclonal antibodies, polyclonal antibodies, human antibodies, humanized antibodies, chimeric antibodies, recombinant antibodies, multispecific antibodies, bispecific antibodies, etc.), antibody fragments, chimeric antibody-T cell receptors (caTCRs), chimeric antigen receptors (CARs), fusion proteins, and conjugates thereof described herein), or a functional variant (e.g., substitutional variant) thereof. Optionally, the above described components of the kits of the present technology are packed in suitable containers and labeled for diagnosis and/or treatment of pIRS2-associated diseases (e.g., cancers). The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, assay reagents, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kits are useful for detecting the presence of an immunoreactive pIRS2/MHC complex in a biological sample, e.g., any body fluid including, but not limited to, e.g., serum, plasma, lymph, cystic fluid, urine, stool, cerebrospinal fluid, ascitic fluid or blood and including biopsy samples of body tissue. For example, the kit can comprise: one or more immunoglobulin-related compositions of the present technology (e.g., antibodies such as humanized, chimeric or human antibodies, antibody fragments, chimeric antibody-T cell receptors (caTCRs), chimeric antigen receptors (CARs), fusion proteins, and conjugates thereof) capable of binding a pIRS2/MHC complex in a biological sample; means for determining the amount of the pIRS2/MHC complex in the sample; and means for comparing the amount of the immunoreactive pIRS2/MHC complex in the sample with a standard. One or more of the immunoglobulin-related compositions may be labeled. The kit components, (e.g., reagents) can be packaged in a suitable container. The kit can further comprise instructions for using the kit to detect the immunoreactive pIRS2/MHC complex.

For antibody-based kits, the kit can comprise, e.g., 1) a first immunoglobulin-related composition of the present technology, e.g. a humanized, chimeric or bispecific anti-pIRS2/MHC antibody or an antigen binding fragment thereof, attached to a solid support, which binds to a pIRS2/MHC complex; and, optionally; 2) a second, different antibody which binds to either the pIRS2/MHC complex or to the first antibody, and is conjugated to a detectable label. The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for detection of a pIRS2/MHC complex in vitro or in vivo, or for treatment of pIRS2-associated diseases (e.g., cancers) in a subject in need thereof. In certain embodiments, the use of the reagents can be according to the methods of the present technology.

### EXAMPLES

The present technology is further illustrated by the following Examples, which should not be construed as limiting in any way.

### Example 1: Materials and Methods

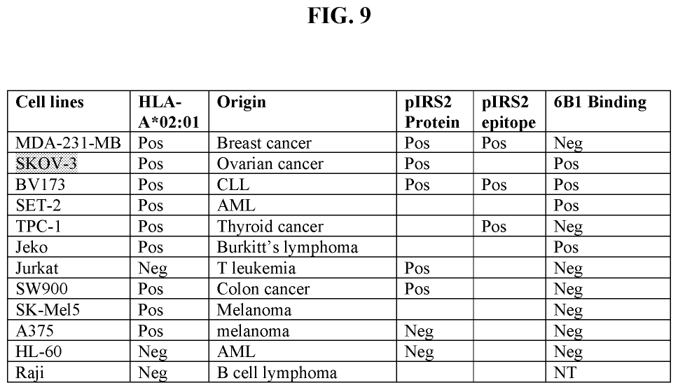

Cell samples, cell lines, and antibodies. After informed consent on Memorial Sloan-Kettering Cancer Center (MSKCC) Institutional Review Board-approved protocols, Peripheral mononuclear cells (PBMCs) from HLA-typed healthy donors were obtained by Ficoll density centrifugation. A listing of the hematopoietic and solid tumor cell lines can be found in FIG. 9.

Normal human cardiomyocytes, cardiac fibroblast and thymic fibroblasts were purchased from Science Cell Research laboratories (Carlsbad, CA). mAbs against human HLA-A2 (clone BB7.2) conjugated to fluorescein isothiocyanate (FITC) or allophycocyanin (APC), and its isotype control mouse IgG2b/FITC or APC were purchased from BD Biosciences. Goat F(ab′)2 anti-hIgG conjugated with phycoerythrin (PE) or FITC, mouse anti-human CD3 mAb, and 6×-His Tag mAb/FITC were purchased from Invitrogen. Antibodies to pSer1100-IRS2 (SC-25778), IRS2 (H-205) and GAPDH were purchased from Santa Cruz Biotechnology. APC conjugation kit-lighting link (ab201817) was purchased from Abcam and was used to label 6B1 according to manufacturer's instruction. Mouse mAb to HLA class I (W6/32) was obtained from the MSKCC Monoclonal Antibody Core Facility. Human isotype control hIgG1 antibody was purchased from Bingo Biotech (catalog number ET901). CytoTox 96 Non-radioactive cytotoxicity assay kit was obtained from Promega.

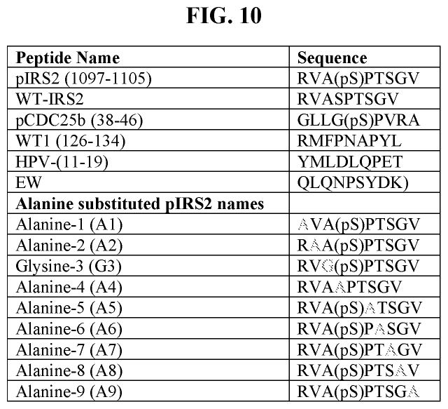

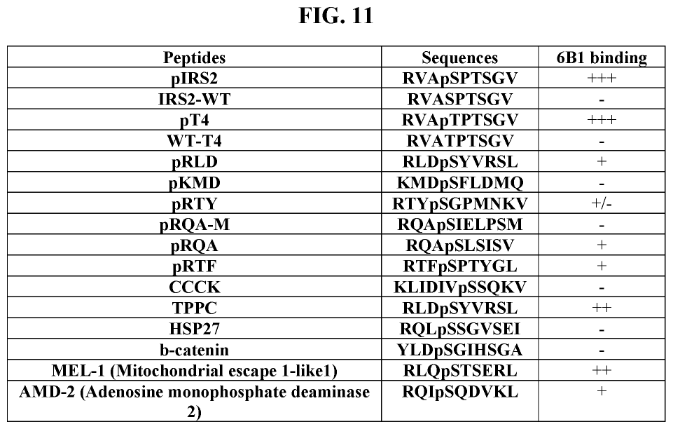

Peptides. All peptides were purchased and synthesized by Genemed Synthesis Inc (San Antonio, TX). Peptides were >95% pure (FIGS. 10-11). The peptides were dissolved in dimethyl sulfoxide and diluted in saline at 5 mg/ml and frozen at −80° C.

Flow cytometry analysis. For cell surface staining, cells were incubated with appropriate mAbs for 30 min on ice, washed, and incubated with secondary antibody reagents when necessary. Live cells were gated based on FSC and SSC and then unstained cells are used as negative control. All groups have the same gate and then each group was overlaid as histograms for comparison. Unstained cells and control mAb staining were shown as controls. Flow cytometry data were collected on a LSR Fortessa™ (BD Biosciences) and analyzed with FlowJoV10.6.1 software.

EBV-specific Tcell expansion. T-cells were enriched from PBMCs by depletion of monocytes by adhesion. Non-adhering cells were stimulated with irradiated autologous EBV-transformed B cells (EBV-BLCLs) generated by transformation with the B95.8 strain of EBV at a 20:1 responder/stimulator (R/S) ratio and cultured in RPM11640 medium, containing 10% HS. Beginning on day 7, interleukin (IL)-2 at 20 to 80 units/ml (Collaborative Biomedical Products, Bedford, MA) and IL-15 at 10 ng/ml (NCI) were added to the T-cell cultures every 2-3 days and were re-stimulated weekly with the same EBV-BLCLs at a 4:1 R/S ratio.

### Validation of the Targets

1. T cell response. Immunogenicity of the pIRS2 peptide was validated using T cell response as surrogate. PBMCs from HLA-A*02:01 healthy donors were obtained by Ficoll density centrifugation. CD14+ monocytes were isolated and used for antigen presenting cells (APCs) and for dendritic cell (DC) generation to stimulate T cells in vitro. CD3+ T cells were isolated by negative immunomagnetic cell separation using a pan T cell isolation kit. T cells were stimulated with autologous APCs for three to four rounds and peptide-specific response was determined by IFN-gamma ELISpot, as described previously (May R, Dao T, Pinilla-Ibarz J, Korontsvit T, Zakhaleva V, Zhang R H, Maslak P, Scheinberg D A (2007). Clin Cancer Res 13:4547-4555, Pinilla-Ibarz J, May R J, Korontsvit T, Gomez M, Kappel B, Zakhaleva V, Zhang R H & Scheinberg D A. Leukemia 2006; 20 (11): 2025-2033).

2. Mass spectrometry (Mass spec). pIRS2 epitope presented by HLA-A2 molecule was detected by mass spectrometry as described before (Klatt M G, et al., JCI Insight. 2020; 5(19)) in thyroid cancer TPC-1, breast cancer cell line MDA-MB-231, Burkitt lymphoma cell lines Jeko and NCEB1 and ALL cell line BV173, without specific phosphopeptide-enrichment, suggesting a relatively high density of the epitope on the cell surface. Suspension cells were harvested through direct resuspension, and adherent cell lines were harvested after incubating 15 minutes with CellStripper solution (Corning, catalog 25056CI). Harvested cells were pelleted and washed 3 times in ice-cold sterile PBS (Media Preparation Core, Memorial Sloan Kettering Cancer Center). 20 million cells were used per experiment. Cells were lysed in 7.5 mL of 1% CHAPS (MilliporeSigma, catalog C3023) dissolved in PBS, supplemented with protease inhibitors (cOmplete, Roche, catalog 11836145001). Cell lysis was performed for 1 hour at 4° C., lysates were spun down for 1 hour with 20,000 g at 4° C., and supernatant fluids were isolated.

Forty mg of cyanogen bromide-activated-Sepharose 4B (MilliporeSigma, catalog C9142) was activated with 1 mM hydrochloric acid (MilliporeSigma, catalog 320331) for 30 minutes. Subsequently, 0.5 mg of W6/32 antibody (Bio X Cell, catalog BE0079) were coupled to Sepharose in the presence of binding buffer (150 mM sodium chloride, 50 mM sodium bicarbonate, pH 8.3; sodium chloride: MilliporeSigma, catalog S9888; sodium bicarbonate: MilliporeSigma, catalog S6014) for at least 2 hours at room temperature. Sepharose was blocked for 1 hour with glycine (MilliporeSigma, catalog 410225) and washed 3 times with PBS.

Supernatants of cell lysates were run over the different types of columns through peristaltic pumps (Pharmacia Biotech, Model P-1) with 1 mL/min flow rate overnight in a cold room. Affinity columns were washed with PBS for 30 minutes and water for 30 minutes, then run dry, and HLA complexes were subsequently eluted 5 times with 200 μL 1% TFA (MilliporeSigma, catalog 02031).

For separation of HLA ligands from the HLA complexes, C18 columns (Sep-Pak C18 1 cc Vac Cartridge, 50 mg sorbent per cartridge, 37-55 m particle size, Waters, catalog WAT054955) were preconditioned with 80% ACN (MilliporeSigma, catalog 34998) in 0.1% TFA and equilibrated with 2 washes of 0.1% TFA. Samples were loaded, washed again with 0.1% TFA, and eluted in 400 μL of 30%, 40%, or 50% ACN in 0.1% TFA. Sample volume was reduced by vacuum centrifugation for mass spectrometry analysis.

Solid-phase extractions. In-house C18 mini-columns were prepared as follows: for solid-phase extraction of 1 sample, 2 small disks of C18 material (1 mm in diameter) were punched out from CDS Empore C18 disks (Thermo Fisher Scientific, catalog 13-110-018) and transferred to the bottom of a 200 μL Axygen pipette tip (Thermo Fisher Scientific, catalog 12639535). Columns were washed once with 100 μL 80% ACN/0.1% TFA and equilibrated 3 times with 100 μL 1% TFA. All fluids were run through the column by centrifugation in mini tabletop centrifuges, and eluates were collected in Eppendorf tubes. Then, dried samples were resuspended in 100 μL 1% TFA and loaded onto the columns, washed twice with 100 μL 1% TFA, run dry, and eluted with 50 μL 80% ACN/0.1% TFA. Sample volume was reduced by vacuum centrifugation.

Liquid chromatography-tandem mass spectrometry analysis of HLA ligands. Samples were analyzed by high-resolution/high-accuracy liquid chromatography-tandem mass spectrometry (LC-MS/MS) (Lumos Fusion, Thermo Fisher Scientific). Peptides were separated using direct loading onto a packed-in-emitter C18 column (75 m ID/12 cm, 3 m particles, Nikkyo Technos Co., Ltd.). The gradient was delivered at 300 nL/min increasing linearly from 2% buffer B (0.1% formic acid in 80% ACN)/98% buffer A (0.1% formic acid) to 30% buffer B/70% buffer A, over 70 minutes. MS and MS/MS were operated at resolutions of 60,000 and 30,000, respectively. Only charge states 1, 2, and 3 were allowed. The isolation window was chosen as 1.6 Thomsons, and collision energy was set at 30%. For MS/MS, maximum injection time was 100 ms with an automatic gain control of 50,000.

MS data processing. MS data were processed using Byonic software (version 2.7.84, Protein Metrics) through a custom-built computer server equipped with 4 Intel Xeon E5-4620 8-core CPUs operating at 2.2 GHz and 512 GB physical memory (Exxact Corporation). Mass accuracy for MS1 was set to 6 ppm and to 20 ppm for MS2. Digestion specificity was defined as unspecific, and only precursors with charges 1, 2, and 3 and up to 2 kDa were allowed. Protein FDR was disabled to allow complete assessment of potential peptide identifications. Oxidization of methionine; phosphorylation of serine, threonine, and tyrosine; as well as N-terminal acetylation were set as variable modifications for all samples. Samples were searched against UniProt Human Reviewed database (20,349 entries, www.uniprot.org, downloaded June 2017) with common contaminants added. Peptides were selected with a minimal log probability value of 2, indicating P values for peptide spectrum matches of less than 0.01 and duplicates removed.

3. Western blot analysis. Cell lysates were generated using RIPA buffer with protease and phosphatase inhibitor and quantified using the DC protein assay (Bio-Rad). Protein (20-30 μg) was loaded and separated on 4% to 15% gradient SDS/PAGE gels (Bio-Rad). Proteins were transferred to Nitrocellulose membranes (Thermo Fisher Scientific, 88018), which were blocked for 2 hour with 5% milk at room temperature. Immunoblotting was using pSer1100-IRS2-specific (Thermo Fisher Scientific; PA5-106094) and with anti-IRS-2 (Abcam; EPR904(2)). Both antibodies were probed at the manufacturer's recommended dilution overnight at 4° C. before using a secondary antibody directly conjugated to HRP for imaging. Blots were re-probed with an anti-GAPDH-HRP direct conjugated Ab (Cell Signaling Technology, 3683) as a loading control.

Production of pIRS2 HLA-A2 Peptide Complexes

The method used follows directly the original protocol established by David Garboczi (Garboczi, et al., J. Immunol. 1996; 157:5403-5410; Garboczi, et al., Nature 1996; 384:134-141). Briefly, large amounts of soluble MHC class I/peptide complexes were generated by overexpression of HLA-A2 heavy chain (HC) and beta2 microglobulin (β2m) as recombinant proteins in E. Coli and subsequent in vitro refolding and assembly in the presence of high concentrations of pIRS2 peptide. Unphosphorylated IRS2 as well as WT1 peptides were used to generate HLA-A2 complexes to be used as counter selection controls. To obtain soluble MHC/peptide complexes, the HC sequence was mutagenized to remove the cytosolic and transmembrane regions. In order to specifically biotinylate refolded, monomeric MHC/peptide complexes, the HC was expressed as a fusion protein containing a specific biotinylation site at the C-terminus (Altman, J. D., et al., Science 274:94-96 (1996); Busch, D. H., et al., Immunity 8:353-362(1998)). These short sequences are sufficient for site-specific, enzymatic in vitro biotinylation of a single lysine residue within this sequence using the biotin protein ligase BirA (Schatz, P. J. Biotechnology 11:1138-1143 (1993)). Size exclusion chromatography (SEC) was used to separate stable complexes from free β2m and biotin in case protein was in vitro biotinylated.

### Screening of Phage Library and Engineering of Full Length Human IgG1

A proprietary naïve, semi-synthetic scFv phage display library (Valadon P, et al., MAbs.11(3):516-531 (2019)) was screened for human antibodies that bind the pIRS2/HLA-A2 complex by using standard solution phase phage display panning techniques. Briefly, the protein complex was incubated with the phage library and captured using streptavidin coated magnetic beads. Subsequent bead capture, washing, elution and phage amplification steps were performed for each round of bio-panning. Three rounds of panning were completed using amplified pIRS2/HLA-A2 complex binder-enriched phage pools from the previous round of panning as input for subsequent rounds. Each round of panning included a negative selection step against HLA-A2 protein complexed with unphosphorylated IRS2 peptide, IRS2/HLA-A2complex.

To identify scFv fragments that showed high specificity for to the pIRS2/HLA-A2 complex, single clones from the third round of panning were analyzed for binding to pIRS2/HLA-A2 protein complex, and BSA (as a non-specific control) by enzyme-linked immunosorbent assay (ELISA) using an anti-M13 phage monoclonal antibody. Monoclonal phage supernatants that showed pIRS2/HLA-A2 complex-specific binding were selected for antibody sequencing and screened for binding to T2 cells pulsed with pIRS2, IRS2 or irrelevant MHC class I phospho-peptides. Variable regions of selected hits were formatted as full-length human IgG1, followed by expression of the antibodies in mammalian cells and purification.

### Biolayer Interferometry (BLI)

The OctetRed™ system (ForteBio, Pall LLC) was used to determine the binding properties of mAb 6B1. Biotinylated pIRS2/HLA-A2 was captured with SA (streptavidin) biosensors and the binding was monitored in a 2-fold dilution series of 6B1 starting at 30 nM. The experiment was carried out using kinetic buffer (PBS pH 7.4, 0.01% BSA, 0.002% Tween-20). Response curves were globally fitted to a 1:1 Langmuir binding model to determine values for the k-on (association rate constant), k-off (dissociation rate constant), and KD (equilibrium dissociation constant).

Characterization of the full-length hIgG1 for the pIRS2A2 complex. Initially, the specificities of the fully human IgG1mAbs for the pIRS2/A2 complex were determined by staining T2 cells pulsed with or without pIRS2, IRS2 or other HLA-A2-binding irrelevant peptides, followed by secondary goat F(ab′)₂ anti-hIgG mAb conjugated to PE or FITC. The fluorescence intensity was measured by flow cytometry. Direct staining was also performed by conjugating the mAbs with fluorophore APC. The same methods were used to determine the binding of the mAb to fresh tumor cells and cell lines.

Antibody-dependent cellular cytotoxicity (ADCC). Target cells used for ADCC were T2 cells pulsed with or without pIRS2, IRS2- or irrelevant HLA-A2 binding peptides, and cancer cell lines without peptide pulsing. pIRS2 mAbs or its isotype control human IgG1 at various concentrations was incubated with target cells and fresh PBMCs (as effectors) at different effector/target ratios for 16 hours. The supernatant was harvested, and the cytotoxicity was measured by LDH release assay with CytoTox 96 non-radioreactive kit from Promega following their instruction. Percent cytotoxicity was calculated using following formula:

$$\% Cytotoxicity = Experimental - Effector spontaneous - Target spontaneous / Target maximum - Target spontaneous \times 100.$$

Cytotoxicity was also measured by standard 5-hour ⁵¹Cr release assay. ADCC assays were not statistically comparable between experiments because the sources of human cells changed; in individual experiments, means of triplicate results were normalized. In the case of using BisAbs of 6B1, EBV-specific T cells were used as effector cells as described previously (Dao T, et al., Nat. Bio. Tech. 2015; doi:10.1038/nbt.3349). For testing cytotoxicity against AML PDXs, the target cells were labeled with CFSE and co-incubated with activated T cells (CD3/CD28 beads) in the presence of 6B1 (1+1) mAb or its isotype control (10 μg/ml) at E:T ratio 6:1 overnight. The cells were harvested, washed and stained with mAbs for CD33 and other AML markers and zombie (dead cells) and run on flow cytometry. The percentage reduction of CFSE positive cells or CFSE and Zombie double positive cells over CFSE positive cells were determined as % lysis of targets.

### Bispecific mAb (BisAb) Engineering.

All T-cell dependent BisAb designs were based on a human IgG1 scaffold, possessing the L234A/L235A (LALA) mutations to prevent antibody effector function. The L2K single chain variable fragment (scFv) in vH-vL orientation linked via an 18-amino acid linker (GEGTSTGSGGSGGSGGAD; SEQ ID NO: 43) served as the anti-CD3 moiety for T-cell recruitment. BisAb C2+2 was constructed by fusing L2K scFv sequences to the C-terminus of the heavy chain of a 6B1 IgG1, separated by a 15-amino acid linker ((GGGGS)₃; SEQ ID NO: 44). BisAb h2+2 was generated by flanking the L2K scFv with 10-amino acid linkers ((GGGGS)₂; SEQ ID NO: 45) and inserting into the upper hinge region between C220 and D221. For the asymmetric T-cell dependent BisAbs the Fc region was mutated to create a “knob” (T366W) or “hole” (T366S, L368A and Y407V) for heteromeric assembly of the respective heavy chains. BisAb 1+1 consists of a 6B1-IgG1 “hole” and a L2K scFv fused to the IgG1 Fc “knob” via a 15-amino acid linker ((GGGGS)₃; SEQ ID NO: 44) to the upper hinge. Similarly, BisAb h2+1 was constructed by combining a 6B1-IgG1 “hole”, with a 6B1-IgG1 “knob” in which the L2K scFv is inserted into the hinge region via two flanking 10-amino acid linkers ((GGGGS)₂; SEQ ID NO: 45).

The production of all antibodies was carried out at GenScript®. Briefly, antibody sequences were generated by gene synthesis and cloned into cytomegalovirus promoter-driven expression vectors. All proteins were expressed by transient co-transfection in HD 293F cells, purified by affinity chromatography, followed by size exclusion chromatography to obtain the desired purity. The purified antibodies were analyzed by SDS-PAGE, Western blot, and HPLC analysis to determine the molecular weight and purity.

### Modeling TCRm Binding to Phosphopeptide-HLA-A2 Complexes

Models of vH and vL fragments of 6B1 were generated from their respective protein sequences by grafting CDRs onto their canonical framework structures using the LYRA webserver (Klausen, M. S., et al., Nucleic Acids Res. 2015; 43, W349-W355W355). The resulting 6B1 model was docked to the previously published crystal structure of the pIRS2/HLA-A2 complex (PDB ID: 3FQX) (30) using the ClusPro 2.0 webserver in antibody mode (Desta, I. T., et al., Structure 28, 1071-1081.e3 (2020); Brenke, R. et al., Bioinformatics 28, 2608-2614 (2012)). The top scoring complexes based on their clustering properties were then manually inspected for models in which CDR loops were in contact with the phosphopeptide-MHC surface. The resulting models were used as templates for initial poses of 6B1 in complex with pIRS2/HLA-A2 and for other phosphopeptides by mutating the bound phosphopeptide to pT4, pKMD, and pRTF in UCSF Chimera using the Dunbrack and SwissSideChain rotamer libraries (Pettersen, E. F. et al., J. Comput. Chem. 25, 1605-1612 (2004); Shapovalov, M. V. & Dunbrack, R. L., Structure 19, 844-858 (2011); Gfeller, D., et al., Nucleic Acids Res. 41, D327-D332 (2013)). Initial poses of 6B1 in complex with pIRS2/HLA-A2 and other phosphopeptides in complex with HLA-A2 were submitted to the FlexPepDock web server to determine the most likely phosphopeptide conformation for each phosphopeptide in complex with HLA-A2 and 6B1 (London, N., et al., Nucleic Acids Res. 39, W249-W253 (2011); Raveh, B., et al., Proteins 78, 2029-2040 (2010)). For each distinct phosphopeptide, the top 10 lowest energy poses, as determined by their Rosetta energy score, were selected among 300 high resolution models. To compare the energetic favorability of 6B1 binding to each distinct phosphopeptide/HLA-A2 complex, the binding interface energy of the top 10 scoring models for each distinct complex was computed using the InterfaceAnalyzer application (Lewis, S. M. & Kuhlman, B. A., PLoS One 6, e20872 (2011)) implemented in Rosetta 3 (Leaver-Fay A., et al., Methods Enzymol. 2011; 487:545-574). All models were visualized in UCSF Chimera with molecular surfaces computed by the MSMS package (Sanner, M. F., et al., Biopolymers 38, 305-320 (1996)).

### Example 2: Validation of Target pIRS2 in the Context of HLA-A*02:01

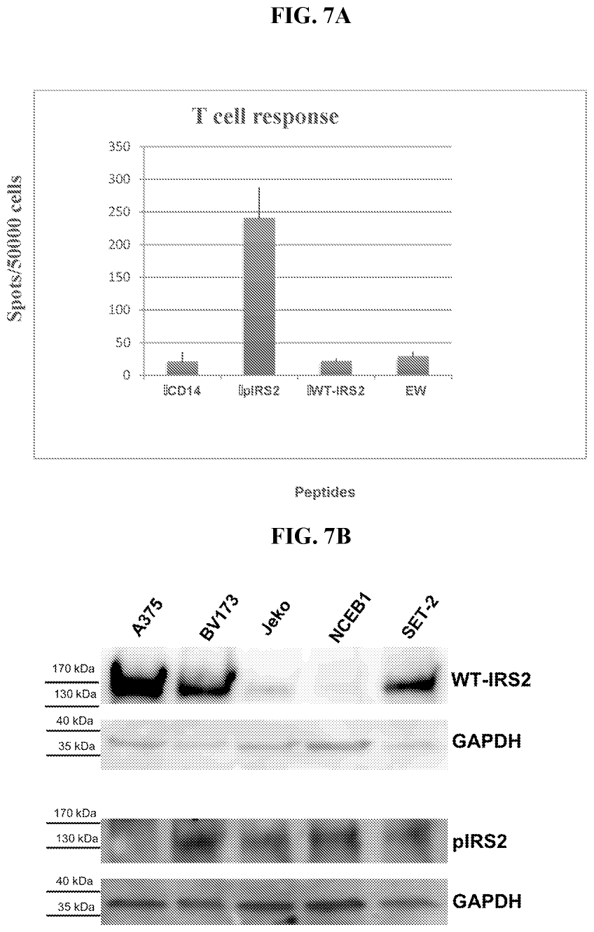

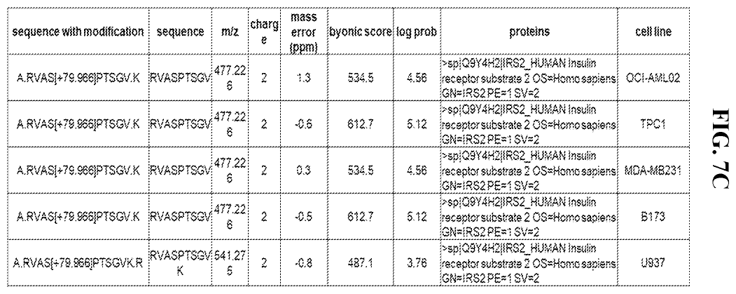

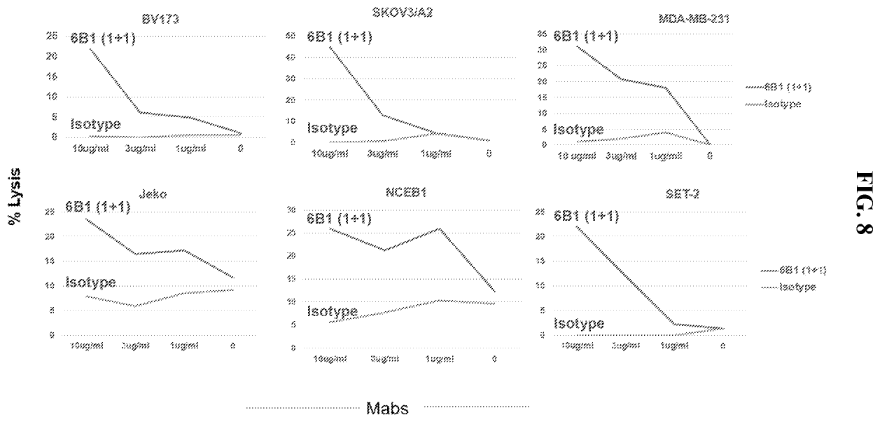

Validation of the target on cancer cells was assessed by confirming T cell reactivity against pIRS2 and presentation of the epitope by mass spectrometry. To test if pIRS2 induces a T cell response that has no cross-reactivity to the unphosphorylated peptide (IRS2), purified CD3 T cells from HLA-A*02:01+ donors were stimulated with pIRS2 and the peptide-specific response was measured by IFN-gamma ELISpot assay. The pIRS2 elicited peptide-specific T cell responses that did not cross react with the cognate native peptide (FIG. 7A). These data confirmed that T cells directed against pIRS2/HLA epitopes could be specific for tumor cells undergoing dysregulated phosphorylation that discriminate against non-phosphorylated IRS2 in normal cells.

The presentation of the pIRS2 on different cancer cell lines was tested by HLA ligand isolation and subsequent mass spectrometry. Two HLA-A02 positive hematopoietic (BV173, an ALL; OCI-AML02, an AML) and two HLA-A02 positive, non-hematopoietic cell lines (MDA-MB231, a breast cancer; TPC1, a thyroid cancer) were chosen, and presentation of the pIRS2 derived HLA ligand was confirmed in all four cell lines. Additionally, consistent with published literature, a length variant of the RVA(pS)PTSGV peptide could be detected in another HLA context: RVA(pS)PTSGVK on HLA-A*03 of U937 cells (FIG. 7C).

To additionally validate these results, the native IRS2 protein and its phosphoprotein in a panel of tumor cells lines was also evaluated by using an antibody specific for the phosphorylated IRS2 protein (against pSer1100), as well as an antibody that recognizes total IRS2 protein. The A375 cell line expressed unphosphorylated IRS2 but not pIRS2. BV173, Jeko, NCEB1 and SET-2 expressed both IRS2 and pIRS2 protein (FIG. 7B).

### Example 3: Selection of scFv Specific for pIRS2/A2 Complex and Engineering of Full-Length Human mAb

Single phage clones selective for the pIRS2/A2 complex were first counter screened against WT1-RMF/A2 and native IRS2/A2 monomers (WT-IRS2) to remove clones that bind to HLA-A2 and arginine at the first position (R-1) of the peptide in the complex, and any clones that bind to native IRS2/A2 complex. Positively selected clones were then screened against pIRS2/A2 monomers. Twenty-five phage clones specific for pIRS2/A2 were confirmed by ELISA coated with biotinylated pIRS2/A2 complex. Clones that had unique DNA coding sequences were characterized in secondary screens by binding to live cells using a transporter associated with antigen processing (TAP)-deficient, human HLA-A0201+ cell line (T2) alone, pulsed with pIRS2 peptide, WT-IRS2, or other control peptides. Six of 25 clones screened showed specific binding to T2 cells pulsed with pIRS2 peptide, but not to T2, IRS2 or other control peptides. The variable regions of these six clones were formatted as full length human IgG1 antibodies for further characterization.

### Example 4: Specificity of the 6B1 Human I-GI mAb

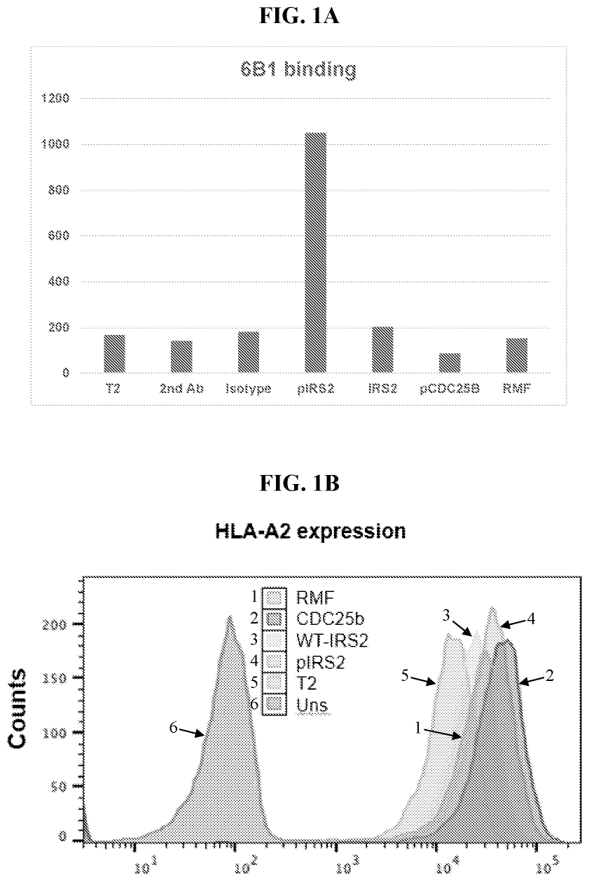

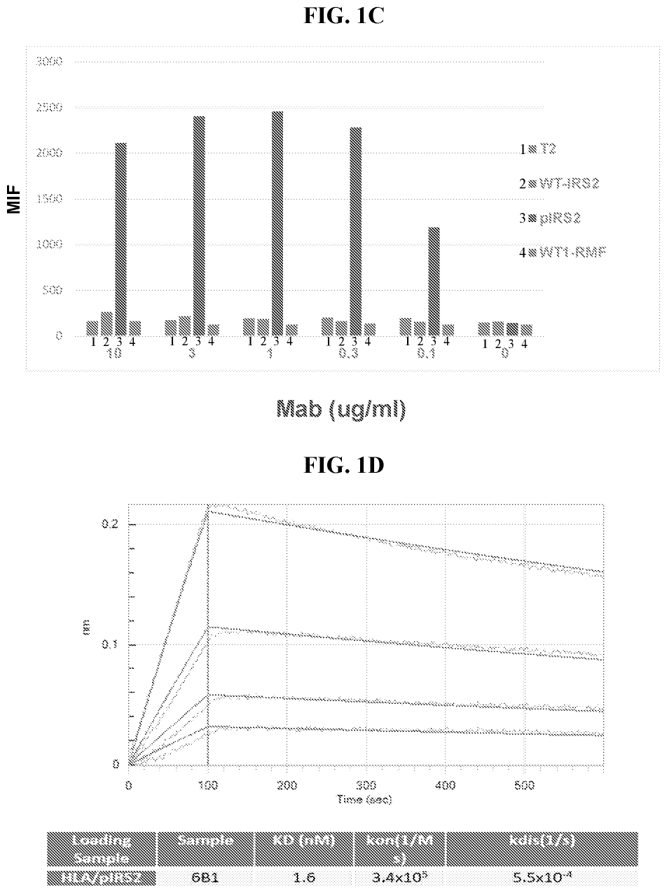

To characterize the specificity of the full length human IgG1 mAbs, T2 cells, pulsed with or without pIRS2, WT-IRS2 or other control peptides, initially were used to determine the binding specificity. One hIgG1, named 6B1, was selected out of total six mAbs based on its specificity. Mab 6B1 only bound to pIRS2-pulsed T2 cells, but not WT-IRS2 peptide and other controls pCDC25b or RMF peptides (FIG. 1A). Negative binding to T2 cells pulsed with WT-IRS2, pCDC25b or RMF was not due to poor binding of the peptides to HLA-A2, because all the peptides stabilized HLA-A2 expression (FIG. 1B) Binding avidity of 6B1 was further analyzed by titrating the mAb to pIRS2 peptide. No significant changes were seen in the binding to T2 pulsed with pIRS2 peptide, down to a mAb concentration of 0.1p g/ml, nor was significant binding observed with T2 cells alone, pulsed with WT-IRS2 or WT1-RMF peptide (FIG. 1C). Affinity of 6B1 to the pIRS2/HLA-A2 complex in solution was determined to be 1.6 nM by using Biolayer interferometry kinetics assay (FIG. 1D).

The phage screening strategy was designed to select clones that recognized both specific amino acids and the phosphate moiety on the serine residue, which would mimic TCR recognition. To analyze which amino acids of the pTRS2 peptide were important for recognition by the 6B1 mAb, the binding of the mAb to T2 cells pulsed with analog pIRS2 peptides was analyzed. pIRS2 peptide was substituted with alanine at position 1, 2, 4, 5, 6, 7, 8 and 9, or with glycine at position 3 (peptides are named A1 to A9, respectively, except for G3) (FIG. 10). The analog peptides were loaded onto T2 cells and tested for 6B1 mAb binding and for HLA-A02 expression. Alanine substitution at positions 1 and 4 strongly reduced the binding of the 6B1 mAb (FIG. 2A). HLA-A2 expression was partially reduced only with substitution at position 4, but not significantly impaired by alanine substitution at any other positions (FIG. 2B), suggesting that positions 1 and 4 were the most important residues for the mAb recognition and further demonstrating that the 6B1 mAb recognized the phosphate moiety on serine at position 4. These data supported the previous studies in TCRs specific for the phosphorylated peptides bound to HLAs by which phosphorylation-generated neoepitopes discriminate their native sequences (13, 15). The mAb 6B1 showed a similar recognition pattern as the TCR for the pIRS2/HLA-A*02:01, and the native sequence was not recognized.

To further demonstrate that the phosphorylated side chain on position 4 of the pIRS2/HLA-A2 complex was important for 6B1 mAb recognition, the serine at position 4 of the pIRS2 peptide was substituted with threonine (FIG. 11) and tested for 6B1 recognition by pulsing onto T2 cells. Mab 6B1 did not bind to unphosphorylated threonine (WT-T4). However, 6B1 bound to the pT peptide (pT-4) at a similar level as to pIRS2. These results further confirmed that 6B1 recognized the phosphate moiety on position 4 of pIRS2/HLA-A2 complex, regardless of either it was on serine or threonine (FIG. 2C). T2 stabilization assays showed that all the peptides bound to HLA-A2. Peptide pT-4 showed stronger binding to HLA-A2 than its native WT-T4 (FIG. 2D), due to the unique feature of phosphopeptides bound to HLA-A2 molecules.

To test if 6B1 was able to recognize the naturally processed pIRS2 epitope presented by HLA-A*02:01 molecules, tumor cell lines that are HLA-A*02:01 and pIRS2 positive (FIGS. 7B-7C) were tested for the binding of 6B1 by flow cytometric analysis. 6B1 was able to bind tumor cell lines CVL/ALL BV173, AML SET-2, ovarian cancer SKOV-3 and mantle cell lymphoma Jeko (FIGS. 2E-2F, 2H, and 2I). Although the pIRS2 epitope was detected by mass spectrometry and western blot in two other cell lines, MBA-MD-231 (breast cancer) and TPC-1 (thyroid cancer cell line), no binding of 6B1 was detected by flow cytometry (Not shown). It is possible that the epitope density is too low on these solid tumor cells to be detected by flow cytometric analysis. In addition, 6B1 did not bind to the HLA-A*02:01-negative cell line Jurkat (FIG. 2K). The results demonstrated that the mAb 6B1 was able to detect naturally presented pIRS2 epitope on tumor cells.

Interferon gamma treatment further enhanced the binding of the 6B1 to these tumor cell lines (FIG. 14).

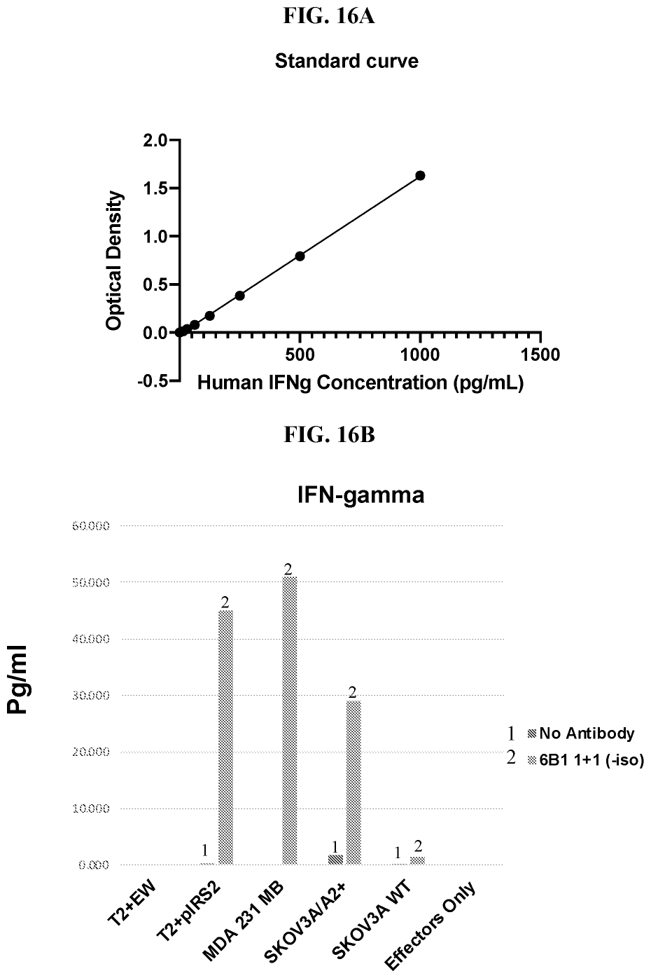

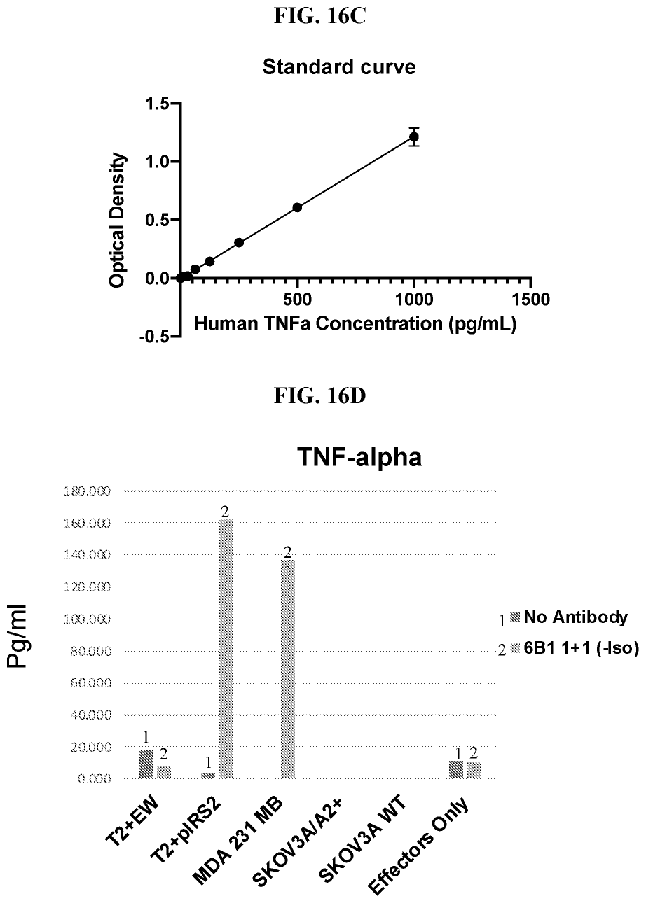

IFN-gamma and TNF-alpha secretion were evaluated via ELISA in the supernatants of the co-culture of effectors and target cells overnight. Culture conditions were similar to those in the cytotoxicity tests. Both cytokines showed modest secretion, as shown in FIGS. 16A-16D. Tumor cells were also treated with IFN-gamma and the expression of the HLA level and 6B1 binding were also tested. Modest HLA-A2 upregulation was observed that did not change 6B1 mAb recognition.

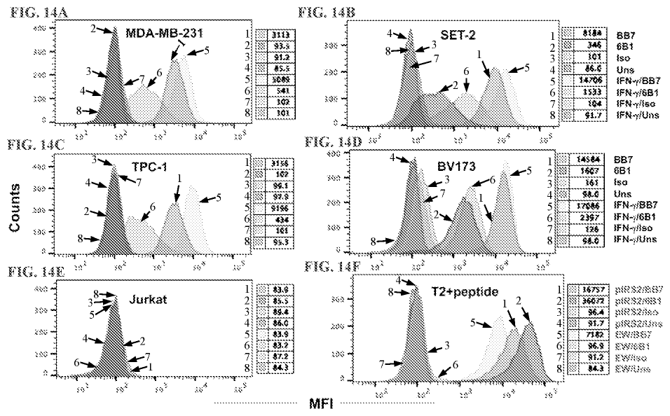

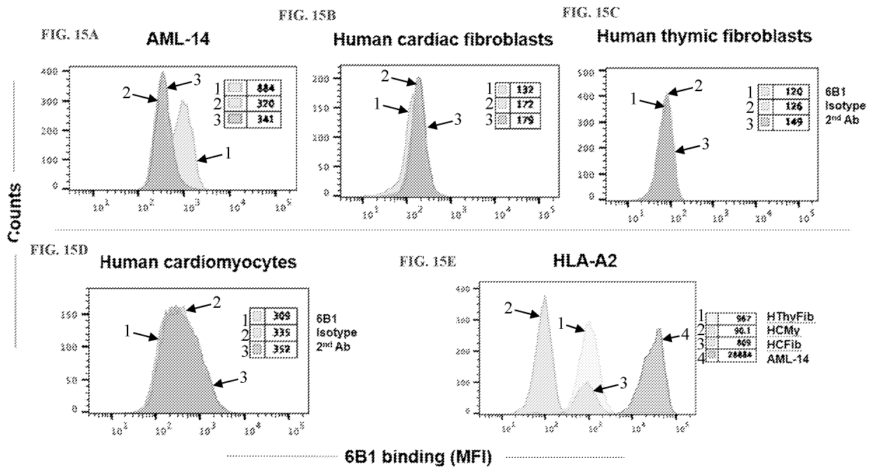

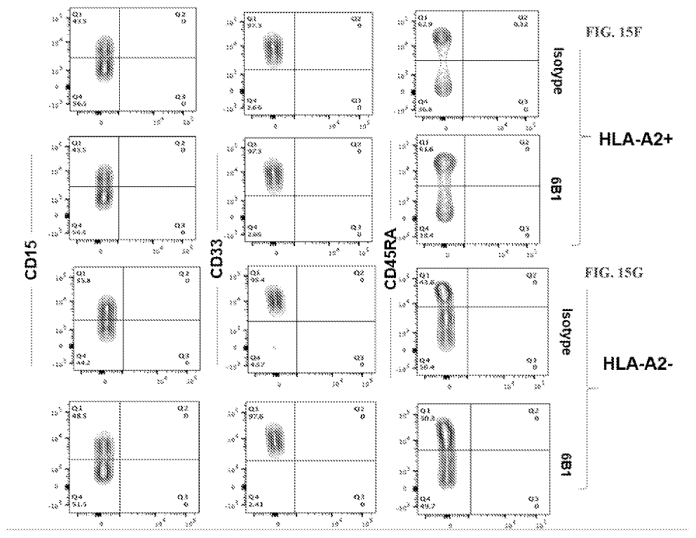

To test if 6B1 recognizes the epitope on human normal cells, normal human cardiac fibroblasts and thymic fibroblasts (both HLA-A2 positive), and human cardiomyocytes (HLA-A2 negative) were tested for the binding by 6B1 mAb (FIGS. 15B-15E). Compared to the positive control cell AML-14, that strongly bound 6B1 (FIG. 15A), all these three cells were negative for 6B1 binding. The data demonstrate that the 6B1 does not recognize these normal cells, which most likely did not phosphorylate the peptide sequence. The possibility of the mAb recognizing any normal hematopoietic cells was further evaluated by staining whole blood with the 6B1 mAb and cell lineage markers CD15 (neutrophils), CD33 (monocytes/macrophages) and CD45 (lymphocytes). 6B1 mAb did not bind to these cell populations in either HLA-A*02:01 positive (FIG. 15F) or negative donors (FIG. 15G).

Thus, 6B1 did not recognize several normal human primary cells tested, that included human cardiac and thymic fibroblasts and human cardiomyocytes. Further, 6B1 did not bind to healthy donor hematopoietic cells that included neutrophils, monocytes/macrophages and lymphocytes. This is consistent with previous reports that this phosphorylation site of IRS2 was not detected in normal hematopoietic cells.

These results demonstrate that the compositions of the present technology are useful in methods for treating pIRS2-associated diseases (e.g., cancers) in a subject in need thereof.

### Example 5: Antibody-Dependent Cellular Cytotoxicity (ADCC) of mAb 6B1

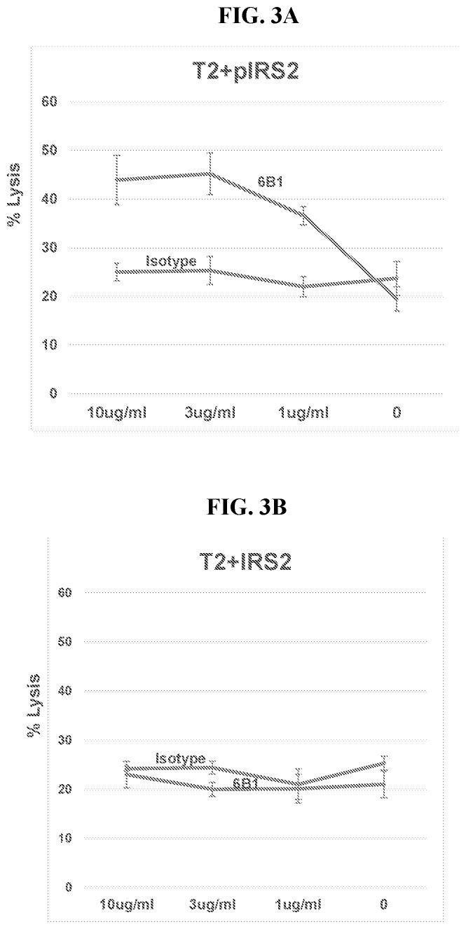

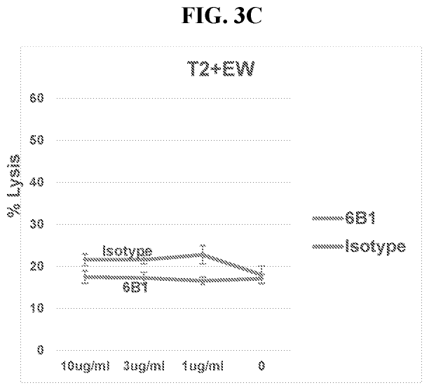

Similar to its binding specificity, 6B1 was able to mediate specific ADCC against T2 cells pulsed with pIRS2 peptide but not wild type IRS2 or irrelevant peptides (FIGS. 3A-3C). This demonstrated the functional cytolytic specificity of 6B1 for the pIRS2/HLA-A2 complex. However, ADCC activity against tumor cell lines without peptide pulsing was very limited, which might be a result of the low density of the target complexes on the cell surface. To enhance its effector functions, the 6B1 mAb was engineered into more potent formats, such as BisAbs engaging T cells via CD3.

These results demonstrate that the compositions of the present technology are useful in methods for treating pIRS2-associated diseases (e.g., cancers) in a subject in need thereof.

### Example 6: Specificity and Cytotoxic Function of 6B1-BisAbs

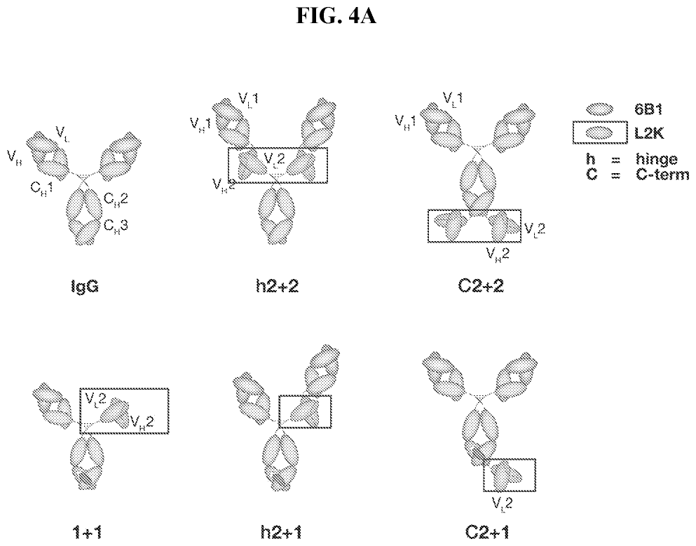

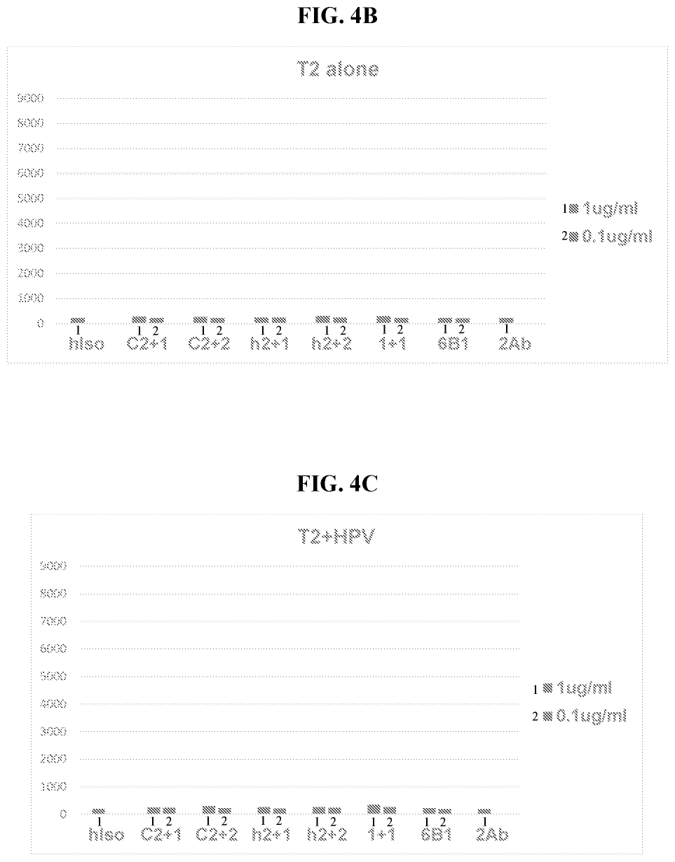

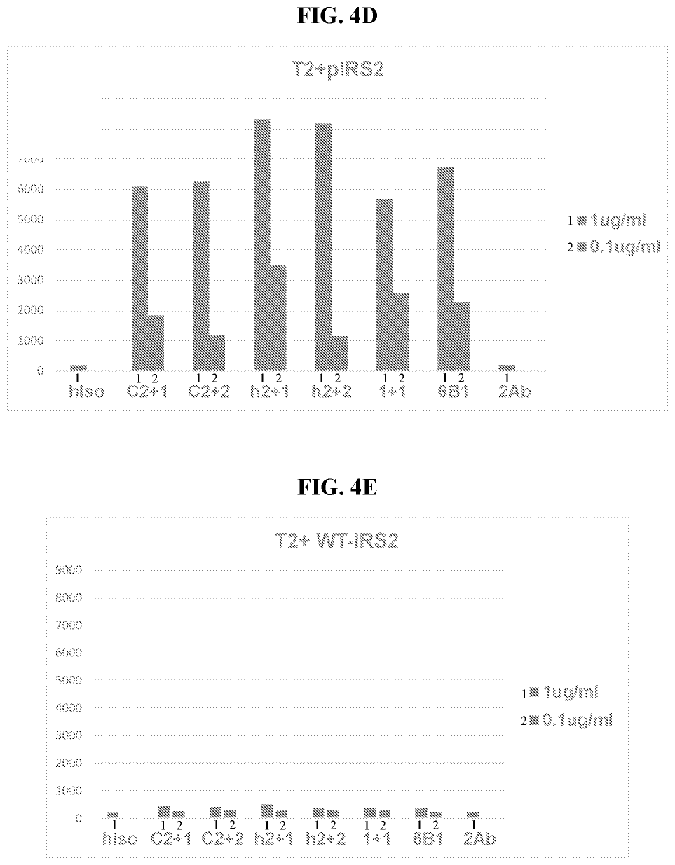

To overcome the short half-life of the small BiTE molecule, which consists of two linked scFv fragments, a series of BisAbs in human IgG formats were designed, thus preserving the favorable pharmacokinetic profile of full-length antibodies (FIG. 4A). L2K (anti-CD3 mAb) was linked to the 6B1 IgG1 format at various positions, in either monovalent (1+1, H2+1, or C2+1) or fully bivalent forms (H2+2, or C2+2).

All the engineered 6B1-BisAbs maintained their antigenic specificity for the pIRS2/HLA-A2 complex when tested for binding to T2 cells, pulsed with or without pIRS2, WT-IRS2 or HLA-A2-binding HPV E7-derived peptide (FIGS. 4B-4E). All five bispecific mAb constructs showed binding to T2 cells pulsed with pIRS2 peptide (FIG. 4D) to various degrees. No binding to T2 cells alone (FIG. 4B), pulsed with WT-IRS2 (FIG. 4E) or HPV peptide (FIG. 4C) was observed. These results showed that all BisAbs have maintained their specificity for the target antigen.

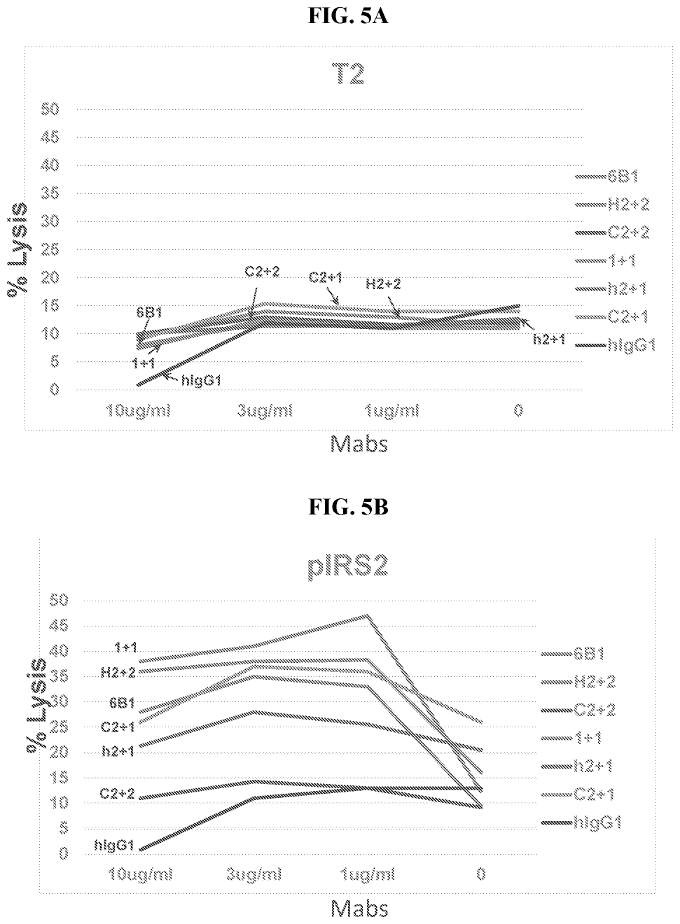

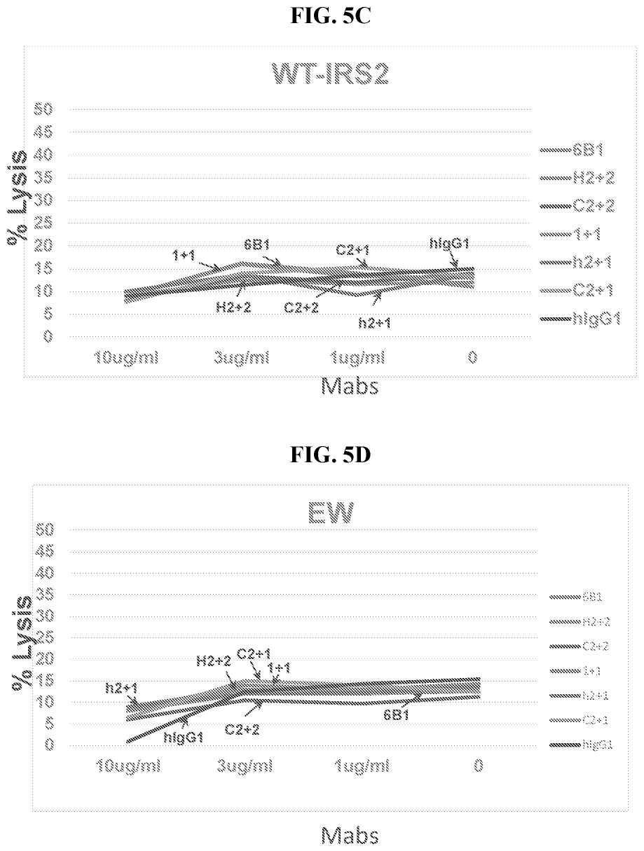

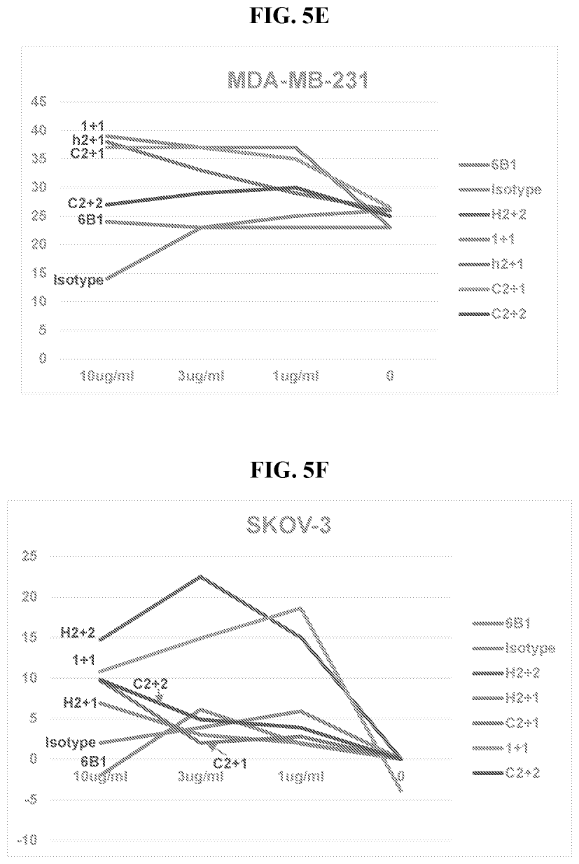

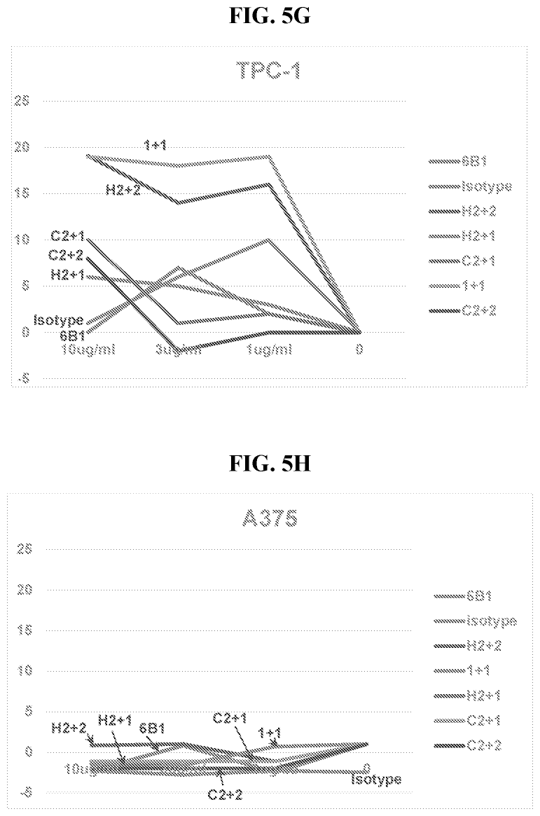

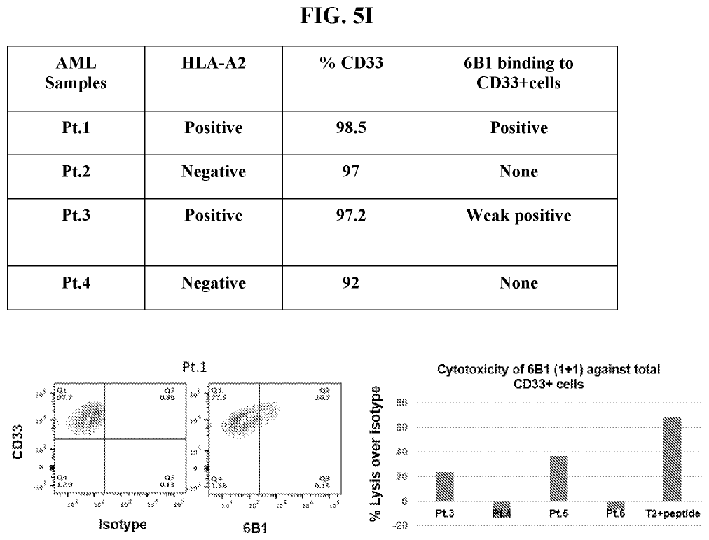

To determine if the 6B1 BisAbs were able to mediate T cell cytotoxicity against the target, T2 cells pulsed with or without pIRS2, WT-IRS2 or control HLA-A*02:01-binding peptide EW, were incubated with human PBMCs used as effectors, in the presence or absence of the 6B1 BisAbs or control hIgG1 isotype (FIGS. 5A-H). While there was considerable variability in potency, all 6B1 BisAbs mAb mediated specific, effective killing against T2 cells pulsed with pIRS2 peptide, (FIG. 5B), but not T2 cells alone (FIG. 5A), WT-IRS2 (FIG. 5C) or pulsed with control peptide EW (FIG. 5D). Interestingly, the C2+1 form of 6B1 BisAb was not better than the native hIgG1. BisAbs 6B1 1+1 and H2+2 were consistently most potent. The ability of the BisAbs to mediate enhanced cytolytic activity against naturally presented epitopes on cancer cells was then tested. BisAbs 6B1 1+1 and H2+2 showed effective killing against all three cell lines MDA-MB-231, SKOV-3 and TPC-1 (that are pIRS2/HLA-A2+), but not against the A375 cell line, which is pIRS negative, WT-IRS2 and HLA-A2 positive (FIGS. 5E-5H). The order of potency of the BisAbs was generally similar to that observed against the pulsed cell line targets. The T cell-mediated cytotoxicity against target cells BV173, SKOV-3, MDA-MB-231, Jeko, NCEB1 and SET-2 by 6B1 1+1 was further confirmed (FIG. 8). These results demonstrated that by enhancing the effector function with T cells as effector cells, BisAbs in various formats were able to overcome the obstacle of low target density on tumor cells resulting in the cytotoxicity against the target. Interestingly, although all the 6B1 BisAs should be able to recruit T cells as effector cells, depending on the design and geometry of the constructs, T cell-mediated killing activity varied significantly.

The 6B1-mediated cytotoxicity against primary AML cells was assessed by using fresh frozen patient-derived samples (FIG. 5I). All the samples have more than 90% of CD33 positive cells, confirming their AML cell origin. An example of 6B1 binding was shown in FIG. 15C, in which 21% of CD33 positive AML cells were bound by the 6B1 mAb. Two HLA-A2 positive samples were variably killed by the mAb 6B1 (1+1) in the presence of activated T cells at an E:T ratio 6:1, and no cytotoxicity was observed against two samples that were HLA-A2 negative. These data demonstrated that this bispecific mAb is able to specifically kill the AML cells in an HLA-A2-restricted manner (FIG. 5I).

These results demonstrate that the compositions of the present technology are useful in methods for treating pIRS2-associated diseases (e.g., cancers) in a subject in need thereof.

### Example 7: Recognition of Phosphopeptides with Similar Amino Acid Compositions by 6B1

HLA-A2-binding phosphopeptides have been shown to possess unusual characteristics. A single phosphorylated serine residue (p-Ser) or phosphorylated threonine reside (p-Thr) are located at position 3-9, of which, 68% are found at position 4. In addition, 62% of the phosphopeptides have positively charged amino acids arginine or lysine at P1; in contrast, only 9-12% of nonphosphorylated HLA-A2 epitopes derived from either the Immune Epitope database or a set of naturally processed peptides extracted from B lymphoidblastoid cells have a positively charged amino acid at this position. This raised the question whether 6B1 could recognize other HLA-A2 bound phosphopeptides with an arginine in position 1 and a phosphoserine in position 4. 12 HLA-A2 bound phophopeptides were selected to pulse onto T2 cells. Of these peptides, nine have arginine at pos1 and p-Ser at position 4; 6B1 bound to 6 out of 9 peptides at different levels. No binding by 6B1 was observed in T2 cells pulsed with three peptides pKMD (KMDpSLDMQ; SEQ ID NO: 40), CCCK (KLIDIVpSSQKV; SEQ ID NO: 41) and b-catenin (YLDpSGIHSGA; SEQ ID NO: 42). None of these latter three peptides have arginine at pos1, even though pKMD and b-catenin shared the p-Ser at pos4. These results further demonstrated that pos1 R and pos4 p-Ser are likely both required residues for recognition by 6B1. However, other amino acids in the context of the peptides also play a role in the 6B1 recognition, as the large variation in binding demonstrates (FIG. 11).

These results demonstrate that the compositions of the present technology are useful in methods for treating pIRS2-associated diseases (e.g., cancers) in a subject in need thereof.

### Example 8: Modeling of Recognition of Phosphopeptides

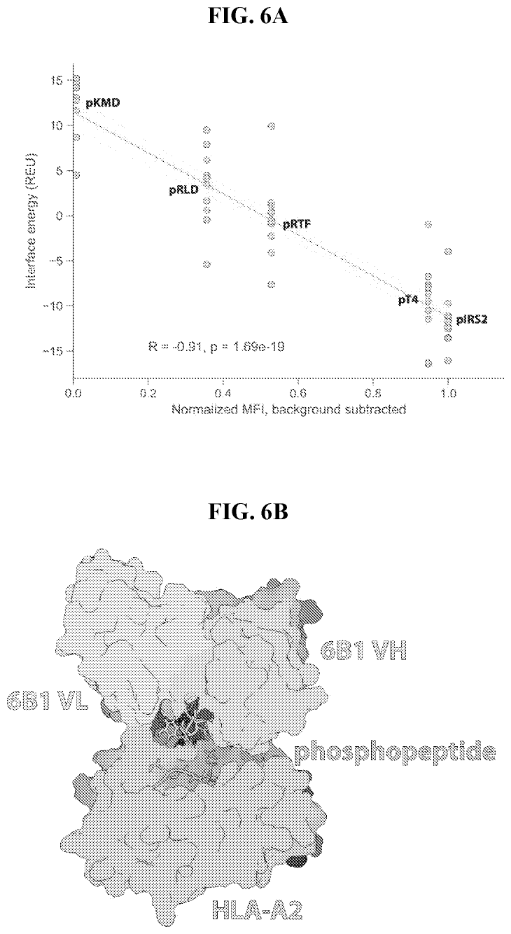

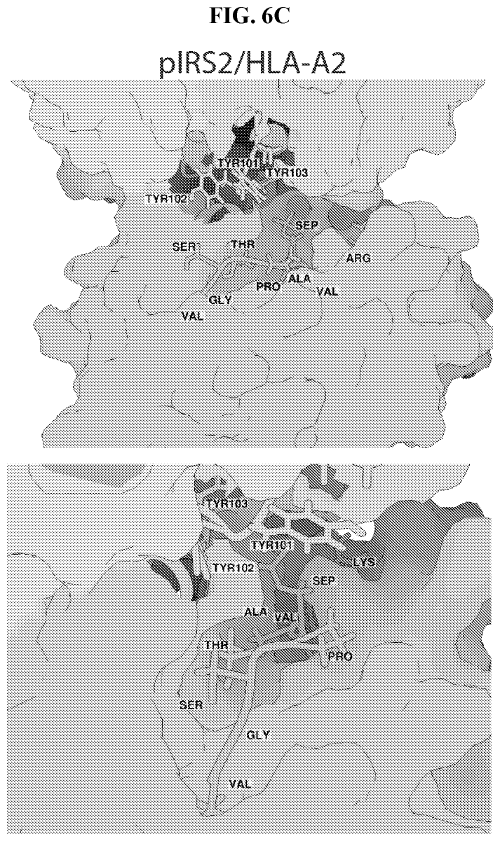

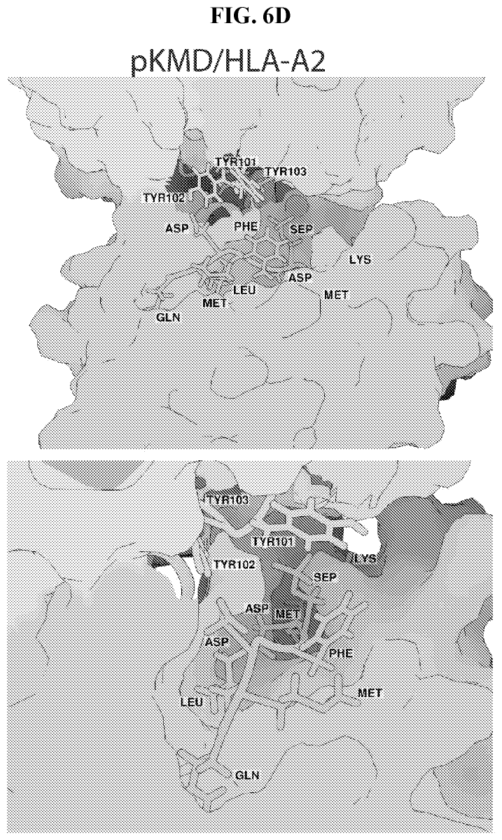

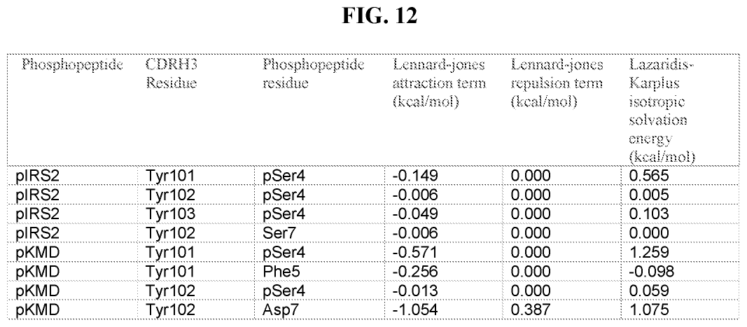

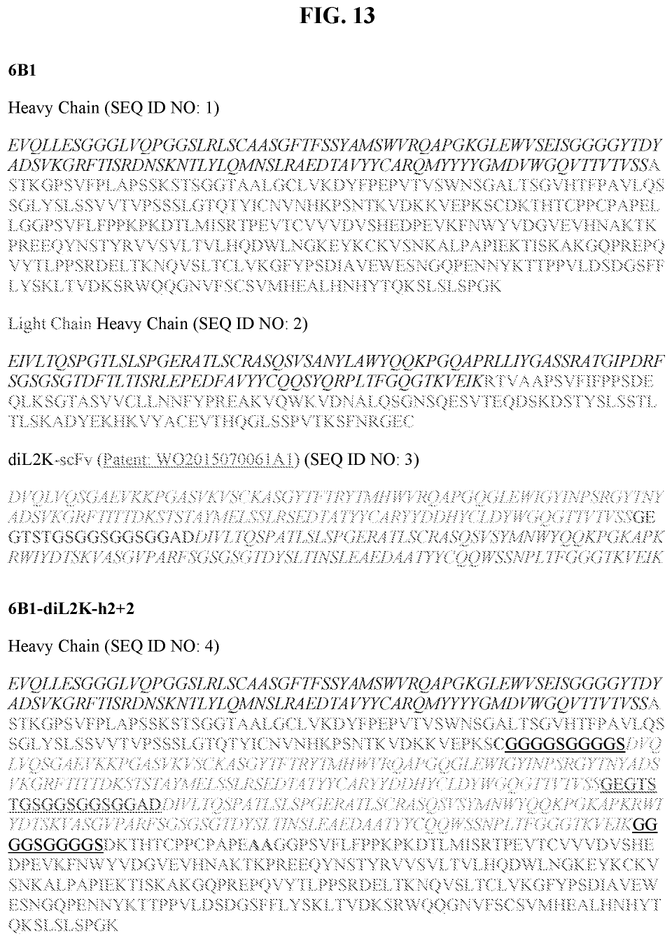

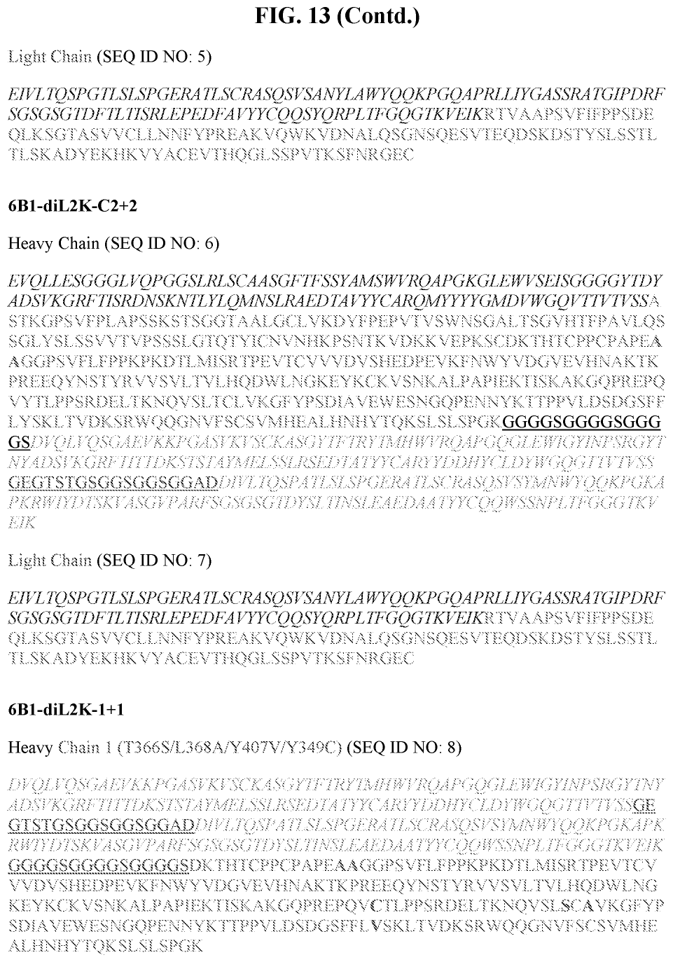

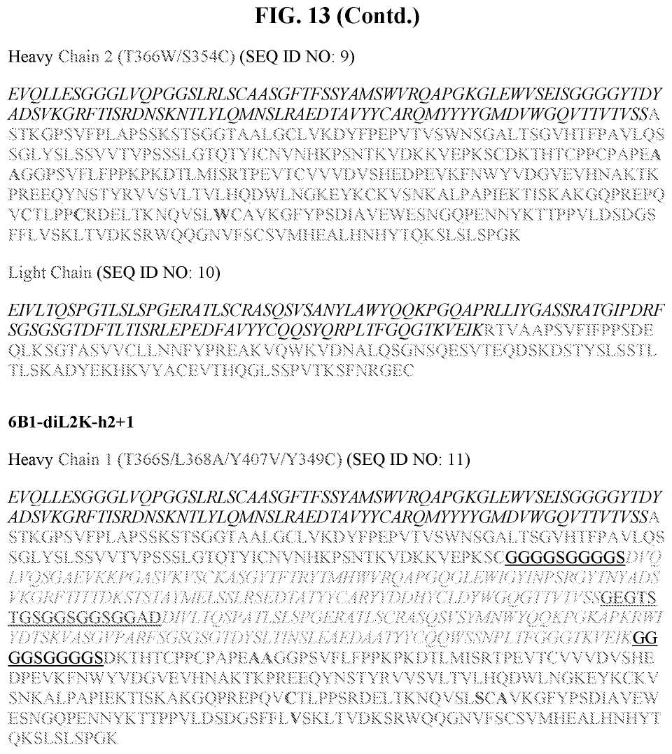

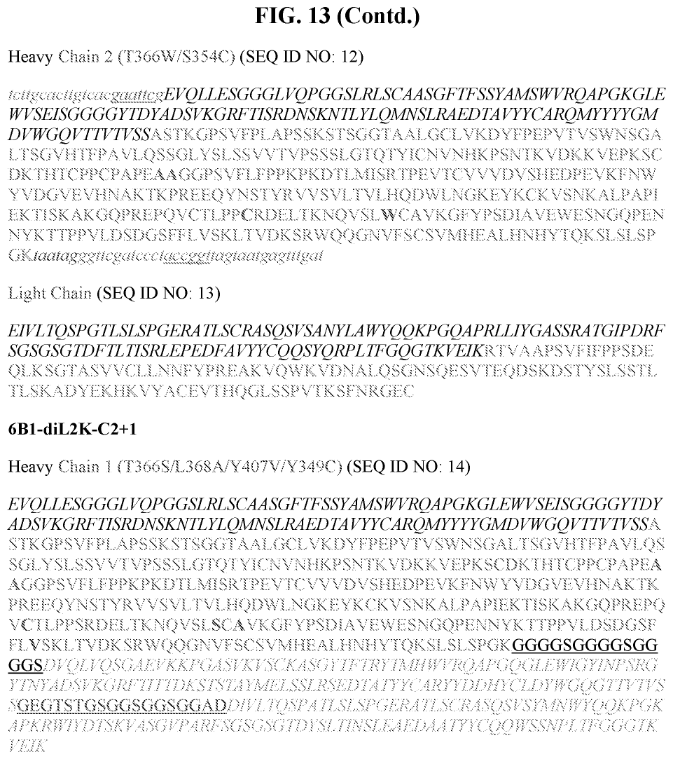

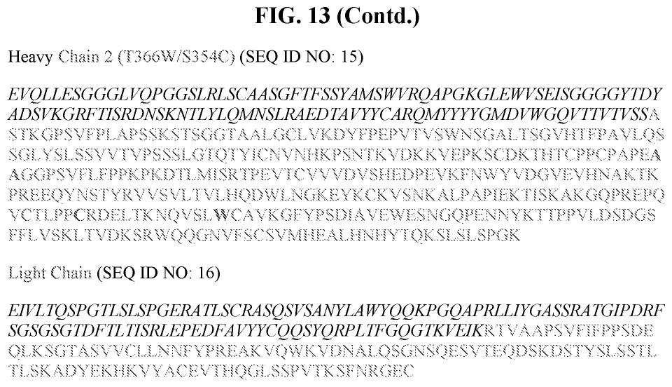

The variation in binding of 6B1 to different phosphopeptide/HLA-A2 complexes was investigated by computational modeling. Structural models of the 6B1 variable regions were generated using LYRA¹ and docked to HLA-A2 in complex with pIRS2, pT4, pKMD, and pRTF using the FlexPepDock web server to determine the energetic favorability of 6B1 binding to each phosphopeptide/HLA-A2 complex. The 10 lowest energy models for each complex were selected, and the energy of their respective binding interfaces were compared, yielding a clear correlation with in vitro binding of 6B1 to each phosphopeptide/HLA-A2 complex (FIG. 6A). To further determine the CDR residues contributing to 6B1's specificity, the inter-residue contacts between the CDRH3 of 6B1 and the phosphopeptide/HLA-A2 complex were examined. When 6B1 is bound to its cognate pIRS2/HLA-A2, Tyr101A of its CDRH3 experiences a favorable Lennard-Jones attraction energy of −0.149 kcal/mol and Lazaridis-Karplus isotropic solvation energy of 0.565 kcal/mol with pSer4 of pIRS2 (FIG. 6C). When bound to pKMD/HLA-A2, Tyr101A experiences an even stronger attractive potential of −0.571 kcal/mol for pSer4 (FIG. 6D); however, this is offset by a higher solvation energy of 1.259 kcal/mol. Other inter-residue contacts between phosphopeptide residues and Tyr101 and Tyr102 of CDRH3 are also likely to play a role in 6B1 binding, such as the unfavorable repulsion and solvation energies of Asp7 of pKMD with Tyr102 of CDRH3 (FIG. 12). Based off of this computational modeling and the binding data in vitro, these results show that 6B1 target binding is thermodynamically favorable when HLA-A2-bound phosphopeptides harbor a phosphate moiety at position 4 flanked by residues that enable favorable desolvation.

These results demonstrate that the compositions of the present technology are useful in methods for treating pIRS2-associated diseases (e.g., cancers) in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A composition that comprises an antibody moiety comprising: a heavy chain immunoglobulin variable domain (V_{H}) comprising a V_{H}-CDR1 sequence, a V_{H}-CDR2 sequence, and a V_{H}-CDR3 sequence of the V_{H} sequence of SEQ ID NO: 17, and a light chain immunoglobulin variable domain (V_{L}) comprising a V_{L}-CDR1 sequence, a V_{L}—CDR2 sequence, and a V_{L}-CDR3 sequence of the V_{L} sequence SEQ ID NO: 18.

2. The composition of claim 1, wherein the V_{H}-CDR1 sequence comprises the sequence of SEQ ID NO: 20, the V_{H}-CDR2 sequence comprises the sequence of SEQ ID NO: 21, the V_{H}-CDR3 sequence comprises the sequence of SEQ ID NO: 22, the V_{L}-CDR1 sequence comprises the sequence of SEQ ID NO: 23, the V_{L}-CDR2 sequence comprises the sequence of SEQ ID NO: 24, and the V_{L}-CDR3 sequence comprises the sequence of SEQ ID NO: 25.

3. The composition of claim 1, wherein (a) the V_{H} comprises an amino acid sequence having at least 90% identity to SEQ ID NO: 17, and/or (b) the V_{L} comprises an amino acid sequence having at least 90% identity to SEQ ID NO: 18; or
wherein (a) the V_{H} comprises an amino acid sequence of SEQ ID NO: 17 or a variant thereof having one or more conservative amino acid substitutions; and/or (b) the V_{L} comprises an amino acid sequence of SEQ ID NO: 18 or a variant thereof having one or more conservative amino acid substitutions.

4. (canceled)

5. The composition of claim 1, comprising
an amino acid sequence having at least 90% identity to a sequence selected from the group consisting of: SEQ ID NOs: 1-2, or 4-16; or
an amino acid sequence selected from the group consisting of: SEQ ID NOs: 1-2, or 4-16.

6. (canceled)

7. (canceled)

8. The composition of claim 1, further comprising a Fc domain of an isotype selected from the group consisting of IgG1, IgG2, IgG3, IgG4, IgA1, IgA2, IgM, IgD, and IgE; or wherein the antibody moiety is a full-length antibody, a Fab, a F(ab′)₂, a Fab′, a Fᵥ, or a single chain Fv (scFv).

9. (canceled)

10. The composition of claim 1, wherein the composition
is a chimeric antibody-T cell receptor (caTCR); or
comprises at least a fragment of a T cell receptor (TCR) chain, optionally wherein the fragment of the TCR chain comprises the transmembrane domain of the TCR chain and/or does not comprise any CDR sequence of the TCR chain; or
is a chimeric antigen receptor (CAR); or
is monospecific; or
is multi-specific or bispecific; or
comprises a tandem scFv, a diabody (Db), a single chain diabody (scDb), a dual-affinity retargeting (DART) antibody, a dual variable domain (DVD) antibody, a knob-into-hole (KiH) antibody, a dock and lock (DNL) antibody, a chemically cross-linked antibody, a heteromultimeric antibody, or a heteroconjugate antibody; or
comprises a tandem scFv with at least one peptide linker between two scFvs; or
comprises a second antibody moiety that specifically binds to a second antigen, optionally wherein the second antigen is an antigen on the surface of a T cell, a natural killer cell, a neutrophil, a monocyte, a macrophage, or a dendritic cell or wherein the second antigen is a disease-specific antigen that is not pIRS2/MHC; or
is a chimeric antibody, a humanized antibody, or a human antibody; or
is a fully human antibody or a monoclonal antibody; or
is an immunoglobulin-related composition, an immunoglobulin polypeptide, or an immunoglobulin-like polypeptide; or
specifically binds to a pIRS2 peptide/HLA-A*02 complex, optionally wherein said pIRS2 peptide comprises the amino acid sequence RVA[pS]PTSGVK (SEQ ID NO: 19) and/or wherein said HLA-A*02 is HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:13, HLA-A*02:16, HLA-A*02:18, HLA-A*02:19, HLA-A*02:28, or HLA-A*02:50.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A recombinant nucleic acid or a set of recombinant nucleic acids encoding the composition of claim 1, with all components of the composition encoded by one nucleic acid or by the set of nucleic acids.

31. A vector comprising the recombinant nucleic acid of claim 30.

32. A set of vectors comprising the set of recombinant nucleic acids of claim 30.

33. A cell comprising the recombinant nucleic acid or the set of recombinant nucleic acids of claim 30.

34. A cell that displays on its surface or secretes the composition of claim 1, optionally wherein the cell is a T cell, a NK cell, a B cell, or a monocyte/macrophage.

35. (canceled)

36. A pharmaceutical composition comprising the composition of claim 1 and a pharmaceutically-acceptable carrier.

37. The composition of claim 1, wherein the composition is conjugated to an agent selected from the group consisting of detectable label, isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

38. A method for treating a pIRS2-associated disease in a subject in need thereof, comprising administering to the subject an effective amount of the composition of claim 1.

39. A method for treating a pIRS2-associated disease in a subject in need thereof, comprising administering to the subject an effective amount of the recombinant nucleic acid or the set of recombinant nucleic acids of claim 30.

40. A method for treating a pIRS2-associated disease in a subject in need thereof, comprising administering to the subject an effective amount of the cell of claim 34.

41. A method for treating a pIRS2-associated disease in a subject in need thereof, comprising administering to the subject an effective amount of the pharmaceutical composition of claim 36.

42. The method of claim 38, further comprising administering to the subject an effective amount of interferon-γ or
separately, sequentially or simultaneously administering to the subject an additional therapeutic agent, optionally wherein the additional therapeutic agent is one or more of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, immune checkpoint inhibitors, monoclonal antibodies that specifically target tumor antigens, T-cell therapy, immune activating agents, oncolytic virus therapy and cancer vaccines.

43. The method of claim 38, wherein the pIRS2-associated disease is a cancer, optionally wherein the cancer is acute lymphoblastic leukemia (ALL), acute myeloid/myelogenous leukemia (AML), Diffuse large B-cell lymphoma (DLBCL), peripheral T-cell lymphoma (PTCL), Burkitt's lymphoma, T cell lymphoma, B cell lymphoma, multiple myeloma, ovarian cancer, breast cancer, cervical cancer, prostate cancer, melanoma, mesothelioma, pancreatic cancer, thyroid cancer, liver cancer, hepatocellular carcinoma, or a cancer presenting the peptide of RVA[pS]PTSGVK (SEQ ID NO: 19) in complex with HLA-A*02.

44. (canceled)

45. (canceled)

46. (canceled)

47. (canceled)

48. A method for detecting pIRS2 expression levels in a biological sample comprising (a) contacting the biological sample with the composition of claim 1; and (b) detecting binding to a pIRS2 peptide-HLA-A*02 complex in the biological sample, optionally wherein the pIRS2 peptide comprises the amino acid sequence RVA[pS]PTSGVK (SEQ ID NO: 19).

49. (canceled)

50. (canceled)

51. (canceled)

52. (canceled)
